(19) United States
(12) Patent Application Publication
(10) Pub. No.: US 2017/0304368 A1
(43) Pub. Date: Oct. 26, 2017

(54) POLARIZATION OF MACROPHAGES TO A HEALING PHENOTYPE BY CARDIOSPHERE-DERIVED CELLS AND BY THE EXOSOMES SECRETED BY SUCH CELLS

(71) Applicant: Cedars-Sinai Medical Center, Los Angeles, CA (US)

(72) Inventors: Eduardo Marban, Santa Monica, CA (US); Geoffrey DeCouto, Los Angeles, CA (US); Eleni Tseliou, Los Angeles, CA (US)

(73) Assignee: Cedars-Sinai Medical Center, Los Angeles, CA (US)

(21) Appl. No.: 15/517,140

(22) PCT Filed: Oct. 6, 2015
(86) PCT No.: PCT/US15/54301

§ 371 Date: Apr. 5, 2017

Related U.S. Application Data
(60) Provisional application No. 62/060,481, filed on Oct. 6, 2014.
(60) Provisional application No. 62/060,452, filed on Oct. 6, 2014.

(51) Int. Cl. A61K 35/34; A61K 9/00; C12N 5/077; C12N 15/113; A61K 31/7088

(52) U.S. Cl. CPC A61K 35/34; C12N 15/113; A61K 31/7088; C12N 5/0656; A61K 9/0019; C12N 2310/141; C12N 2320/32; C12N 2502/1329

(57) ABSTRACT

Described herein are compositions and techniques related to generation and therapeutic application of stem cell-derived exosomes. The Inventors have discovered cardiosphere-derived cells (CDCs) and their secreted exosomes mediate such inflammatory processes, by, for example, shifting macrophages away from a proinflammatory M1 phenotype toward M2 healing phenotype. This suggests compositions and techniques for use in both long-term reversal of heart and vascular disease pathology, and protection against such disease progression via modulation of inflammation and immune responses.

### STATEMENT REGARDING FEDERALLY-SPONSORED RESEARCH

This invention was made with government support under R01 HL083109 awarded by the National Institutes of Health. The government has certain rights in the invention.

### FIELD OF THE INVENTION

This invention relates to the use of cells and their extracts, specifically cellular exosomes, for therapeutic use, including treatment of heart disease.

### BACKGROUND

In injury models such as myocardial infarct, administration of cardiosphere-derived cells (CDCs) appears to promote newly regenerated myocardium and vasculature of endogenous origin. However, only long-term post-myocardial infarct endpoints have been studied. The cardioprotective effects of these cells in isolation is not well-understood, including possible modulation of inflammatory processes, such as macrophage response to injury. Further, there is growing evidence that the positive therapeutic benefits of CDCs occurs through indirect mechanisms. It is likely that such mechanism involve secretion of positive factors encapsulated within cellular exosomes produced by CDCs, the lipid bilayer nanovesicles secreted by cells when multivesicular endosomes fuse with the plasma membrane. Deciphering the role of secreted exosomes in potentiating CDC activity is a compelling area of interest, and in particular, the existence of a nexus between CDC-derived exosomes, cardioprotection and immune response remains unknown.

Understanding these processes governing CDC-initiated cardioprotection and regeneration may open new avenues for therapeutic approaches. For example, it appears that CDC-derived exosome therapy would provide broad benefits to heart disease broadly, based on several factors including superior dosage regimes (e.g., concentration, persistence in local tissue milieu, repeat dosages), and reduced or obviated safety concerns as non-viable entities. Establishing a role for CDC-derived exosomes in cardioprotection, for example, may find significant use as adjunctive therapy, given the relative ease of use when administering such compositions. This includes, for example, administration to post-infarct to limit in size. Longer-term disease repair and regeneration would also dramatically benefit those conditions currently lack any treatment modality. This includes preventing or reversing adverse arteriolar damage observed in pulmonary arterial hypertension (PAH), wherein cell-based therapies essentially cannot access or repair microvascular architecture. Similarly, patients suffering from Duchenne muscular dystrophy heart failure are not candidates for mechanical, tissue or organ transplant, and any treatment approach accessible to these subjects may deliver dramatic improvements.

Described herein are compositions and techniques related to generation and therapeutic application of CDC-derived exosomes. The Inventors discovered that CDCs are capable of attenuating cardiomyocyte apoptosis and protecting ventricular myocytes from oxidative stress by modifying the myocardial leukocyte population after ischemic injury. In reducing the number of CD68+ macrophages (Mφ) and polarize Mφ towards a distinctive (non-M1 or -M2) cardioprotective phenotype, it appears the release of secretory factors via exosomes allows delivery of a unique milieu of biological factors serving to mediate many of the therapeutic effects of stem cells such as CDCs. Importantly, the Inventors have established that exosomes can alter Mφ status towards cardioprotection, thereby implicating a direct role for exosomes in inflammatory processes following injury.

### SUMMARY OF THE INVENTION

Described herein is a method of modulating inflammation, including selecting a subject afflicted with an inflammatory related disease and/or condition; and administering a composition including a plurality of exosomes to the subject, wherein administration of the composition modulates inflammation in the subject by polarizing an endogenous population of macrophages in the subject. In other embodiments, the inflammatory related disease and/or condition is acute. In other embodiments, the inflammatory related disease and/or condition is chronic. In other embodiments, the inflammatory related disease and/or condition is a heart related disease and/or condition. In other embodiments, the heart related disease and/or condition is myocardial infarct. In other embodiments, the heart related disease and/or condition is atherosclerosis and/or heart failure. In other embodiments, polarizing an endogenous population of macrophages includes appearance of M_{CDC} macrophage phenotype, decreased M1 macrophage phenotype and/or increased M2 macrophage phenotype. In other embodiments, the M_{CDC} macrophage phenotype includes expression of one or more of interleukin-10 (Il10) and interleukin-4ra (Il4ra), M1 macrophage phenotype includes expression of one or more of nitric oxidate synthase (Nos2), tumor necrosis factor (Tnf), interleukin-1 (Il1), and interleukin6 (Il6), and M2 macrophage phenotype includes expression of one or more of arginase 1 (Arg1), interleukin-10 (Il10), and peroxisome proliferator-activated receptor gamma (Pparg). In other embodiments, decreased M1 macrophage phenotype and/or increased M2 macrophage phenotype includes an increase in Arg1/Nos2 ratio in a population of macrophages. In other embodiments, decreased M1 macrophage phenotype and/or increased M2 macrophage phenotype includes a decrease in Ly6C expression in a population of macrophages. In other embodiments, the macrophages are from cardiac tissue, peritoneum, spleen and/or bone marrow. In other embodiments, administering a composition includes 1×10⁸ or more exosomes in a single dose. In other embodiments, a single dose is administered multiple times to the subject. In other embodiments, administering a composition consists of one or more of: intra-arterial infusion, intravenous infusion, percutaneous injection, and injection directly into heart tissue.

Further described herein is a method of conferring cardioprotection, including selecting a subject afflicted with myocardial infarct (MI), ischemia/reperfusion (IR), or both and administering a composition including a plurality of exosomes to the subject, wherein the plurality of the exosomes are isolated from cardiosphere-derived cells (CDCs) grown in serum-free media, include one or more exosomes with a diameter of about 90 nm to about 200 nm and are CD81+, CD63+, or both, and further wherein administration of the composition confers cardioprotection by polarizing an endogenous population of macrophages in the subject. In other embodiments, the macrophages are from cardiac tissue, peritoneum, spleen and/or bone marrow. In other embodiments, administering a composition includes 1×10⁸ or more exosomes in a single dose. In other embodiments, a single dose is administered multiple times to the subject. In other embodiments, administering a composition consists of one or more of: intra-arterial infusion, intravenous infusion, percutaneous injection, and injection directly into heart tissue. In other embodiments, administering a composition including a plurality of exosomes to the subject is adjunctive to standard therapy. In other embodiments, administering a composition is less than 1 hour after reperfusion. In other embodiments, conferring cardioprotection reduces infarct size.

Further described herein is a method, including providing a population of cells including stem cells, progenitors, and/or precursor cells, and isolating a plurality of exosomes from the population of cells, wherein the plurality of exosomes include one or more exosomes with a diameter of about 90 nm to 200 nm, are CD81+, CD63+, or both, and are about 2-5 kDa. In other embodiments, the stem cells, progenitors, and/or precursor cells include cardiosphere-derived cells (CDCs) grown in serum-free media, and are confluent when isolating the plurality of exosomes. In other embodiments, the plurality of exosomes include one or more exosomes including one or more microRNAs selected from the group consisting of: miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In other embodiments, isolating the plurality of exosomes includes precipitation, centrifugation, filtration, immuno-separation, and/or flow fractionation.

Also described herein is a composition produced by the method including providing a population of cells including stem cells, progenitors, and/or precursor cells, and isolating a plurality of exosomes from the population of cells, wherein the plurality of exosomes include one or more exosomes with a diameter of about 90 nm to 200 nm, are CD81+, CD63+, or both, and are about 2-5 kDa. Further described herein is a the stem cells, progenitors, and/or precursor cells include cardiosphere-derived cells (CDCs) grown in serum-free media, and are confluent when isolating the plurality of exosomes.

Further described herein is an in vitro method of altering a cell, including providing a plurality of exosomes and adding to a starting cell type, the plurality of exosomes, wherein adhesion between one or more exosomes in the plurality of exosomes and the starting cell type is capable of altering one or more properties of the starting cell type, and generating a converted cell type. In other embodiments, the plurality of exosomes are derived from stem cells, progenitors, and/or precursor cells. In other embodiments, the stem cells, progenitors, and/or precursor cells include cardiosphere-derived cells (CDCs). In other embodiments, the stem cells, progenitors, and/or precursor cells include endothelial precursor cells (EPCs) and/or mesenchymal stem cells (MSCs). In other embodiments, the starting cell type is a fibroblast.

Also described herein is a quantity of converted cells made by the in vitro method of altering a cell, including providing a plurality of exosomes and adding to a starting cell type, the plurality of exosomes, wherein adhesion between one or more exosomes in the plurality of exosomes and the starting cell type is capable of altering one or more properties of the starting cell type, and generating a converted cell type.

Further described herein is an in vivo method of altering a cell, including selecting a subject and administering a composition including a plurality of exosomes to the subject, wherein adhesion between one or more exosomes in the plurality of exosomes and a starting cell type is capable of altering one or more properties of the starting cell type, thereby generating a converted cell type in vivo. In other embodiments, the plurality of exosomes are derived from stem cells, progenitors, and/or precursor cells. In other embodiments, the stem cells, progenitors, and/or precursor cells include cardiosphere-derived cells (CDCs). In other embodiments, the stem cells, progenitors, and/or precursor cells include endothelial precursor cells (EPCs) and/or mesenchymal stem cells (MSCs). In other embodiments, the starting cell type is a fibroblast.

### BRIEF DESCRIPTION OF FIGURES

FIG. 1. Differential Expression of microRNAs in Cardiosphere-Derived Cell Exosomes. (A) MicroRNA analysis of CDC-derived exosomes demonstrate the differential cargo contents of exosomes based on parental cellular origin. Fold changes of microRNA abundance in CDC exosomes compared to normal human dermal fibroblasts (NHDF) exosomes (n=4 independent experiments). Total RNA (including microRNAs) was isolated from CDC exosomes and NHDF exosomes. qRT-PCR was performed on an microRNA array. (B) Venn diagram showing the variable microRNA profile between CDC and NHDF exosomes. Font size reflects the magnitude of differential expression of each microRNA.

FIG. 2. Isolation of Exosomes from CDCs. (A) Graphical representation of exosome isolation and purification for exosomes. (B) Cell viability (calcein) and cell death (Ethidium homodimer-1) assay performed on CDCs over the 15 day serum-free conditioning period. (C) Representative images of CDCs before and after serum-free conditioning.

FIG. 3. Heat Map or microRNA PCR Array Identifies Mir-146a as a Highly Differentially Expressed microRNA. Heat map showing fold regulation differential abundance data for transcripts between CDC exosomes and NHDF exosomes overlaid onto the PCR Array plate layout.

FIG. 4. CDCs confer cardioprotection to the ischemic myocardium within 20 minutes of reperfusion. (A) Schematic of infusion protocol. Rats underwent 45 minutes of ischemia followed by either 20 minutes or 120 minutes (delayed injection) of reperfusion prior to infusion of CDCs (5×10⁵/100 μL) or PBS control (100 μL) into the LV cavity with an aortic cross-clamp. Animals were assessed 48 hours later. (B) Ejection fraction, as measured by echocardiography, is significantly preserved in CDC-treated animals at 48 hours with 20 minutes, but not 120 minutes, delay of infusion. (C) Representative TTC-stained hearts from animals at 48 hours following IR injury. (D) Quantitative measurements of TTC-stained hearts, depicted as percent infarct mass (%), infarct mass (g), LV mass (g), and LV viable mass (g). Graphs depict mean±SEM. Statistical significance was determined using 1-way ANOVA followed by Bonferroni's multiple comparisons test. *p<0.05. (E) Representative images of gentian violet- and TTC-stained hearts isolated from rats 48 hours following treatment with PBS or CDCs (5×105) and (F) quantitative analysis of the AAR (black line), infarct area (gray line), and LV area of TTC-stained hearts (n=6-7 rats per group). (G) Linear regression of percentage of infarct mass vs. AAR shows no significant interaction when analyzed for homogeneity of regressions (F=0.23, P=0.64) but a difference between adjusted means of infarct mass (F=84.5, P<0.001) by analysis of covariance. Graphs depict mean±SEM. Statistical significance was determined using 1-way ANOVA followed by Bonferroni's multiple comparisons test. *P<0.05.

FIG. 5. The acute cardioprotective effect of CDCs is sustained until 2 weeks following IR. (A) Schematic of infusion protocol. Rats underwent 45 minutes of ischemia followed by 20 minutes of reperfusion before infusion of CDCs or PBS control. Animals were followed for 2 weeks for long-term analyses. (B) Representative echocardiography long-axis traces of the LV cavity during diastole and systole from PBS- and CDC-treated animals. (C) Masson's trichrome staining of infarcted hearts from PBS- and CDC-treated animals. (D) Pooled data from echocardiographic assessments prior to (pre-ischemia) and following (2 weeks) IR injury. Ejection fraction (%), end-diastolic volume (μL), and end systolic volumes (μL) were preserved in CDC-treated animals. (E) Pooled data from Masson's trichrome-stained hearts in (C) reveal less infarct thinning in CDC-treated animals. (F) Immunohistochemical staining of cardiomyocytes in the contralateral infarct zone. Cell size was determined from cardiomyocytes (α-Actinin+WGA) with centrally-localized nuclei (DAPI). (G) Pooled data from analyses in (F) depicting a reduction in cardiomyocyte size in CDC-treated animals. Graphs depict mean±SEM. Statistical significance was determine using Student's t-test and 2-way ANOVA followed by Bonferroni's multiple comparisons test. *p<0.05.

FIG. 6. Infusion of CDCs post-IR reduces cardiomyocyte death and alters the tissue proinflammatory cytokine expression. (A) Schematic of infusion and tissue harvest protocol. As previously described, animals underwent 45 minutes of ischemia, followed by 20 minutes of reperfusion prior to PBS or CDC delivery. Animals were sacrificed for analyses after 2, 6, or 48 hours of IR injury. (B) Representative protein immunoblots of cleaved caspase 3, caspase 3, RIP, and GAPDH from the normal (N), border (B), and infarct (I) zones of hearts treated with PBS and CDCs. (C) Pooled data from immunoblots in (B) reveal a reduction in caspase 3 activation and RIP expression levels in the infarct region of CDC-treated hearts. (D) Representative images of TUNEL-stained heart tissue from the infarct zones of PBS- and CDC treated hearts. (E) Quantitative assessment cardiomyocytes in (D) reveal reduced TUNEL positivity in CDC-treated hearts at all time points. (F) Protein cytokine expression of MMP8 and CXCL7 is elevated in the infarct zone of hearts treated with CDCs. Graphs depict mean±SEM. Statistical significance was determined using either 1-way or 2-way ANOVA followed by Bonferroni's multiple comparisons test. *p<0.05.

FIG. 7. CDC-treated animals have a reduced CD68⁺ MΦ population 48 hours post-IR. (A) Gating strategy for leukocyte identification within the infarcted myocardium prior to subset analysis. CD45⁺ were first identified (FSC-A/CD45⁺) and then dead cells excluded (DAPI⁻). (B) Pooled flow cytometry data from infarcted rat tissue reveal a reduced CD68⁺ population in CDC- vs. PBS-treated hearts. (C) Immunohistochemical staining of hearts within the infarct zone from CDC- and PBS-treated animals at 2, 6, and 48 hours post-IR. (D) Pooled data of CD68⁺ cells within the infarct tissue (C) at 2, 6, and 48 hours post-IR. Graphs depict mean±SEM. Statistical significance was determined using Student's t-test and 2-way ANOVA followed by Bonferroni's multiple comparisons test. *p<0.05.

FIG. 8. Systemic depletion of endogenous MΦ reduces the efficacy of CDC therapy. (A) Schematic depicting the Mφ depletion protocol using clodronate (Cl₂MDP: dichloromethylene diphosphonate) liposomes. Animals were treated with an intravenous infusion of Cl₂MDP 1 day prior to, and one day following, IR injury and then assessed 48 hours following IR injury. (B) Representative TTC-stained heart from Cl₂MDP and PBS-treated animals 48 hours post-IR. Clodronate treatment led to trends towards an increase in infarct mass (C) and reduction in cardiac ejection fraction (D) in both PBS and CDC-treated animals relative to their untreated controls. Graphs depict mean±SEM. Statistical significance was determined using Student's t test and 1-way ANOVA followed by Bonferroni's multiple comparisons test. *p<0.05.

FIG. 9. Cardiac MΦ (cMΦ) isolated from CDC-treated animals have a distinct cytokine profile. (A) Representative images of CD68⁺ Mφ cells isolated from cardiac tissue of PBS and CDC-treated animals 48 hours following MI. (B) Pooled data from CD68⁺ staining of cMφ isolated in (A). Immunohistochemistry reveals a purity level of >85% CD68 positivity following cMφ isolation. (C) Gene expression profile from cMφ isolated from infarcted hearts after 48 hours. CDC-treated hearts have cMφ with reduced M₁ (Tnf, Nos2, Il1a, and Il1b), but no change in M₂ (Arg1, Tgfb1, and Il10), Mφ gene expression. Graphs depict mean±SEM. Statistical significance was determined using 2-way ANOVA followed by Bonferroni's multiple comparisons test. *p<0.05.

FIG. 10. Polarization of BM-derived MΦ toward M₁, M₂, or M_{CDC} in vitro confers distinct cytokine gene expression and surface marker expression. (A) Representative phase contrast images of Mφ polarized toward M₁ (IFNγ/LPS), M₂ (IL-4/IL-13), or M_{CDC} (transwell) phenotypes. (B) Gene expression profiles of Mφ polarized toward M₁, M₂, and M_{CDC}. These data reveal classical upregulation of markers in M₁ (Nos2) and M₂ (Arg1, Pparg, NJkb1, Tgfb1) Mφ, but with distinct gene expression in M_{CDC} (Il10) Mφ. (C) Protein expression of markers delineating M₁ and M₂ Mφ reveal a distinct expression pattern in M_{CDC} Mφ, (D & E) Surface markers examined by flow cytometry depict reduced CD68, CD80, and CD86 expression on M_{CDC} Mφ. Graphs depict mean±SEM. Statistical significance was determined using 1-way ANOVA followed by Tukey's multiple comparisons test. *p<0.05. (F) Protein expression of markers delineating M1 and M2 macrophages vs. MCDC macrophages (n=3 per group). Graphs depict mean±SEM. Statistical significance was determined using 1-way ANOVA followed by Tukey's multiple comparisons test. *P<0.05. group). (G and H) Representative flow cytometry histograms and pooled quantitative analysis of FITC fluorescent bead uptake among macrophage populations (n=3 per group). Graphs depict mean±SEM. Statistical significance was determined using 1-way ANOVA followed by Tukey's multiple comparisons test. *P<0.05.

FIG. 11. Co-culture of M_{CDC} MΦ with oxidatively-stressed NRVM preserves cardiomyocyte viability in vitro. (A) Schematic of in vitro protocol. NRVMs are stressed with 50 μM H₂O₂ for 15 minutes, serum-free media is replaced for 20 minutes (to simulate reperfusion), and then DiO-labeled M₁, M₂, or M_{CDC} Mφ are introduced to the NRVMs. After 6 hours, cells are collected for analyses. (B) Representative images of TUNEL-stained (red) cocultures of M₁, M₂, or M_{CDC} (green) with NRVMs (white). Pooled quantitative analyses of TUNEL⁺ cardiomyocytes (CM) (C) and viable nucleated CM (D) from M₁, M₂, and M_{CDC} cocultures. (E) Immunoblot of co-cultured cells (M₁, M₂, or M_{CDC} with H₂O₂-treated NRVMs) and NRVM positive and negative controls (with, and without, H₂O₂ respectively) after 6 hours of culture. (F) Quantitative analysis of immunoblots in (E). Graphs depict mean±SEM. Statistical significance was determined using 1-way ANOVA followed by Tukey's multiple comparisons test. *p<0.05. (G) Pooled data demonstrating increased macrophage numbers in M1 cocultures and increased TUNEL+macrophages in M2 cocultures. Graphs depict mean±SEM. Statistical significance was determined using 1-way ANOVA followed by Tukey's multiple comparisons test. *P<0.05.

FIG. 12. Adoptive transfer of M_{CDC} MΦ reduce infarct size when administered 20 minutes following reperfusion. (A) Schematic of infusion protocol. Rats underwent 45 minutes of ischemia followed by 20 minutes of reperfusion prior to administration of DiI-labeled M₁, M₂, or M_{CDC} Mφ. Analyses were performed 48 hours after IR injury. (B) Representative images of TTC stained hearts from M₁, M₂, or M_{CDC} Mφ treated hearts. (C) Pooled data of percent infarct mass and LV viable mass as assessed from TTC-stained hearts. (D) Representative image of the localization of DiI-labeled Mφ within the infarct border zone; no DiI-labeled Mφ were observed in the non-infarcted region. Graphs depict mean±SEM. Statistical significance was determined using 1-way ANOVA followed by Tukey's multiple comparisons test. *p<0.05.

FIG. 13. Leukocyte and cytokine profiling within the blood and heart 48 hours post-IR. (A) Pooled data from flow cytometric analysis of peripherally-circulating inflammatory cells. (B) Serum protein expression of MCP-1 and IL-4. (C) Pooled data from flow cytometry of leukocytes isolated from ischemic cardiac tissue. (D) Immunohistochemistry of CD68⁺ Mφ within the cardiac tissue of sham-operated animals. These animals were designated to receive either PBS or CDC therapy, but did not undergo IR. Graphs depict mean±SEM. Statistical significance was determined using Student's t-test. *p<0.05.

FIG. 14. In vivo depletion of Mφ. (A) Representative flow cytometry plots of the CD45⁺CD68⁺ population in the spleen and blood from Cl₂MDP- and PBS-treated animals. (B) Pooled flow cytometric data from spleen and blood depicting the percent reduction in CD68 Mφ in Cl₂MDP-treated animals. (C) Pooled data of LV mass from PBS−, CDC−, PBS+Cl₂MDP−, and CDC+Cl₂MDP-treated animals. Graphs depict mean±SEM. Statistical significance was determined using Student's t-test.

FIG. 15. CDC polarization of thioglycollate-elicited peritoneal Mφ (pMφ). (A) Schematic depicting the duration of transwell coculture prior to gene expression analysis of isolated pMφ. (B) Representative FACS plot and immunohistochemistry image depicting the purity of CD68⁺ pMφ following peritoneal lavage. (C) Pooled changes in gene expression of M1 and M2 markers observed in pMφ cocultured in transwell with CDC and PBS after 0, 6, or 24 hours. Graphs depict mean±SEM. Statistical significance was determined using 2-way ANOVA followed by Sidak's multiple comparisons test. *p<0.05.

FIG. 16. CDC primed pMφ reduce cardiomyocyte oxidative stress in vitro via paracrine signals. (A) Schematic depicting the priming of pMφ via transwell coculture with or without CDCs for 24 hours. NRVMs were then treated with H₂O₂ (50 μM), prior to transwell coculture with pMφ. After 6 hours, NRVMs were collected for protein and gene expression analyses. (B) Immunoblots depicting the reduction in stress (pJNK, pp65) and apoptosis (caspase 8, caspase 3) marker expression in CDC-primed Mφ. (C) Pooled changes in protein expression of immunoblots in (B). (D) Changes in cardiomyocyte stress-associated gene expression of CDC-primed versus PBS-primed pMφ (pooled n=3/group). Graphs depict mean±SEM. Statistical significance was determined using 1-way ANOVA followed by Tukey's multiple comparisons test. *p<0.05.

FIG. 17. Distinct gene and protein expression profiles for BM-derived M₁, M₂, and M_{CDC} Mφ. (A) Pooled data of Mφ gene markers. (B) Pooled data of protein immunoblots for Mφ markers. Graphs depict mean±SEM. Statistical significance was determined using 1 way ANOVA followed by Tukey's multiple comparisons test. *p<0.05.

FIG. 18. BM-derived M₁, M₂, and M_{CDC} Mφ have distinct protein marker expression patterns. (A) Representative FACS plot depicting changes in cell surface expression of Mφ markers. (B) Pooled immunoblot data depicting a reduction of CD11b in M₂, increase of CD45^{int} in M₁, and reduced cell size (FSC—forward scatter) in M_{CDC} Mφ. Graphs depict mean±SEM. Statistical significance was determined using 1-way ANOVA followed by Tukey's multiple comparisons test. *p<0.05.

FIG. 19. M₁, M₂, and M_{CDC} Mφ have distinct cytoprotective and proliferative capacities in vitro and in vivo. (A) Pooled data depicting an increase in viable cardiomyocytes (CM) following coculture with H₂O₂-treated NRVMs. (B) Pooled data demonstrating increased Mφ numbers in M₁ cocultures and increased TUNEL⁺ Mφ in M₂ cocultures. (C) Pooled data depicting a reduction of CD68 expression in M_{CDC}, relative to M₁ or M₂, cocultured with NRVMs 6 hours following H₂O₂-treatment. (D) Pooled data depicting a reduction in infarct mass, but no change in LV mass, in M_{CDC}-treated animals relative to M₁ or M₂ 48 hours following IR injury. Graphs depict mean±SEM. Statistical significance was determined using 1-way ANOVA followed by Tukey's multiple comparisons test. *p<0.05.

FIG. 20. CDC exosomes recapitulate the cardioprotective function of CDCs following IR injury. Percent infarct mass was examined in animals treated with human exosomes derived from six different donors 220 (220Ex), YKT (YKTEx), 155 (155Ex), ZHM (ZHMEx), ZKN (ZKNEx), and AABM (AABMEx) and were compared to vehicle control (PBS, phosphate buffered saline) or CDCs (0.5×106). CDC exosomes were isolated using ExoQuick (EQ) from a 10 mL equivalent volume. Exosomes were delivered following 45 minutes of ischemia and 20 minutes of reperfusion by LV cavity injection with an aortic cross-clamp over a period of 20 seconds. Hearts were isolated after 48 hours, sectioned to ˜1 mm thickness, weighed, then stained with TTC (2,3,5-Triphenyltetrazolium chloride). Infarct area and mass were determined using ImageJ software.

FIG. 21. CDC exosomes reduce the number of infiltrating CD68+ macrophage within the infarcted myocardium 48 hours following IR injury. The number of infiltrating CD68+ macrophage were examined by immunohistochemistry within the infarct myocardium of animals treated with four different human exosome donors 220 (220Ex), 155 (155Ex), YKT (YKTEx), and ZHM (ZHMEx), and were compared to vehicle control (PBS, phosphate buffered saline). At least 5 fields of view were examined for CD68 positivity per sample.

FIG. 22. CDC exosomes shift the macrophage gene expression profile toward a distinct MCDC phenotype. Exosomes from two different donors 155 (155Ex) and 220 (220Ex) were compared to exosomes derived from a human fibrosarcoma cell line HT-1080 (HTEx) and human dermal fibroblasts (dFbEx). CDC exosomes isolated using ExoQuick (EQ) or ultrafiltration by centrifugation (UFC) were compared. Rat bone marrow (BM) cells were isolated, then cultured with m-CSF for one week prior to addition of exosomes derived from an equivalent volume of conditioned media (1 mL or 3 mL fraction). BM cells were treated overnight (˜18 hrs) with exosomes and then harvested for qRT-PCR gene expression analyses. The y-axis depicts fold-change in gene expression to the internal housekeeping gene HPRT and untreated control BM cells.

FIG. 23. (A) Extracellular membrane vesicles (EMVs) were isolated from cardiospheres (CSps) on day 3 post-plating by adding Exoquick precipitation solution. (B) Size distribution was analyzed by nanoparticle tracking analysis and pooled data for particle number and size quantification revealed an average size of 175×12-nm diameter vesicles. (C) Tetraspanin-bound beads were used to characterize the human CSp-derived EMVs (hCSp-EMVs). Representative histograms revealed expression of CD63, CD81, and CD9. EMVs stained for tetraspanins (green line) were compared to appropriate controls (orange/blue lines). (D) Human dermal fibroblasts (hDFs) were incubated with fluorescent dyed hCSp-EMVs for 24 h followed by confocal imaging. (E) z-stack image of DFs 24 h post-hCSp-EMV incubation revealed particle internalization. (F) Representative confocal images of DFs incubated with different concentrations of hCSp-EMVs and evaluation of fluorescent intensity at different time points postincubation revealed cells with EMV signal and EMV intensity per cell at 6 h (G and H), 12 h (I and J), and 24 h (K and L) that were dose but not time dependent. Scale bar=250 mm; n=3-5 high-power (20×) images per group. DAPI=4′,6-diamidino-2-phenylindole; DF=dermal fibroblast; WGA=wheat germ agglutinin.

FIG. 24. Western blot of hDFs 24 h post-incubation with 2 different concentrations of hCSp-EMVs showed reduced psmad2/3 (A), psmad4 (B), and snai1 (C). Glyceraldehyde 3-phosphate dehydrogenase (GAPDH) was used as a control. The experiment was performed in triplicate. Flow cytometry was used for phenotypic characterization of the hCSp-EMV-primed fibroblasts 24 h post-incubation. Representative fluorescence activated cell sorting (FACS) plots (D) and pooled data (E) revealed reduced fibroblast specific protein 1 (FSP1) and discoidin domain receptor 2 (DDR2) expression while CD105 and CD90 were not affected (n=3 in each group). Confocal images of hCSp-EMV-primed and -unprimed fibroblasts (F) showed enhanced density of the smooth muscle actin (SMA) cells and lower FSP1 density after CSp-EMV incubation (G). Scale bar=250 mm. (H) Scheme of the collected supernatant after hCSp-EMV treated fibroblasts; (I) enzyme-linked immunosorbent assay (ELISA) on the collected supernatant revealed increased levels of SDF-1 and VEGF in the post-primed fibroblasts. n=5 per group. *p<0.05 versus unprimed fibroblasts. FACS=fluorescence activated cell sorting; ISO=isotype control; smad=small mothers against decapentaplegic homolog; other abbreviations as in FIG. 1.

FIG. 25. Representative FACS plots of neonatal rat ventricular myocytes (NRVMs) were treated with (A) hDF-EMVs, (B) conditioned media from hCSp-EMV-primed hDFs, and (C) hCSp-EMVs for 72 h and stained with Annexin V to evaluate apoptosis. The experiment was performed in triplicate (n=3 for each group). (D) Pooled data for the NRVM apoptosis revealed higher viability in the hCSp-EMV-primed fibroblast and hCSp-EMVs groups compared to the DF-EMVs. (Histogram color=group in bar graph.) (E-G) Representative higher power images of the matrigel tube formation assay from (E) hDF-EMVs, (F) conditioned media from hCSp-EMV primed hDFs, and (G) hCSp-EMVs and (H) pooled data for tube quantification. Similarly, enhanced tube formation was observed in the latter 2 groups compared to the hDF-EMVs only. *p<0.05 versus DF. Scale bar=50 mm. Abbreviations as in FIGS. 23 and 24.

FIG. 26. Micro-ribonucleic acid (miRNA) with statistically significant fold changes are seen in (A) hCSps versus hDFs and (B) hCSp-EMV_{┐} primed DFs versus unprimed hDFs. Additionally, fold ratios of miRNA profiles from EMVs derived either from hCSps or unprimed hDFs (C) and fold changes in the miRNA cargo of the EMVs secreted by hCSp-EMV_{┐} primed hDFs versus unprimed hDFs (D) are seen. All p<0.05. n=3 per group. Abbreviations as in FIG. 23.

FIG. 27. According to the study timeline (A), myocardial infarction was induced in Wistar Kyoto rats; 1 month later, the animals were allocated to injection of vehicle (PBS; orange bars; n=6), rDFs (yellow; n=8), rDFs primed with rCSp-EMVs (green; n=8), or rCSp-EMVs only (blue; n=8). Functional follow-up and histological analysis were performed 1 month post-injection. At 1 month post-injection, rCSp-EMVs and rCSp-EMV primed rDFs showed significant improvement in cardiac function via ejection fraction (B) as well as better-maintained left ventricular end-systolic diameter (LVESD) via M-mode short-axis images (C) compared to control groups. Scar mass was evaluated by serial Masson's trichrome_{┐} stained sections from the left ventricle (D), and was significantly reduced in the rCSp-EMVs and the rCSp-EMV primed rDF groups compared to either control group (E). Significant differences also were observed in infarct wall thickness (F). *p<0.05 versus DFs; **p<0.05 versus PBS. PBS=phosphate-buffered saline; rCSp=rat cardiosphere; rDFs=rat dermal fibroblasts; other abbreviations as in FIG. 1.

FIG. 28. (A) Representative immunostained images from the infarct, border, and remote zones are presented for evaluation of microvessel and capillary density. In the infarct (B), border (C), and remote (D) zones, pooled data revealed enhanced von Willebrand factor (vWf)_{┐} positive capillary density in the rCSp-EMVs and rCSp-EMV primed rDF groups compared to both controls (left panels) and changes regarding SMA-positive vessels (right panels). n=5 in each of the groups. Scale bar=250 mm. *p<0.05 versus DFs; **p<0.05 versus PBS. Abbreviations as in FIGS. 1, 2, and 5.

FIG. 29. (A) Representative immunostained images from the border and remote zones were used for evaluation of cardiomyocyte diameter. Pooled data revealed no difference between the groups analyzed in the border (B) and remote (C) zones. n=5 in each of the groups. Scale bar=100 mm. ASA=a-sarcomeric actin; other abbreviations as in FIGS. 1 and 5.

FIG. 30. Cardiosphere-isolated exosomes were used to prime inert fibroblasts. Post-priming analysis of fibroblast bioactivity revealed amplification of their therapeutic properties including cardiomyogenic, angiogenic, antifibrotic, and regenerative effects.

FIG. 31. Here the Inventors show data in mice that splenic mononuclear cells (which include macrophages) are uniquely polarized following treatment with human CDC exosomes (CDCexo). To do so, the Inventors pretreated mice with an intraperitoneal injection of lipopolysaccharide (LPS), an acute inflammatory stimulus, then infused CDCexo, or human dermal fibroblasts (hdFbexo) into the carotid artery. Eighteen hours later, mice were sacrificed and spleens collected. Spleens were digested to obtain a mixed cellular suspension. Mononuclear cells were isolated by density gradient centrifugation and plating onto cell culture dishes. Following attachment, cells were collected for RNA isolation and cDNA synthesis. Quantitative RT-PCR was then performed to assess the gene expression levels of Il10 and Vegfa, both of which were found upregulated in CDCexo-treated, but not Fbexo-treated, animals.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul. 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

There is growing evidence that the positive therapeutic benefits of stem cells, such as cardiosphere-derived cells (CDCs) occurs through indirect mechanisms. In injury models such as myocardial infarct, administration of CDCs appears to promote newly regenerated myocardium and vasculature of endogenous origin. Perhaps due to the fact that CDCs are rich biological factories that secrete many growth factors and cytokines, the beneficial therapeutic effects of CDCs manage to persist long after the injected cells have been cleared. Of critical interest is understanding whether these positive factors may exist in cellular exosomes produced by CDCs, the lipid bilayer nanovesicles secreted by cells when multivesicular endosomes fuse with the plasma membrane. Confirming a role for secreted exosomes in these processes has yet to be considered, and understanding these processes governing CDC-initiated regeneration may open new avenues for therapeutic approaches. For example, it appears that CDC-derived exosome therapy would provide broad benefits to heart disease broadly, based on several factors including superior dosage regimes (e.g., concentration, persistence in local tissue milieu, repeat dosages), and reduced or obviated safety concerns as non-viable entities. Particular advantages may be dramatic for those conditions that currently lack any treatment modality. This includes preventing or reversing adverse arteriolar damage observed in pulmonary arterial hypertension (PAH), wherein cell-based therapies essentially cannot access or repair microvascular architecture. Similarly, patients suffering from Duchenne muscular dystrophy heart failure are not candidates for mechanical, tissue or organ transplant, and any treatment approach accessible to these subjects may deliver dramatic improvements.

Described herein are compositions and techniques related to generation and therapeutic application of CDC-derived exosomes. These biological molecules contain a unique milieu of biological factors based on their parental cell type of origin. This “cargo content”, including antigenic protein makers allows for isolating and segregating exosome populations of interest, including those enriched for microRNAs that serve to mediate many of the therapeutic effects of stem cells such as CDCs. Exosomes and their constituent microRNAs can favorably modulate apoptosis, inflammation and promote repair of vessel structures, leading to functional recovery and increased tissue viability. Thus, CDC-derived exosomes represent a novel “cell-free” therapeutic candidate for tissue repair. Stem cell-derived exosome therapy can address pathology of diseases in a way that conventional drug therapy has failed to date. Importantly, the Inventors have established that exosomes possess significant potency in modulating regeneration and repair mechanisms, as capable of transferring the salutary benefits to cells that are otherwise therapeutically inert.

Exosomes, secreted lipid vesicles containing a rich milieu of biological factors, provide powerful paracrine signals by which stem cells effectuate their biological effects to neighboring cells, including diseased or injured cells. Through the encapsulation and transfer of protein, bio-active lipid and nucleic acid “cargo”, there is increasing recognition that these natural delivery devices are capable of inducing significant phenotypic and functional changes in recipient cells that lead to activation of regenerative programs. The role of such indirect mechanisms to effectuate therapeutic benefits is suggested by evidence that after stem cell administration and clearance from delivery sites in tissue and organs, regeneration processes nevertheless persist and arise from endogenous tissues. The “paracrine hypothesis” of stem cell regenerative activity has created a paradigm shift by which clinical applications based on exosomes secreted by the stem cells may prove superior, or provide distinct advantages, when compared to transplant and delivery of stem cells themselves. Stem cell-derived exosomes have been identified and isolated from supernatants of several cell types with demonstrated therapeutic potential, including mesenchymal stromal (MSC), (bone marrow stem cells) mononuclear (MNC), immune cells (dendritic and CD34+) and human neural stem cells (hNSCs). In the context of heart disease, human cardiosphere derived cells (CDCs) are known to improve myocardium and vasculature. Stem cell-derived exosomes, including those produced by CDCs, may provide a potent and rich source for developing “cell-free” therapies.

Exosome-based, “cell-free” therapies, in contrast to cell therapy, provide distinct advantages in regenerative medicine. Generally, their production under defined conditions allows for easier manufacture and scale-up opportunity. They further obviate safety issues as non-viable entities, with reduced or non-existent immunogenic or tumorigenic potential. For example, manufacture of exosomes is akin to conventional biopharmacological product manufacture, allowing for standardization in production and quality control for dosage and biological activity testing. The durability of exosomes in culture allows for the acquisition of large quantities of exosomes through their collection from a culture medium in which the exosomes are secreted over periods of time. In addition, exosome encapsulation of bioactive components in lipid vesicles allows protection of contents from degradation in vivo, thereby potentially negating obstacles often associated with delivery of soluble molecules such as cytokines, growth factors, transcription factors and RNAs. Further, stem cell-derived exosomes are likely to be less immunogenic than parental cells, as a result of a lower content of membrane-bound proteins, including MHC complex molecules. Replacing the administration of live cells with their secreted exosomes, mitigates many of the safety concerns and limitations associated with the transplantation of viable replicating cells.

General Features of Exosomes.

Secreted by a wide range of cell types, exosomes are lipid bilayer vesicles that are enriched in a variety of biological factors, including cytokines, growth factors, transcription factors, and coding and non-coding nucleic acids. Exosomes are found in blood, urine, amniotic fluid, interstitial and extracellular spaces. These exocytosed vesicles of endosomal origin can range in size between 30-300 nm, including sizes of 40-100 nm, and possess a cup-shaped morphology, as revealed by electron microscopy. Their initial formation begins with inward budding of the cell membrane to form endosomes, which is followed by invagination of the limiting membrane of late endosomes to form multivesicular bodies (MVB). Fusion of the MVB with the plasma membrane results in the release of the internal vesicles to the extracellular space, through the formation of vesicles thereafter known as exosomes.

As described, the “cargo” contents of exosomes reflect their parental cellular origin, as containing distinct subsets of biological factors in connection with their parent cellular origin, including the cell regulatory state when formed. Exosomes contain a biological milieu of different proteins, including cytokines and growth factors, coding and noncoding RNA molecules, all necessarily derived from their parental cells. In addition to containing a rich array of cytosolic derivatives, exosomes further express the extracellular domain of membrane-bound receptors at the surface of the membrane.

It is now well-established that exosomes are involved in intercellular communication between different cell types, but much remains to be discovered in regard to the mechanisms of their production within parental cells of origin and effects on target recipient cells. Exosomes have been reported to be involved in numerous cellular, tissue and physiological processes, including immune modulating processes, angiogenesis, migration of endothelial cells in connection with tumor growth, or reducing damage in ischemia reperfusion injury. Because exosomes contain cargo contents reflecting the parental cell type and its cellular regulatory state at time of production, the resulting composition of exosomes play a critical role in determining their function. Of critical scientific interest in establishing whether exosomes secreted by cells, such as cardiosphere-derived cells (CDCs), are capable of reproducing the therapeutic benefits of their parental cells, or possible, are indispensable in effectuating such therapeutic benefits

The described encapsulation and formation processes necessarily create heterogeneity in exosome compositions based on parental cellular origin and regulatory state at time of formation. Nevertheless, generic budding formation and release mechanisms establish a common set of features as a consequence of their origin, such as endosome-associated proteins (e.g., Rab GTPase, SNAREs, Annexins, and flotillin), proteins that are known to cluster into microdomains at the plasma membrane or at endosomes (four transmembrane domain tetraspanins, e.g., CD63, CD81, CD82, CD53, and CD37), lipid raft associated proteins (e.g., glycosylphosphatidylinositol-anchored proteins and flotillin), cholesterol, sphingomyelin, and hexosylceramides, as examples.

In addition to these core components reflecting their vesicle origin, a critical property of exosomes is a demonstrated capability to contain both mRNA and microRNA associated with signaling processes, with both cargo mRNA being capable of translation in recipient cells, or microRNA functionally degrading target mRNA in recipient cells. Other noncoding RNAs, capable for influencing gene expression, may also be present in exosomes. While the processes governing the selective incorporation of mRNA or microRNA populations into exosomes is not entirely understood, it is clearly that RNA molecules are selectively, not randomly incorporated into exosomes, as revealed by studies report enrichment of exosome cargo RNAs when compared to the RNA profiles of the originating cells. Given the growing understanding of how such RNA molecules play a role in disease pathogenesis and regenerative processes, the presence of RNA molecules in exosomes and apparent potency in effecting target recipient cells suggests critical features that can be deployed in therapeutic approaches.

Importantly, the natural bilayer membrane encapsulation of exosomes provides a protected and controlled internal microenvironment that allows cargo contents to persist or migrate in the bloodstream or within tissues without degradation. Their release into the extracellular environment, allows for interaction with recipient cells via adhesion to the cell surface mediated by lipid-ligand receptor interactions, internalization via endocytic uptake, or by direct fusion of the vesicles and cell membrane. These processes lead to the release of exosome cargo content into the target cell. The net result of exosome-cell interactions is modulation of genetic pathways in the target recipient cell, as induced through any of several different mechanisms including antigen presentation, the transfer of transcription factors, cytokines, growth factors, nucleic acid such as mRNA and microRNAs. In the stem cell context, embryonic stem cell (ESC)-derived exosomes have been demonstrated to shuttle/transfer mRNA and proteins to hematopoietic progenitors. Other studies have shown that adult stem cell-derived exosomes also shuttle selected patterns of mRNA, microRNA and pre-microRNA associated with several cellular functions involved in the control of transcription, proliferation and cell immune regulation.

Isolation and Preparation of Exosomes.

Exosome isolation relies on exploiting their generic biochemical and biophysical features for separation and analysis. For example, differential ultracentrifugation has become a leading technique wherein secreted exosomes are isolated from the supernatants of cultured cells. This approach allows for separation of exosomes from nonmembranous particles, by exploiting their relatively low buoyant density. Size exclusion allows for their separation from biochemically similar, but biophysically different microvesicles, which possess larger diameters of up to 1,000 nm. Differences in floatation velocity further allows for separation of differentially sized exosomes. In general, exosome sizes will possess a diameter ranging from 30-300 nm, including sizes of 40-100 nm. Further purification may rely on specific properties of the particular exosomes of interest. This includes, for example, use of immunoadsorption with a protein of interest to select specific vesicles with exoplasmic or outward orientations.

Among current methods (differential centrifugation, discontinuous density gradients, immunoaffinity, ultrafiltration and high performance liquid chromatography (HPLC), differential ultracentrifugation is the most commonly used for exosome isolation. This technique utilizes increasing centrifugal force from 2000×g to 10,000×g to separate the medium- and larger-sized particles and cell debris from the exosome pellet at 100,000×g. Centrifugation alone allows for significant separation/collection of exosomes from a conditioned medium, although it is insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/ml) or application of a discrete sugar cushion in preparation.

Importantly, ultrafiltration can be used to purify exosomes without compromising their biological activity. Membranes with different pore sizes—such as 100 kDa molecular weight cut-off (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to homogeneously sized particles and preserve their biological activity as the preparation is maintained at a physiological pH and salt concentration.

Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FlFFF) is an elution-based technique that is used to separate and characterize macromolecules (e.g., proteins) and nano- to micro-sized particles (e.g., organelles and cells) and which has been successfully applied to fractionate exosomes from culture media.

Beyond these techniques relying on general biochemical and biophysical features, focused techniques may be applied to isolated specific exosomes of interest. This includes relying on antibody immunoaffinity to recognizing certain exosome-associated antigens. Conjugation to magnetic beads, chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

Exosome-Based Therapies.

A chief goal of developing exosome-based therapy is the creation of “cell-free” therapies, wherein the benefits of cell therapeutics can be provided with reduced risks or in scenarios in which cell therapy would be unavailable.

Without being bound by any particular theory, the Inventors believe that the therapeutic effects of stem cells can be reproduced by exosomes, and are possibly indispensable to such regenerative processes. In fact, focused application of exosomes may actually provide superior results for the following reasons. Firstly, the retention of delivered stem cells has been shown to be short lived. Second, the quantity of local release of exosomes from a stem cell is limited and occurs only as long as the cell is retained. Thirdly, the quantity of exosomes delivered can be much higher (i.e., high dosing of its contents). Fourth, exosomes can be readily taken up by the cells in the local tissue milieu. Fifth, issues of immunogenicity are avoided. Lastly, repeated doses of exosomes is feasible, while impractical/potentially dangerous for stem cells as they impact the microvasculature. The use of cell-based-exosome therapy has the potential to impact directly on the pathology in heart disease and related conditions by reversing the course of the disease, as opposed to palliative or preventive measures. Such approaches focused on bona fide regenerative of diseased or dysfunctional tissue, representing a major therapeutic breakthrough in both direct repair of injured tissue and in generation of support vasculature that ultimate supports the development and homeostasis of regenerated tissue. Such approaches are not addressed by the current pharmacologic tools currently employed in the treatment of this devastating condition.

While stem cell therapy for heart disease and related conditions has long been a promising concept for addressing such issues, they depend highly on successful delivery into the myocardial area of need. General principles from such techniques (e.g., concentration, timing of delivery, and sustained bioavailability) are applicable to exosome-based therapy. However, a key benefit of exosome based therapy is that the central challenges limiting cellular transplants are largely obviated (e.g., cell engraftment of cells and prolonged survival of the transplanted cells). For example, a key limitation of cell delivery is providing a sufficient number of cells to maximize therapeutic effect, such cells being susceptible to clearance and washout. Furthermore, the regenerative effects of delivered cells may further rely on migration and homing mechanisms to potentiate their stem cell activity at the site of injury. Physiological or biochemical barriers may effectively eliminate administered cells moving to sites of repair. Unlike cell therapy, the Inventors believe higher concentrations of biological agents to the local tissue milieu is possible via exosomes, and that repeated administration of such exosomes may maximize tissue regeneration and repair in a manner that would be infeasible for cell therapy.

Generally, exosome based therapy can delivered via a number of routes: intravenous, intracoronary, and intramyocardial. Exosomes, also allow for new delivery routes that were previously infeasible for cell therapy, such as inhalation. Benefits and drawbacks of these various approaches are described below.

Intravenous delivery technique can occur through a peripheral or central venous catheter. As the simplest delivery mode, this techniques avoids the risk of an invasive procedure. However, intravenous may be regarded as a comparatively inefficient and less localized delivery method, as a high percentage of infused cell exosomes may become sequestered in organs such as the lung, liver, or spleen. Such sequestration may results in few or no cellular exosomes reaching coronary circulation or have unintended systemic effects following their distribution. Exosomes reaching the site of injury may also face additional obstacles when migrating across or effectuating signaling across cells in the arterial or capillary wall. Importantly, this route is unlikely to exist as an option for patients with occluded arteries, unless there are sufficient routes of collateral coronary artery circulation exist.

By contrast, an approach that may be preferential involves intracoronary cell infusion. As delivered through the central lumen of a balloon catheter positioned in the coronary artery, exosomes can be administered with coronary flow. In some instances, balloon occlusion is used to introduce flow interruption as a means to minimize washout of the therapeutic. A key advantage of the intracoronary approach is selective, local delivery of cells to the myocardial area of interest, thereby limiting risks of systemic administration. Coronary delivery requires that the target myocardium be subtended by a patent coronary artery or identifiable collateral vessel and therefore performed following percutaneous coronary intervention (PCI). In some therapeutic contexts, such as acute myocardial infarct, the relative ease of delivery following standard catheter intervention to re-establish coronary flow is a highly attractive opportunity for intracoronary delivery.

In another approach, direct intramyocardial delivery via injection into the myocardium via a transepicardial or transendocardial entry. While this epicardial approach allows for direct visualization of the infarcted myocardium for accurate targeting of delivery, it requires open-heart surgery. Targeted injections can also be obtained by an endocardial approach, which obviates the need for surgery and has been applied as a stand-alone procedure, but the lack of direct visualization presents some difficulties. Further, existing studies of direct injection into the myocardium may result in delivery only to relatively small myocardial areas, resulting in nonuniform distribution within the recipient heart intramyocardial injection of CSCs would be difficult to achieve clinically on a widespread basis, and a limitation of both epicardial and endocardial approaches is the risk of perforation. Nevertheless, such direct injection techniques can be used in instances wherein transvascular delivery is not possible, such as patients with an ischemic cardiomyopathy and occluded coronary artery.

An alternative intravenous mode may be retrograde coronary sinus delivery. This approach relies on catheter placement into the coronary sinus, inflation of the balloon, and exosome administered by infusion at pressures higher than coronary sinus pressure (e.g., 20 mL), thereby allowing for retrograde perfusion of cells into the myocardium. Like intracoronary delivery, exosomes could be required to migrate across or effectuating their signaling across the arterial or capillary wall.

Biochemical Mechanisms Underlying Therapeutic Effects.

As described, protein, bio-active lipid and nucleic acid “cargo” of exosomes have been demonstrated as inducing significant phenotypic and functional changes in recipient cells. Between same cell types, it has been shown that transfer occurs among dendritic cells, hepatocellular carcinoma cells, and adipocytes. Between different cell types, it has also been demonstrated that exosome mediated transfer occurs from T-cells to antigen-presenting cells, from stem cells to endothelial cells and fibroblasts, from macrophages to breast cancer cells, and from epithelial cells to hepatocytes.

The “paracrine hypothesis” of stem cell regenerative activity as mediated via exosomes is suggested by evidence that after stem cell administration and clearance from delivery sites in tissue and organs, regeneration processes nevertheless persist and arise from endogenous tissues. Precisely what cargo contents are transferred that confer therapeutic benefit, and which cells receive such factors to effectuate repair remains a mystery. In general, what is understood is that release of exosomes into the extracellular environment, allows for interaction with recipient cells via adhesion to the cell surface mediated by lipid-ligand receptor interactions, internalization via endocytic uptake, or by direct fusion of the vesicles and cell membrane. These processes lead to the release of exosome cargo content into the target cell. The net result of exosome-cell interactions is modulation of genetic pathways in the target recipient cell, as induced through any of several different mechanisms including antigen presentation, the transfer of transcription factors, cytokines, growth factors, nucleic acid such as mRNA and microRNAs.

Producing exosomes containing critical cargo contents, either via biologic or synthetic production could possibly reproduce the therapeutic benefits of their parental cells, or possible, are indispensable in effectuating such therapeutic benefits The Inventors have recently established that certain microRNAs are enriched in CDC-derived exosomes when compared to fibroblasts, the latter having been establish as providing no salutary benefit in heart disease and related conditions. The demonstrated capability of exosomes to contain both mRNA and microRNA associated with signaling processes, with both cargo mRNA being capable of translation in recipient cells, or microRNA functionally degrading target mRNA in recipient cells has been described as “shuttle RNA”, and it is suggested that the RNAs and microRNAs enriched in CDCs are vital “shuttle RNA” components potentiating stem cell activity.

Further, while studies focusing on regeneration processes have hinted at the long-term effects of stem cell administration as mediated via exosomes, of compelling interest are the discrete, focused effects, such as the existence of a possible nexus between CDC-derived exosomes, cardioprotection and immune response. It has been suggested that stem cells, such as mesenchymal stem cells (MSCs) secreted exosome factors capable of mediating macrophage response and thereby modulating inflammation. Macrophages (i.e., Mφ) predominantly expressing the killer phenotype are called M1 macrophages (proinflammatory), whereas those involved in tissue repair are called M2 macrophages (healing type). If stem cells, such as CDCs and/or their secreted exosomes, were demonstrated as capable of polarizing Mφ toward M2, the enhancement of healing type macrophages function processes like wound and tissue repair would strongly suggest their use in adjunctive therapies. For example, the relative ease of delivery following standard catheter intervention to re-establish coronary flow represents a highly attractive opportunity for intracoronary delivery of CDC-derived exosomes for their immediate cardioprotective effects.

More specifically, cardiac ischemic injury involves both protective and cytotoxic cell types and an inflammatory cascade proceeds through a canonical series of events: first, an influx of neutrophils and macrophages to clear necrotic debris; later, deposition of extracellular matrix and release of growth factors; and finally, the resolution of inflammation and maturation of the scar through cross-linking of collagen fibers. Thus, inflammation converts necrotic tissue into scar, but the abundance of cytotoxic cells recruited into the myocardium has the potential to exacerbate injury. Despite longstanding appreciation of the detrimental inflammatory consequences of ischemia and reperfusion, clinically-useful interventions targeting this pathway are lacking. Among these heterogeneous cell populations, macrophages are one category of important cell type that may be functionally traced to their site of origin (bone marrow versus yolk sac) and spatial localization (tissue resident versus peripheral, monocyte-derived). In many tissues, including the brain, liver, and lung, resident Mφ confer environmental homeostasis and maintain residency through local proliferation. However, following tissue injury, inflammatory monocytes are recruited to the site of injury, differentiate into Mφ, and proliferate in order to support repair. In the setting of myocardial infarction (MI) and ischemia-reperfusion (IR) injury, monocytes are recruited from bone marrow and splenic reserves in a biphasic manner. The early Ly6C^{hi} population, which is most commonly associated with the M1 proinflammatory Mφ phenotype, is recruited as a result of increased MCP-1/CCR2 chemokine/monocyte receptor interaction and elevated expression of endothelial adhesion molecules. Between days 4-7 post-MI, a late Ly6C^{lo} population, which is most commonly associated with the M2 “healing” phenotype, infiltrates the myocardium. Interestingly, targeted depletion of either population with clodronate liposomes leads to impaired infarct healing. Therefore, a heterogeneous population of Mφ, derived from both cardiac and peripheral inflammatory sources, exists in congruence at the site of injury to support repair. Despite the well-established characterization of M₁ and M₂ subpopulations, Mφ can assume a multitude of activated states in response to microenvironmental cues. In fact, within the adult heart at least four distinct resident Mφ subsets exist under steady-state conditions. Following MI, endogenous cardiac-derived chemotactic signals and danger-associated molecular patterns (DAMPs) are released from the infarcted tissue, promoting the expansion of resident and monocyte-derived Mφ into distinct phenotypes. The resulting microenvironment supports diverse capacities for phagocytosis, antigen presentation, and T-cell activation, while other immune cell types, such as B-cells, may regulate monocyte mobilization to the site of injury.

As described, cardiosphere-derived cells (CDCs) are a unique heart-derived cell type that confer significant functional and structural benefits including reduction of infarct size, improvement of cardiac function, enhanced angiogenesis, and modulation of the inflammatory response post-MI. It is currently unknown whether CDCs are able to confer acute cardioprotection (within 48 hours) following ischemic injury or whether they modify the innate immune response. Here, it is described that administration of CDCs 20 minutes post-IR reduces infarct mass and improves function. Importantly, it is demonstrated that these therapeutic effects are abolished by systemic Mφ depletion and reproduced by adoptive transfer of CDC-primed Mφ. Of great interest is further understanding whether exosomes secreted by cells such as CDCs, are alone capable of reproducing therapeutic benefits of their parental cells, or possibly indispensable in these processes. Confirming the role of exosomes in such processes, including modulation of inflammation, will allow their application in new therapeutic approaches. This includes “cell-free” use in subjects for cardioprotection adjunctive to standard therapy, or in contexts which cellular transplant or administration is unavailable. There is a great need in the art for identifying means by which to deliver the benefits of stem cell regeneration, without resorting to mechanisms involving administration or transplant of the cell themselves.

Described herein are compositions and methods and compositions providing significant benefits in the repair or regeneration of damaged or diseased tissues via “cell-free” methods involving exosomes. Specifically, human cardiosphere-derived cells (CDC)-derived exosomes are demonstrated as effective in reducing scar size and regenerating viable myocardium. Such results confirm that the major benefits of CDC cell therapy are mediated by exosomes, including specific microRNAs identified by the Inventors as enriched in CDCs.

Described herein is a method of modulating inflammation, including selecting a subject afflicted with an inflammatory related disease and/or condition; and administering a composition including a plurality of exosomes to the subject, wherein administration of the composition modulates inflammation in the subject by polarizing an endogenous population of macrophages in the subject. In other embodiments, the inflammatory related disease and/or condition is acute. In other embodiments, the inflammatory related disease and/or condition is chronic. In other embodiments, the inflammatory related disease and/or condition is a heart related disease and/or condition. In other embodiments, the heart related disease and/or condition is myocardial infarct. In other embodiments, the heart related disease and/or condition is atherosclerosis and/or heart failure. In other embodiments, polarizing an endogenous population of macrophages includes appearance of M_{CDC} macrophage phenotype, decreased M1 macrophage phenotype and/or increased M2 macrophage phenotype. In other embodiments, the M_{CDC} macrophage phenotype includes expression of one or more of interleukin-10 (Il10) and interleukin-4ra (Il4ra), M1 macrophage phenotype includes expression of one or more of nitric oxidate synthase (Nos2), tumor necrosis factor (Tnf), interleukin-1 (Il1), and interleukin6 (Il6), and M2 macrophage phenotype includes expression of one or more of arginase 1 (Arg1), interleukin-10 (Il10), and peroxisome proliferator-activated receptor gamma (Pparg). In other embodiments, decreased M1 macrophage phenotype and/or increased M2 macrophage phenotype includes an increase in Arg1/Nos2 ratio in a population of macrophages. In other embodiments, decreased M1 macrophage phenotype and/or increased M2 macrophage phenotype includes a decrease in Ly6C expression in a population of macrophages. In other embodiments, the macrophages are from cardiac tissue, peritoneum, spleen and/or bone marrow. In other embodiments, administering a composition includes 1×10⁸ or more exosomes in a single dose. In other embodiments, administering a composition includes about 1×10⁵ to about 1×10⁸ or more CDCs in a single dose. In another example, the number of administered CDCs includes intracoronary 25 million CDCs per coronary artery (i.e., 75 million CDCs total) as another baseline for exosome dosage quantity. In various embodiments, exosome quantity may be defined by protein quantity, such as dosages including 1-10, 10-25, 25-50, 50-75, 75-100, or 100 or more mg exosome protein. In other embodiments, a single dose is administered multiple times to the subject. In other embodiments, administering a composition consists of one or more of: intra-arterial infusion, intravenous infusion, percutaneous injection, and injection directly into heart tissue. Further examples are found in U.S. application Ser. Nos. 11/666,685, 12/622,143, and 12/622,106, which are herein incorporated by reference. Other examples or embodiments relating to the composition and techniques involving exosomes are presented, in PCT Pub. No. WO 2014/028,493, which is fully incorporated herein by reference.

Further described herein is a method of conferring cardioprotection, including selecting a subject afflicted with myocardial infarct (MI), ischemia/reperfusion (IR), or both and administering a composition including a plurality of exosomes to the subject, wherein the plurality of the exosomes are isolated from cardiosphere-derived cells (CDCs) grown in serum-free media, include one or more exosomes with a diameter of about 90 nm to about 200 nm and are CD81+, CD63+, or both, and further wherein administration of the composition confers cardioprotection by polarizing an endogenous population of macrophages in the subject. In other embodiments, the macrophages are from cardiac tissue, peritoneum, spleen and/or bone marrow. In other embodiments, administering a composition includes 1×10⁸ or more exosomes in a single dose. In other embodiments, a single dose is administered multiple times to the subject.

In other embodiments, administering a composition consists of one or more of: intra-arterial infusion, intravenous infusion, percutaneous injection, and injection directly into heart tissue. In other embodiments, administering a composition including a plurality of exosomes to the subject is adjunctive to standard therapy. In other embodiments, administering a composition is less than 1 hour after reperfusion. In other embodiments, conferring cardioprotection reduces infarct size.

Further described herein is a method, including providing a population of cells including stem cells, progenitors, and/or precursor cells, and isolating a plurality of exosomes from the population of cells, wherein the plurality of exosomes include one or more exosomes with a diameter of about 90 nm to 200 nm, are CD81+, CD63+, or both, and are about 2-5 kDa. In other embodiments, the stem cells, progenitors, and/or precursor cells include cardiosphere-derived cells (CDCs) grown in serum-free media, and are confluent when isolating the plurality of exosomes. In other embodiments, the plurality of exosomes include one or more exosomes including one or more microRNAs selected from the group consisting of: miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In other embodiments, isolating the plurality of exosomes includes precipitation, centrifugation, filtration, immuno-separation, and/or flow fractionation.

Also described herein is a composition produced by the method including providing a population of cells including stem cells, progenitors, and/or precursor cells, and isolating a plurality of exosomes from the population of cells, wherein the plurality of exosomes include one or more exosomes with a diameter of about 90 nm to 200 nm, are CD81+, CD63+, or both, and are about 2-5 kDa. Further described herein is a the stem cells, progenitors, and/or precursor cells include cardiosphere-derived cells (CDCs) grown in serum-free media, and are confluent when isolating the plurality of exosomes.

Further described herein is an in vitro method of altering a cell, including providing a plurality of exosomes and adding to a starting cell type, the plurality of exosomes, wherein adhesion between one or more exosomes in the plurality of exosomes and the starting cell type is capable of altering one or more properties of the starting cell type, and generating a converted cell type. In other embodiments, the plurality of exosomes are derived from stem cells, progenitors, and/or precursor cells. In other embodiments, the stem cells, progenitors, and/or precursor cells include cardiosphere-derived cells (CDCs). In other embodiments, the stem cells, progenitors, and/or precursor cells include endothelial precursor cells (EPCs) and/or mesenchymal stem cells (MSCs). In other embodiments, the starting cell type is a fibroblast.

Also described herein is a quantity of converted cells made by the in vitro method of altering a cell, including providing a plurality of exosomes and adding to a starting cell type, the plurality of exosomes, wherein adhesion between one or more exosomes in the plurality of exosomes and the starting cell type is capable of altering one or more properties of the starting cell type, and generating a converted cell type.

Further described herein is an in vivo method of altering a cell, including selecting a subject and administering a composition including a plurality of exosomes to the subject, wherein adhesion between one or more exosomes in the plurality of exosomes and a starting cell type is capable of altering one or more properties of the starting cell type, thereby generating a converted cell type in vivo. In other embodiments, the plurality of exosomes are derived from stem cells, progenitors, and/or precursor cells. In other embodiments, the stem cells, progenitors, and/or precursor cells include cardiosphere-derived cells (CDCs). In other embodiments, the stem cells, progenitors, and/or precursor cells include endothelial precursor cells (EPCs) and/or mesenchymal stem cells (MSCs). In other embodiments, the starting cell type is a fibroblast.

Further described herein is method of modulating inflammation, including selecting a subject in need of treatment for inflammatory related disease and/or condition and administering a composition including a plurality of exosomes to the subject, wherein administration of the composition modulates inflammation in the subject. In other embodiments, the inflammatory related disease and/or condition is acute. In other embodiments, the inflammatory related disease and/or condition is chronic. In other embodiments, the inflammatory related disease and/or condition is a heart related disease and/or condition. In other embodiments, the heart related disease and/or condition is myocardial infarct. In other embodiments, the heart related disease and/or condition is atherosclerosis and/or heart failure. In other embodiments, modulating inflammation in the subject includes appearance of M_{CDC} macrophage phenotype, decreased M1 macrophage phenotype and/or increased M2 macrophage phenotype. In other embodiments, the M_{CDC} macrophage phenotype includes expression of one or more of interleukin-10 (Il10) and interleukin-4ra (Il4ra), M1 macrophage phenotype includes expression of one or more of nitric oxidate synthase (Nos2), tumor necrosis factor (Tnf), interleukin-1 (Il1), and interleukin6 (Il6), and M2 macrophage phenotype includes expression of one or more of arginase 1 (Arg1), interleukin-10 (Il10), and peroxisome proliferator-activated receptor gamma (Pparg). In other embodiments, decreased M1 macrophage phenotype and/or increased M2 macrophage phenotype includes an increase in Arg1/Nos2 ratio in a population of macrophages. In other embodiments, decreased M1 macrophage phenotype and/or increased M2 macrophage phenotype includes a decrease in Ly6C expression in a population of macrophages. In other embodiments, the macrophages are from cardiac, peritoneal, spleen and/or bone marrow. In other embodiments, administering a composition includes 1×10⁸ or more exosomes in a single dose. In other embodiments, administering a composition includes about 1×10⁵ to about 1×10⁸ or more CDCs in a single dose. In another example, the number of administered CDCs includes intracoronary 25 million CDCs per coronary artery (i.e., 75 million CDCs total) as another baseline for exosome dosage quantity. In various embodiments, the numbers of CDCs includes 1×10⁵, 1×10⁶, 1×10⁷, 1×10⁸, 1×10⁹ CDCs in a single dose as another baseline for exosome dosage quantity. In certain instances, this may be prorated to body weight (range 100,000-1M CDCs/kg body weight total CDC dose). In other embodiments, a single dose is administered multiple times to the subject. In other embodiments, administering a composition consists of one or more of: intra-arterial infusion, intravenous infusion, percutaneous injection, and injection directly into heart tissue. In other embodiments, one or more exosomes in the plurality of exosomes are CD63+, CD81+, or both. In other embodiments, one or more exosomes in the plurality of exosomes have a diameter of about 30 nm to 300 nm. In other embodiments, one or more exosomes in the plurality of exosomes have a diameter of about 90 nm to 200 nm. In other embodiments, the plurality of exosomes are derived from stem cells, progenitors, and/or precursor cells. In other embodiments, the stem cells, progenitors, and/or precursor cells include cardiosphere-derived cells (CDCs). In other embodiments, the stem cells, progenitors, and/or precursor cells include endothelial precursor cells (EPCs) and/or mesenchymal stem cells (MSCs). In other embodiments, the plurality of exosomes include a protein. In other embodiments, the plurality of exosomes includes a lipid. In other embodiments, administering a composition including a plurality of exosomes to the subject is adjunctive to standard therapy.

Described herein is a composition including a plurality of exosomes. In certain embodiments, the plurality of exosomes are generated by a method including providing a population of cells, and isolating a plurality of exosomes from the population of cells.

In various embodiments, the cells are stem cells, progenitors and/or precursors. In other embodiments, the stem cells, progenitors and/or precursors are cardiosphere-derived cells (CDCs). In other embodiments, the stem cells, progenitors and/or precursors are pluripotent stem cells (pSCs), such as embryonic stem cells (ESCs) and induced pluripotent stem cells (iPSCs) derived from any one of various somatic sources in the body such as fibroblasts, blood and hematopoietic stem cells (hSCs), immune cells, bone and bone marrow, neural tissue, among others. In other embodiments, the stem cells, progenitors and/or precursors include hSCs, mesenchymal stem cells (MSCs), or endothelial precursor cells (EPCs). In various embodiments, the cells are stem cells, progenitors and/or precursors derived from human biopsy tissue. In various embodiments, the cells are stem cells, progenitors and/or precursors are a primary culture. In various embodiments, the cells are stem cells, progenitors and/or precursors which may constitute a cell line capable of serial passaging.

In various embodiments, the plurality of exosomes are isolated from the supernatants of the population of cells. This includes, for example, exosomes secreted into media as conditioned by a population of cells in culture, further including cell lines capable of serial passaging. In certain embodiments, the cells are cultured in a serum-free media. In certain embodiments, the cells in culture are grown to 10, 20, 30, 40, 50, 60, 70, 80, 90, or 90% or more confluency when exosomes are isolated. In certain embodiments, the population of cells has been genetically manipulated. This includes, for example, knockout (KO) or transgenic (TG) cell lines, wherein an endogenous gene has been removed and/or an exogenous introduced in a stable, persistent manner. In certain embodiments, the cells are genetically modified to express endothelial nitric oxide synthase (eNOS), vascular endothelial growth factor (VEGF), SDF-1 (stromal derived factor), IGF-1 (insulin-like growth factor 1), HGF (hepatocyte growth factor). This further includes transient knockdown of one or more genes and associated coding and non-coding transcripts within the population of cells, via any number of methods known in the art, such as introduction of dsRNA, siRNA, microRNA, etc. This further includes transient expression of one or more genes and associated coding and non-coding transcripts within the population of cells, via any number of methods known in the art, such as introduction of a vector, plasmid, artificial plasmid, replicative and/or non-replicative virus, etc. In other embodiments, the population of cells has been altered by exposure to environmental conditions (e.g., hypoxia), small molecule addition, presence/absence of exogenous factors (e.g., growth factors, cytokines) at the time, or substantially contemporaneous with, isolating the plurality of exosomes in a manner altering the regulatory state of the cell. For example, one may add a differentiation agent to a population of stem cells, progenitors and/or precursors in order to promote partial or full differentiation of the cell, and thereafter derive a plurality of exosomes. In various embodiments, altering the regulatory state of the cell changes composition of one or more exosomes in the plurality of exosomes.

In various embodiments, the plurality of exosomes includes one or more exosomes that are about 10 nm to about 250 nm in diameter, including those about 10 nm to about 15 nm, about 15 nm to about 20 nm, about 20 nm to about 25 nm, about 25 nm to about 30 nm, about 30 nm to about 35 nm, about 35 nm to about 40 nm, about 40 nm to about 50 nm, about 50 nm to about 60 nm3 about 60 nm to about 70 nm, about 70 nm to about 80 nm, about 80 nm to about 90 nm, about 90 nm to about 95 nm, about 95 nm to about 100 nm, about 100 nm to about 105 nm, about 105 nm to about 110 nm, about 110 nm to about 115 nm, about 115 nm to about 120 nm, about 120 nm to about 125 nm, about 125 nm to about 130 nm, about 130 nm to about 135 nm, about 135 nm to about 140 nm, about 140 nm to about 145 nm, about 145 nm to about 150 nm, about 150 to about 200 nm, about 200 nm to about 250 nm, about 250 nm or more.

In various embodiments, the plurality of exosomes includes one or more exosomes expressing a biomarker. In certain embodiments, the biomarkers are tetraspanins. In other embodiments, the tetraspanins are one or more selected from the group including CD63, CD81, CD82, CD53, and CD37. In other embodiments, the exosomes express one or more lipid raft associated proteins (e.g., glycosylphosphatidylinositol-anchored proteins and flotillin), cholesterol, sphingomyelin, and/or hexosylceramides.

In several embodiments, the plurality of exosomes includes one or more exosomes containing a biological protein. In various embodiments, the biological protein includes transcription factors, cytokines, growth factors, and similar proteins capable of modulating signaling pathways in a target cell. In various embodiments, the biological protein is capable of facilitating regeneration and/or improved function of a tissue. In other embodiments, the biological protein is capable of modulating a pathway related to vasodilation, such as prostacyclin and nitric oxide, and/or vasoconstrictors such as thromboxane and endothelin-1 (ET-1). In various embodiments, the biological protein is capable of modulating pathways related to Irak1, Traf6, toll-like receptor (TLR) signaling pathway, NOX-4, SMAD-4, and/or TGF-β. In other embodiments, the biological protein is capable of mediating M1- and/or M2-like immune responses in macrophages, which may further be described as macrophage polarization. For example, this includes gene expression changes in Arg1, Il4ra, Nos2, Il-10, Nfkb1, Tnf, and Vegfa.

In various embodiments, M1 phenotype for Mφ can be described by marker expression, such as Ly6C^{hi}, whereas M2 phenotype can be described by marker expression of Ly6C^{lo}. In other embodiments, macrophage polarization can include increased or decreased of the numbers of Mφ expressing CD45⁺, CD68⁺, or both. In other embodiments, macrophage polarization can include reduced M1-type proinflammatory cytokine expression of one or more of Nos2, Tnf, Il1b, and Il6, elevated M2-type expression of one or more of Arg1, Il10, and Pparg. In other embodiments, macrophage polarization can include changes in ratio of protein expression of Nos2 and Arg1 in Mφ, for example M₂ Mφ may exhibit elevated Arg1/Nos2 ratio, optionally including Lyve-1, and p50 expression, and M₁ Mφ may exhibit reduced Arg1/Nos2 ratio, as well as elevated phospho-p65 expression.

Alternatively, the biological protein is capable of altering Mφ response such as elevated expression of Il10, expression of an Arg1/Nos2 ratio between M₁ and M₂, elevated Lyve-1 relative to naive Mφ low phospho-p65, and low p50 expression. In other embodiments, Mφ express one or more of CD68, CD80, CD86, CD11b, CD45, and FSC. In various embodiments, the biological protein is capable of Mφ response including some or all of the above mentioned features. In various embodiments, the Mφ are from cardiac, peritoneal, spleen and/or bone marrow-derived sources.

In other embodiments, the biological protein related to exosome formation and packaging of cytosolic proteins such as Hsp70, Hsp90, 14-3-3 epsilon, PKM2, GW182 and AGO2. In certain embodiments, the exosomes express CD63, HSP70, CD105 or combinations thereof. In other embodiments, the exosomes do not express CD9 or CD81, or express neither. For example, plurality of exosomes can include one or more exosomes that are CD63+, HSP+, CD105+, CD9−, and CD81−.

In other embodiments, the plurality of exosomes includes one or more exosomes containing a signaling lipid. This includes ceramide and derivatives. In other embodiments, the plurality of exosomes includes one or more exosomes containing a coding and/or non-coding nucleic acid.

In several embodiments, the plurality of exosomes includes one or more exosomes containing microRNAs. In various embodiments, these microRNAs can include miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In several embodiments, the plurality of exosomes includes one or more exosomes enriched in at least one of miR-146a, miR-22, miR-24. Enrichment can be measured by, for example, comparing the amount of one or more of the described microRNAs when derived from cells providing salutary benefit in a therapeutic setting (e.g., cardiosphere-derived cells (CDCs) compared to cells that do not provide such a salutary benefit (e.g., fibroblasts). Enrichment may also be measured in absolute or relative quantities, such as when compared to a standardized dilution series.

In other embodiments, the plurality of exosomes can include one or more exosomes containing microRNAs. This includes various microRNAs known in the art, such as miR-23a, miR-23b, miR-24, miR-26a, miR27-a, miR-30c, let-7e, mir-19b, miR-125b, mir-27b, let-7a, miR-19a, let-7c, miR-140-3p, miR-125a-5p, miR-132, miR-150, miR-155, mir-210, let-7b, miR-24, miR-423-5p, miR-22, let-7f, and/or miR-146a.

In other embodiments, the plurality of exosomes can include one or more exosomes containing microRNAs. This includes various microRNAs known in the art, such as miR-17, miR-21, miR-92, miR92a, miR-29, miR-29a, miR-29b, miR-29c, miR-34, mi-R34a, miR-150, miR-451, miR-145, miR-143, miR-144, miR-193a-3p, miR-133a, miR-155, miR-181a, miR-214, miR-199b, miR-199a, miR-210, miR-126, miR-378, miR-363 and miR-30b, and miR-499. Other microRNAs known in the art include miR-92, miR-17, miR-21, miR-92, miR92a, miR-29, miR-29a, miR-29b, miR-29c, miR-34, mi-R34a, miR-150, miR-451, miR-145, miR-143, miR-144, miR-193a-3p, miR-133a, miR-155, miR-181a, miR-214, miR-199b, miR-199a, miR-126, miR-378, miR-363 and miR-30b, and/or miR-499.

In several embodiments, isolating a plurality of exosomes from the population of cells includes centrifugation of the cells and/or media conditioned by the cells. In several embodiments, ultracentrifugation is used. In several embodiments, isolating a plurality of exosomes from the population of cells is via size-exclusion filtration. In other embodiments, isolating a plurality of exosomes from the population of cells includes use of discontinuous density gradients, immunoaffinity, ultrafiltration and/or high performance liquid chromatography (HPLC).

In certain embodiments, differential ultracentrifugation includes using centrifugal force from 1000-2000×g, 2000-3000×g, 3000-4000×g, 4000-5000×g, 5000×g-6000×g, 6000-7000×g, 7000-8000×g, 8000-9000×g, 9000-10,000×g, to 10,000×g or more to separate larger-sized particles from a plurality of exosomes derived from the cells.

In other embodiments, isolating a plurality of exosomes from the population of cells includes use of filtration or ultrafiltration. In certain embodiments, a size exclusion membrane with different pore sizes is used. For example, a size exclusion membrane can include use of a filter with a pore size of 0.1-0.5 μM, 0.5-1.0 μM, 1-2.5 μM, 2.5-5 μM, 5 or more μM. In certain embodiments, the pore size is about 0.2 μM. In certain embodiments, filtration or ultrafiltration includes size exclusion ranging from 100-500 daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In other embodiments, filtration or ultrafiltration includes size exclusion includes use of hollow fiber membranes capable of isolating particles ranging from 100-500 daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In other embodiments, a molecular weight cut-off (MWCO) gel filtration capable of isolating particles ranging from 100-500 daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In various embodiments, such systems are used in combination with variable fluid flow systems.

In other embodiments, isolating a plurality of exosomes from the population of cells includes use of tangential flow filtration (TFF) systems are used purify and/or concentrate the exosome fractions. In other embodiments, isolating a plurality of exosomes from the population of cells includes use of (HPLC) can also be used to purify exosomes to homogeneously sized particles. In various embodiments, density gradients as used, such as centrifugation in a sucrose density gradient or application of a discrete sugar cushion in preparation.

In other embodiments, isolating a plurality of exosomes from the population of cells includes use of a precipitation reagent. For example, a precipitation reagent, ExoQuick®, can be added to conditioned cell media to quickly and rapidly precipitate a population of exosomes. In other embodiments, isolating a plurality of exosomes from the population of cells includes use of volume-excluding polymers (e.g., polyethylene glycols (PEGs)) are used. In another embodiment, isolating a plurality of exosomes from the population of cells includes use of flow field-flow fractionation (FlFFF), an elution-based technique.

In certain embodiments, isolating a plurality of exosomes from the population of cells includes use of one or more capture agents to isolate one or more exosomes possessing specific biomarkers or containing particular biological molecules. In one embodiment, one or more capture agents include at least one antibody. For example, antibody immunoaffinity recognizing exosome-associated antigens is used to capture specific exosomes. In other embodiments, the at least one antibody are conjugated to a fixed surface, such as magnetic beads, chromatography matrices, plates or microfluidic devices, thereby allowing isolation of the specific exosome populations of interest. In other embodiments, isolating a plurality of exosomes from the population of cells includes use of one or more capture agents that is not an antibody. This includes, for example, use of a “bait” molecule presenting an antigenic feature complementary to a corresponding molecule of interest on the exosome surface, such as a receptor or other coupling molecule. In one embodiment, the non-antibody capture agent is a lectin capable of binding to polysaccharide residues on the exosome surface.

In various embodiments, the CDCs are mammalian. In other embodiments, the CDCs are human. As disclosed above, in some embodiments, synthetic exosomes are generated, which can be isolated by similar mechanisms as those above. In various embodiments, the composition that is a plurality of exosomes is a pharmaceutical composition further including a pharmaceutically acceptable carrier.

In various embodiments, the plurality of exosomes range in size from 30 to 300 nm. In various embodiments, the plurality of exosomes range in size from 40 to 100 nm. In certain embodiments, the plurality of exosomes is cardiosphere-derived cell (CDC) exosomes. In certain embodiments, the plurality of exosomes includes one or more exosomes that are CD63+, CD105+, or both. In various embodiments, the exosomes include microRNAs miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In other embodiments, the exosomes are 2-5 kDa, such as 3 kDa. Other examples or embodiments relating to the composition and techniques involving exosomes are presented, in PCT Pub. No. WO 2014/028,493, which is fully incorporated herein by reference.

Described herein is a method for treatment including, selecting a subject in need of treatment, administering a composition including a plurality of exosomes to the individual, wherein administration of the composition treat the subject. In certain embodiments, the subject is in need to treatment for a disease and/or condition involving tissue damage or dysfunction. In other embodiments, the disease and/or condition involving tissue damage or dysfunction is pulmonary disease. In other embodiments, the disease and/or condition involving tissue damage or dysfunction is heart disease. In other embodiments, the plurality of exosomes includes exosomes including one or more microRNAs.

In certain embodiments, the plurality of exosomes is generated by a method including providing a population of cells, and isolating a plurality of exosomes from the population of cells. In various embodiments, the cells are stem cells, progenitors and/or precursors. In other embodiments, the stem cells, progenitors and/or precursors are cardiosphere-derived cells (CDCs). In other embodiments, the stem cells, progenitors and/or precursors are pluripotent stem cells (pSCs), such as embryonic stem cells (ESCs) and induced pluripotent stem cells (iPSCs) derived from any one of various somatic sources in the body such as fibroblasts, blood and hematopoietic stem cells (hSCs), immune cells, bone and bone marrow, neural tissue, among others. In other embodiments, the stem cells, progenitors and/or precursors include hSCs, mesenchymal stem cells (MSCs), or endothelial precursor cells (EPCs). In various embodiments, the cells are stem cells, progenitors and/or precursors derived from human biopsy tissue. In various embodiments, the cells are stem cells, progenitors and/or precursors are a primary culture. In various embodiments, the cells are stem cells, progenitors and/or precursors which may constitute a cell line capable of serial passaging. In certain embodiments, the exosomes are synthetic.

In various embodiments, the plurality of exosomes is derived from cardiosphere-derived cells (CDCs). In other embodiments, the plurality of exosomes includes exosomes including one or more biological molecules. In other embodiments, the plurality of exosomes including exosomes enriched for one or more biological molecules when derived from CDCs compared to exosome derived from non-CDC sources. In various embodiments, the one or more biological molecules are proteins, growth factors, cytokines, transcription factors and/or morphogenic factors. In other embodiments, the plurality of exosomes including exosomes enriched for one or more biological molecules includes microRNAs, further including microRNAs that are enriched when derived from CDCs compared to exosome derived from non-CDC sources. In various embodiments, these microRNAs can include miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In several embodiments, the plurality of exosomes includes one or more exosomes enriched in at least one of miR-146a, miR-22, miR-24.

In various embodiments, the CDCs are mammalian. In other embodiments, the CDCs are human. In certain embodiments, the exosomes are synthetic. In certain embodiments, the synthetic exosomes possess substantially similar content (e.g., microRNAs, biological molecules) as exosomes derived from CDCs.

In various embodiments, administration of the plurality of exosomes alters gene expression in the damaged or dysfunctional tissue, improves viability of the damaged tissue, and/or enhances regeneration or production of new tissue in the individual. In various embodiments, the quantities of exosomes that are administered to achieved these effects range from 1×10⁶ to 1×10⁷, 1×10⁷ to 1×10⁸, 1×10⁸ to 1×10⁹, 1×10⁹ to 1×10¹⁰, 1×10¹⁰ to 1×10¹¹, 1×10¹¹ to 1×10¹², 1×10¹² or more. In other embodiments, the numbers of exosomes is relative to the number of cells used in a clinically relevant dose for a cell-therapy method. For example, it has been demonstrated that 3 mL/3×10⁵ CDCs, is capable of providing therapeutic benefit in intracoronary administration, and therefore, a plurality of exosomes as derived from that number of cells in a clinically relevant dose for a cell-therapy method. In various embodiments, administration can be in repeated doses. In other embodiments, administering a composition includes about 1×10⁵ to about 1×10⁸ or more CDCs in a single dose. In another example, the number of administered CDCs includes intracoronary 25 million CDCs per coronary artery (i.e., 75 million CDCs total) as another baseline for exosome dosage quantity. In various embodiments, exosome quantity may be defined by protein quantity, such as dosages including 1-10, 10-25, 25-50, 50-75, 75-100, or 100 or more mg exosome protein. For example, defining an effective dose range, dosing regimen and route of administration, may be guided by studies using fluorescently labeled exosomes, and measuring target tissue retention, which can be >10×, >50×, or >100× background, as measured 5, 10, 15, 30, or 30 or more min as a screening criterion. In certain embodiments, >100× background measured at 30 mins is a baseline measurement for a low and high dose that is then assess for safety and bioactivity (e.g., using MRI endpoints: scar size, global and regional function). In various embodiments, single doses are compared to two, three, four, four or more sequentially-applied doses. In various embodiments, the repeated or sequentially-applied doses are provided for treatment of an acute disease and/or condition. In various embodiments, the repeated or sequentially-applied doses are provided for treatment of a chronic disease and/or condition. In other embodiments, administration of the plurality of exosomes is adjunctive to standard therapy. For example, in acute myocardial infarct, a plurality of exosomes be may administered following standard catheter intervention to promote cardioprotection and/or regeneration. In various embodiments, administration of the plurality of exosomes may be within about 5, 10, 15, 20, 30, 45, 60 mins after an acute event. In various embodiments, administration of the plurality of exosomes may be within about 1, 2, 3, 4, 5, 6, 7, 8, 9, 10, 11, 12, 13, 14, 15, 16, 17, 18, 19, 20, 21, 22, 23, 24 or more hours after an acute event. In other embodiments, administration may be within about 5, 10, 15, 20, 30, 45, 60, 90, or 120 mins after ischemia-reperfusion (IR).

In various embodiments, administration of exosomes to the subject occurs through any of known techniques in the art. In some embodiments, this includes percutaneous delivery, and/or injection into heart muscle. In other embodiments, myocardial infusion is used, for example, the use of intracoronary catheters. In various embodiments, delivery can be intra-arterial or intravenous. Additional delivery sites include any one or more compartments of the heart, such as myocardium, associated arterial, venous, and/or ventricular locations. In certain embodiments, administration can include delivery to a tissue or organ site that is the same as the site of diseased and/or dysfunctional tissue. In certain embodiments, administration can include delivery to a tissue or organ site that is different from the site or diseased and/or dysfunctional tissue. In certain embodiments, the delivery is via inhalation or oral administration. In various embodiments, administration of exosomes can include combinations of multiple delivery techniques, such as intravenous, intracoronary, and intramyocardial delivery.

In various embodiments, administration of the plurality of exosomes alters gene expression in the damaged or dysfunctional tissue, improves viability of the damaged tissue, and/or enhances regeneration or production of new tissue in the individual. In various embodiments, administration of the exosomes results in functional improvement in the tissue. In certain embodiments, the damaged tissue is pulmonary, arterial or capillary tissue. In several embodiments, the damaged or dysfunctional tissue includes cardiac tissue.

For example, in certain embodiments in which pulmonary, arterial, capillary, or cardiac tissue is damaged or dysfunctional, functional improvement may include increased cardiac output, contractility, ventricular function and/or reduction in arrhythmia (among other functional improvements). For example, this may include a decrease in right ventricle systolic pressure. For other tissues, improved function may be realized as well, such as enhanced cognition in response to treatment of neural damage, improved blood-oxygen transfer in response to treatment of lung damage, improved immune function in response to treatment of damaged immunological-related tissues. In other embodiments, the disease and/or condition involving tissue damage or dysfunction is pulmonary tissue, including pulmonary, arterial or capillary tissue, such as the endothelial lining of distal pulmonary arteries. In other embodiments, the disease and/or condition involving tissue damage or dysfunction is heart disease.

In various embodiments, administration of the plurality of exosomes alters gene expression in the damaged or dysfunctional tissue, improves viability of the damaged tissue, and/or enhances regeneration or production of new tissue in the individual. In various embodiments, administration of the exosomes results in functional improvement in the tissue. In several embodiments, the damaged or dysfunctional tissue includes skeletal muscle tissue.

For example, in certain embodiments in which skeletal muscle tissue is damaged or dysfunctional, functional improvement may include increased contractile strength, improved ability to walk (for example, and increase in the six-minute walk test results), improved ability to stand from a seated position, improved ability to sit from a recumbent or supine position, or improved manual dexterity such as pointing and/or clicking a mouse.

In various embodiments, the damaged or dysfunctional tissue is in need of repair, regeneration, or improved function due to an acute event. Acute events include, but are not limited to, trauma such as laceration, crush or impact injury, shock, loss of blood or oxygen flow, infection, chemical or heat exposure, poison or venom exposure, drug overuse or overexposure, and the like. In certain embodiments, the damaged tissue is pulmonary, arterial or capillary tissue, such as the endothelial lining of distal pulmonary arteries. In other embodiments, the damaged tissue is cardiac tissue and the acute event includes a myocardial infarction. In some embodiments, administration of the exosomes results in an increase in cardiac wall thickness in the area subjected to the infarction.

In other embodiments, is also subject to damage due to chronic disease, such as for example congestive heart failure, including as conditions secondary to diseases such as emphysema, ischemic heart disease, hypertension, valvular heart disease, connective tissue diseases, HIV infection, liver disease, sickle cell disease, dilated cardiomyopathy, infection such as Schistosomiasis, diabetes, and the like. In various embodiments, the administration can be in repeated doses, such as two, three, four, four or more sequentially-applied doses. In various embodiments, the repeated or sequentially-applied doses are provided for treatment of an acute disease and/or condition. In various embodiments, the repeated or sequentially-applied doses are provided for treatment of a chronic disease and/or condition.

Other sources of damage also include, but are not limited to, injury, age-related degeneration, cancer, and infection. In several embodiments, the regenerative cells are from the same tissue type as is in need of repair or regeneration. In several other embodiments, the regenerative cells are from a tissue type other than the tissue in need of repair or regeneration.

In certain embodiments, the method of treatment includes, selecting a subject in need of treatment for a pulmonary disease and/or condition, administering a composition including a plurality of exosomes to the individual, wherein administration of the composition treat the subject. In certain embodiments, the method of treatment includes, selecting a subject in need of treatment for a heart related disease and/or condition, administering a composition including a plurality of exosomes to the individual, wherein administration of the composition treat the subject. In various embodiments, the heart related disease and/or condition includes heart failure. In various embodiments, the plurality of exosomes range in size from 30 to 300 nm. In various embodiments, the plurality of exosomes range in size from 40 to 100 nm. In certain embodiments, the plurality of exosomes is cardiosphere-derived cell (CDC) exosomes. In certain embodiments, the plurality of exosomes includes one or more exosomes that are CD63+, CD105+, or both. In various embodiments, the exosomes include microRNAs miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In other embodiments, the exosomes are 2-5 kDa, such as 3 kDa. In other embodiments, administering a composition includes a dosage of 1×10⁸, 1×10⁸ to 1×10⁹, 1×10⁹ to 1×10¹⁰, 1×10¹⁰ to 1×10¹¹, 1×10¹¹ to 1×10¹², 1×10¹² or more exosomes. In other embodiments, the numbers of exosomes is relative to the number of cells used in a clinically relevant dose for a cell-therapy method. For example, it has been demonstrated that 3 mL/3×10⁵ CDCs, is capable of providing therapeutic benefit in intracoronary administration, and therefore, a plurality of exosomes as derived from that number of cells in a clinically relevant dose for a cell-therapy method. In various embodiments, administration can be in repeated doses. In other embodiments, administering a composition includes about 1×10⁵ to about 1×10⁸ or more CDCs in a single dose. In another example, the number of administered CDCs includes intracoronary 25 million CDCs per coronary artery (i.e., 75 million CDCs total) as another baseline for exosome dosage quantity. In various embodiments, exosome quantity may be defined by protein quantity, such as dosages including 1-10, 10-25, 25-50, 50-75, 75-100, or 100 or more mg exosome protein. In various embodiments, administering a composition includes multiple dosages of the exosomes. In various embodiments, the repeated or sequentially-applied doses are provided for treatment of an acute disease and/or condition. In various embodiments, the repeated or sequentially-applied doses are provided for treatment of a chronic disease and/or condition. In other embodiments, administering a composition includes myocardial infusion. In other embodiments, administering a composition includes use of an intracoronary catheter. In other embodiments, administration of a composition includes intra-arterial infusion. In other embodiments, administration of a composition includes intravenous infusion. In other embodiments, administering a composition includes percutaneous injection. In other embodiments, administering a composition includes injection into heart muscle. In other embodiments, administration of a composition includes inhalation. In other embodiments, exosome therapy is provided in combination with standard therapy for a disease and/or condition. This may include co-administration of the exosomes with a therapeutic agent or administration adjunctive to standard therapy such as a surgical procedure. In other embodiments, administration may be within about 5, 10, 15, 20, 30, 45, 60, 90, or 120 mins after ischemia-reperfusion (IR).

Described herein is a method of modulating inflammation, including selecting a subject in need of treatment for inflammatory related disease and/or condition; and administering a composition including a plurality of exosomes to the subject, wherein the administration of the composition modulates inflammation in the subject. In other embodiments, the inflammatory related disease and/or condition is acute. In other embodiments, the inflammatory related disease and/or condition is chronic. In other embodiments, the inflammatory related disease and/or condition is a heart related disease and/or condition. In other embodiments, the heart related disease and/or condition is myocardial infarct. In other embodiments, the heart related disease and/or condition is atherosclerosis and/or heart failure. In other embodiments, modulating inflammation in the subject includes decreased M1-like macrophage phenotype and/or elevated M2-like macrophage phenotype. In various embodiments, M1 phenotype for Mφ can be described by marker expression, such as Ly6C^{hi}, whereas M2 phenotype can be described by marker expression of Ly6C^{lo}. In other embodiments, macrophage polarization can include increased or decreased of the numbers of Mφ expressing CD45⁺, CD68⁺, or both. In other embodiments, macrophage polarization can include reduced M1-type proinflammatory cytokine expression of one or more of Nos2, Tnf, Il1b, and Il6, elevated M2-type expression of one or more of Arg1, Il10, and Pparg. In other embodiments, macrophage polarization can include changes in ratio of protein expression of Nos2 and Arg1 in Mφ, for example M₂ Mφ may exhibit elevated Arg1/Nos2 ratio, optionally including Lyve-1, and p50 expression, and M₁ Mφ may exhibit reduced Arg1/Nos2 ratio, as well as elevated phospho-p65 expression. Alternatively, modulating inflammation may include altering Mφ response such as elevated expression of Il10, expression of an Arg1/Nos2 ratio between M₁ and M₂, elevated Lyve-1 relative to naive Mφ low phospho-p65, and low p50 expression. In other embodiments, Mφ express one or more of CD68, CD80, CD86, CD11b, CD45, and FSC. In various embodiments, the biological protein is capable of Mφ response including some or all of the above mentioned features. In various embodiments, the Mφ are from cardiac, peritoneal, spleen and/or bone marrow-derived sources.

Described herein is an in vitro method of altering a cell, including providing a plurality of exosomes, and adding to a starting cell type, the plurality of exosomes, wherein adhesion between one or more exosomes in the plurality of exosomes and the starting cell type is capable of altering one or more properties of the starting cell type, and generating a converted cell type. In other embodiments, the plurality of exosomes includes a nucleic acid. In other embodiments, the nucleic acid includes a ribonucleic acid (RNA). In other embodiments, the RNA includes microRNA. In other embodiments, the one or more exosomes in the plurality of exosomes includes one or more microRNAs selected from the group consisting of: miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In other embodiments, the one or more exosomes in the plurality of exosomes includes miR-146a, miR22, and miR-24. In other embodiments, the one or more exosomes in the plurality of exosomes is CD63+, CD105+, or both. In other embodiments, the one or more exosomes in the plurality of exosomes have a diameter of about 40 nm to 100 nm and are at least about 3 kDa. In other embodiments, the plurality of exosomes is derived from stem cells, progenitors, and/or precursor cells. In other embodiments, the stem cells, progenitors, and/or precursor cells include cardiosphere-derived cells (CDCs). In other embodiments, the stem cells, progenitors, and/or precursor cells include endothelial precursor cells (EPCs) and/or mesenchymal stem cells (MSCs). In other embodiments, the plurality of exosomes includes a protein. In other embodiments, the plurality of exosomes includes a lipid. In other embodiments, the cell type is a fibroblast. In other embodiments, the one or more properties includes protein expression and/or surface marker expression. In other embodiments, the one or more properties include one or more RNA transcript expression levels. Further described herein is a quantity of converted cells made by the aforementioned method. In various embodiments, altering a cell may include altering Mφ cells, which may include enhancing expression of one or more of Arg1, Il10, and Pparg, elevated Arg1/Nos2 ratio, optionally including Lyve-1, and p50 expression, In other embodiments, altering Mφ may include enhancing expression of one or more of CD68, CD80, CD86, CD11b, CD45, and FSC. In various embodiments, the Mφ are from cardiac, peritoneal, spleen and/or bone marrow-derived sources.

Further described herein is an in vivo method of altering a cell, including selecting a subject, and administering a composition including a plurality of exosomes to the subject, wherein adhesion between one or more exosomes in the plurality of exosomes and a starting cell type is capable of altering one or more properties of the starting cell type, and generating a converted cell type. In other embodiments, the composition includes a plurality of exosomes from stem cells, progenitors, and/or precursor cells grown in serum-free media, wherein the plurality of exosomes includes one or more exosomes with a diameter of about 40 nm to 100 nm, further wherein the one or more exosomes include one or more microRNAs including miR-146a, miR22, and miR-24, and are CD63+, CD105+, or both and are at least about 3 kDa. In other embodiments, administering a composition includes 1×10⁸ or more exosomes in a single dose. In other embodiments, the single dose is administered multiple times to the subject. In other embodiments, administering a composition includes one or more of intra-arterial infusion, intravenous infusion, and injection. In other embodiments, injection includes percutaneous injection. In other embodiments, injection includes injection into heart muscle. In other embodiments, administration is at the site of diseased and/or dysfunctional tissue. In other embodiments, administration is not at the site of diseased and/or dysfunctional tissue. In various embodiments, altering a cell in vivo may include altering Mφ cells, which may include enhancing expression of one or more of Arg1, Il10, and Pparg, elevated Arg1/Nos2 ratio, optionally including Lyve-1, and p50 expression. In other embodiments, altering Mφ may include enhancing expression of one or more of CD68, CD80, CD86, CD11b, CD45, and FSC. In various embodiments, the Mφ are from cardiac, peritoneal, spleen and/or bone marrow-derived sources.

Also described herein is a composition of cells made by a method, including providing a plurality of exosomes, adding to a starting cell type, the plurality of exosomes, wherein the plurality of exosomes includes one or more exosomes with a diameter of about 40 nm to 100 nm, further wherein the one or more exosomes include one or more microRNAs including miR-146a, miR22, and miR-24, and are CD63+, CD105+, or both and are at least about 3 kDa, wherein adhesion between one or more exosomes in the plurality of exosomes and the starting cell type is capable of altering one or more properties of the starting cell type, and generating a composition of a converted cell type. In other embodiments, the one or more properties includes one or more RNA transcript expression levels. In other embodiments, the one or more RNA transcript expression levels include RNA transcript cognate to one or more microRNAs selected from the group consisting of: miR-146a, miR22, and miR-24.

Described herein is a method of administering a plurality of exosomes including selecting a subject and administering a composition including a plurality of exosomes to the subject, wherein administration consists of one or more of: intra-arterial infusion, intravenous infusion, and injection. In other embodiments, injection includes percutaneous injection. In other embodiments, injection includes injection into heart muscle.

In certain embodiments, administering a composition includes 1×10⁸ or more exosomes in a single dose. In other embodiments, administering a composition includes a dosage of 1×10⁸, 1×10⁸ to 1×10⁹, 1×10⁹ to 1×10¹⁰, 1×10¹⁰ to 1×10¹¹, 1×10¹¹ to 1×10¹², 1×10¹² or more exosomes. In other embodiments, the numbers of exosomes is relative to the number of cells used in a clinically relevant dose for a cell-therapy method. For example, it has been demonstrated that 3 mL/3×10⁵ CDCs, is capable of providing therapeutic benefit in intracoronary administration, and therefore, a plurality of exosomes as derived from that number of cells in a clinically relevant dose for a cell-therapy method. In various embodiments, administration can be in repeated doses. In other embodiments, administering a composition includes about 1×10⁵ to about 1×10⁸ or more CDCs in a single dose. In another example, the number of administered CDCs includes intracoronary 25 million CDCs per coronary artery (i.e., 75 million CDCs total) as another baseline for exosome dosage quantity. In various embodiments, exosome quantity may be defined by protein quantity, such as dosages including 1-10, 10-25, 25-50, 50-75, 75-100, or 100 or more mg exosome protein. In various embodiments, administration can be in repeated doses. For example, defining an effective dose range, dosing regimen and route of administration, may be guided by studies using fluorescently labeled exosomes, and measuring target tissue retention, which can be >10×, >50×, or >100× background, as measured 5, 10, 15, 30, or 30 or more min as a screening criterion. In certain embodiments, >100× background measured at 30 mins is a baseline measurement for a low and high dose that is then assess for safety and bioactivity (e.g., using MRI endpoints: scar size, global and regional function).

In certain embodiments, a single dose is administered multiple times to the subject. In certain embodiments, the multiple administrations to the subject includes of two or more of intra-arterial infusion, intravenous infusion, and injection. In other embodiments, injection includes percutaneous injection. In other embodiments, injection includes injection into heart muscle.

In certain embodiments, the plurality of exosomes from stem cells, progenitors, and/or precursor cells are grown in serum-free media, wherein the plurality of exosomes includes one or more exosomes with a diameter of about 40 nm to 100 nm and at least about 3 kDa. In certain embodiments, the stem cells, progenitors, and/or precursor cells include cardiosphere-derived cells (CDCs). In certain embodiments, the CDCs are confluent when isolating the plurality of exosomes. In certain embodiments, the plurality of exosomes includes one or more exosomes including one or more microRNAs selected from the group consisting of: miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In certain embodiments, the one or more microRNAs include miR-146a, miR22, and miR-24. In certain embodiments, the plurality of exosomes includes one or more exosomes that are CD63+, CD105+, or both. In certain embodiments, the stem cells, progenitors, and/or precursor cells include endothelial precursor cells (EPCs) and/or mesenchymal stem cells (MSCs). In certain embodiments, the subject has a heart related disease and/or condition. In certain embodiments, the heart related disease and/or condition includes myocardial infarct. In certain embodiments, the heart related disease and/or condition includes heart failure. In certain embodiments, the heart failure is associated with Duchenne muscular dystrophy. In certain embodiments, administration is at the site of diseased and/or dysfunctional tissue. In certain embodiments, administration is not at the site of diseased and/or dysfunctional tissue.

Further described herein is a method of improving cardiac performance in a subject including, selecting a subject, administering a composition including a plurality of exosomes to the individual, wherein administration of the composition improves cardiac performance in the subject. In some embodiments, this includes a decrease in right ventricle systolic pressure. In other embodiments, there is a reduction in arteriolar narrowing, or pulmonary vascular resistance. In other embodiments, improving cardiac performance can be demonstrated, by for example, improvements in baseline ejection volume. In other embodiments, improving cardiac performance relates to increases in viable tissue, reduction in scar mass, improvements in wall thickness, regenerative remodeling of injury sites, enhanced antiogenesis, improvements in cardiomyogenic effects, reduction in apoptosis, and/or decrease in levels of pro-inflammatory cytokines.

In certain embodiments, the method of improving cardiac performance includes, selecting a subject in need of treatment for a heart related disease and/or condition, administering a composition including a plurality of exosomes to the individual, wherein administration of the composition treat the subject. In various embodiments, the heart related disease and/or condition includes heart failure. In various embodiments, the plurality of exosomes range in size from 30 to 300 nm. In various embodiments, the plurality of exosomes range in size from 40 to 100 nm. In certain embodiments, the plurality of exosomes is cardiosphere-derived cell (CDC) exosomes. In certain embodiments, the plurality of exosomes includes one or more exosomes that are CD63+, CD105+, or both. In various embodiments, the exosomes include microRNAs miR-146a, miR148a, miR22, miR-24, miR-210, miR-150, miR-140, miR-19a, miR-27b, miR-19b, miR-27a, miR-376c, miR-128, miR-320a, miR-143, miR-21, miR-130a, miR-9, miR-185, miR-23a, miR-302b, miR-181b, miR-155, miR-200, miR-7, miR-423, let-7b, let-7f, miR-21, let-7e, and mir-23b. In other embodiments, the exosomes are 2-5 kDa, such as 3 kDa. In other embodiments, administering a composition includes a dosage of 1×10⁸, 1×10⁸ to 1×10⁹, 1×10⁹ to 1×10¹⁰, 1×10¹⁰ to 1×10¹¹, 1×10¹¹ to 1×10¹², 1×10¹² or more exosomes. In other embodiments, the numbers of exosomes is relative to the number of cells used in a clinically relevant dose for a cell-therapy method. For example, it has been demonstrated that 3 mL/3×10⁵ CDCs, is capable of providing therapeutic benefit in intracoronary administration, and therefore, a plurality of exosomes as derived from that number of cells in a clinically relevant dose for a cell-therapy method. In various embodiments, administration can be in repeated doses. In other embodiments, administering a composition includes about 1×10⁵ to about 1×10⁸ or more CDCs in a single dose. In another example, the number of administered CDCs includes intracoronary 25 million CDCs per coronary artery (i.e., 75 million CDCs total) as another baseline for exosome dosage quantity. In various embodiments, exosome quantity may be defined by protein quantity, such as dosages including 1-10, 10-25, 25-50, 50-75, 75-100, or 100 or more mg exosome protein. In various embodiments, administering a composition includes multiple dosages of the exosomes. In various embodiments, the repeated or sequentially-applied doses are provided for treatment of an acute disease and/or condition. In various embodiments, the repeated or sequentially-applied doses are provided for treatment of a chronic disease and/or condition. In other embodiments, administering a composition includes percutaneous injection. In other embodiments, administering a composition includes injection into heart muscle. In other embodiments, administering a composition includes myocardial infusion. In other embodiments, administering a composition includes use of a intracoronary catheter. In other embodiments, administration a composition includes intra-arterial or intravenous delivery. Additional delivery sites include any one or more compartments of the heart, such as myocardium, associated arterial, venous, and/or ventricular locations. In certain embodiments, administration can include delivery to a tissue or organ site that is the same as the site of diseased and/or dysfunctional tissue. In certain embodiments, administration can include delivery to a tissue or organ site that is different from the site or diseased and/or dysfunctional tissue. In certain embodiments, the delivery is via inhalation or oral administration. In various embodiments, administration of exosomes can include combinations of multiple delivery techniques, such as intravenous, intracoronary, and intramyocardial delivery. In other embodiments, exosome therapy is provided in combination with standard therapy for a disease and/or condition. This may include co-administration of the exosomes with a therapeutic agent.

### Example 1

### CDCs as a Source of Exosomes

As described, a critical scientific and medical question is understanding whether stem cells might be helpful in not only preventing or ameliorating disease and/or conditions, but actually capable of treating heart disease and related conditions via regeneration and repair of damaged cells and promotion of vascular cell growth. It is suggested that therapeutic effects of stem cells via regeneration can be significantly enhanced by directly delivering exosomes produced by such stem cells as an alternative to delivering the cell themselves. Preliminary studies by the Inventors have shown that in a variety of scenarios, CDC-derived exosomes are indeed capable of delivering therapeutic benefits. This includes, for example, intracoronary delivery in ischemia/reperfusion (IR) injury, percutaneous injection in a myocardial infarct mode, intravenous infusion of CDC exosomes for pulmonary arterial hypertension (PAH) is capable of noticeable benefits, as shown via echocardiography.

Such cardiosphere derived cells (CSCs) are obtained via endomyocardial biopsies from the right ventricular aspect of the interventricular septum as obtained from healthy hearts of deceased tissue donors. Cardiosphere-derived cells are derived as described previously. See Makkar et al., (2012). “Intracoronary cardiosphere-derived cells for heart regeneration after myocardial infarction (CADUCEUS): a prospective, randomized phase 1 trial.” Lancet 379, 895-904 (2012), which is fully incorporated by reference herein.

In brief, heart biopsies are minced into small fragments and briefly digested with collagenase. Explants were then cultured on 20 mg/ml fibronectin-coated dishes. Stromal-like flat cells and phase-bright round cells grow out spontaneously from tissue fragments and reach confluence by 2-3 weeks. These cells are harvested using 0.25% trypsin and cultured in suspension on 20 mg/ml poly d-lysine to form self-aggregating cardiospheres. cardiosphere-derived cells (CDCs) are obtained by seeding cardiospheres onto fibronectin-coated dishes and passaged. All cultures are maintained at 5% CO2 at 37° C., using IMDM basic medium supplemented with 20% FBS, 1% penicillin/streptomycin, and 0.1 ml 2-mercaptoethanol. See Makkar et al., (2012). “Intracoronary cardiosphere-derived cells for heart regeneration after myocardial infarction (CADUCEUS): a prospective, randomised phase 1 trial.” Lancet 379, 895-904 (2012), which is fully incorporated by reference herein.

### Example 2

### Media Conditioning and Exosome Purification

Exosomes are harvested from CDCs at passage 4. One can also isolate exosomes from normal human dermal fibroblasts (NHDF), cells that have been previously utilized as controls providing no salutary benefit, as a control. CDCs and NHDFs re conditioned in serum-free media for 15 days at 100% confluence. Aspirated media is then centrifuged at 3,000×g for 15 min to remove cellular debris. Exosomes were then isolated using Exoquick Exosome Precipitation Solution (FIG. 2).

Exosome pellets are resuspended in the appropriate media and used for assays. Expression of the conserved exosome marker CD63 is verified using ELISA. RNA content of exosome pellets can also be quantified using a Nanodrop spectrophotometer. Exosomal RNA degradation is performed by suspending exosome pellets in 2 ml of PBS. To one sample, 100 ml of Triton X-100 (Sigma Aldrich) is added to achieve 5% triton concentration. Exosomes are treated with 0.4 mg/ml RNase A treatment for 10 min at 37° C. Samples are further treated with 0.1 mg/ml Proteinase K for 20 min at 37° C. RNA is purified from samples using an microRNA isolation kit. RNA levels are measured using Nanodrop.

### Example 3

### Mass Spectrometry Analysis on Exosome Pellets

Proteins were prepared for digestion using the filter-assisted sample preparation (FASP) method. Concentrations were measured using a Qubitfluorometer (Invitrogen). Trypsin was added at a 1:40 enzyme-to-substrate ratio and the sample incubated overnight on a heat block at 37° C. The device was centrifuged and the filtrate collected. Digested peptides were desalted using C18 stop-and-go extraction (STAGE) tips. Peptides were fractionated by strong anion exchange STAGE tip chromatography. Peptides were eluted from the C18 STAGE tip and dried. Each fraction was analyzed with liquid chromatography-tandem mass spectrometry. Samples were loaded to a 2 cm 3 100 mm I.D. trap column. The analytical column was 13 cm 3 75 mm I.D. fused silica with a pulled tip emitter. The mass spectrometer was programmed to acquire, by data-dependent acquisition, tandem mass spectra from the top 15 ions in the full scan from 400 to 1,400 m/z. Mass spectrometer RAW data files were converted to MGF format using msconvert. MGF files were searched using X!Hunter against the latest spectral library available on the GPM at the time. MGF files were also searched using X!!Tandem using both the native and k-score scoring algorithms and by OMSSA. Proteins were required to have one or more unique peptides with peptide E-value scores of 0.01 or less from X!!Tandem, 0.01 or less from OMSSA, 0.001 or less and theta values of 0.5 or greater from X!Hunter searches, and protein E-value scores of 0.0001 or less from X!!Tandem and X!Hunter. Myocyte Isolation Neonatal rat cardiomyocytes (NRCMs) were isolated from 1- to 2-day-old Sprague Dawley rat pups and cultured in monolayers as described.

### Example 4

### CDC Exosomes are Enriched in MicroRNAs Reported as Providing Therapeutic Effects

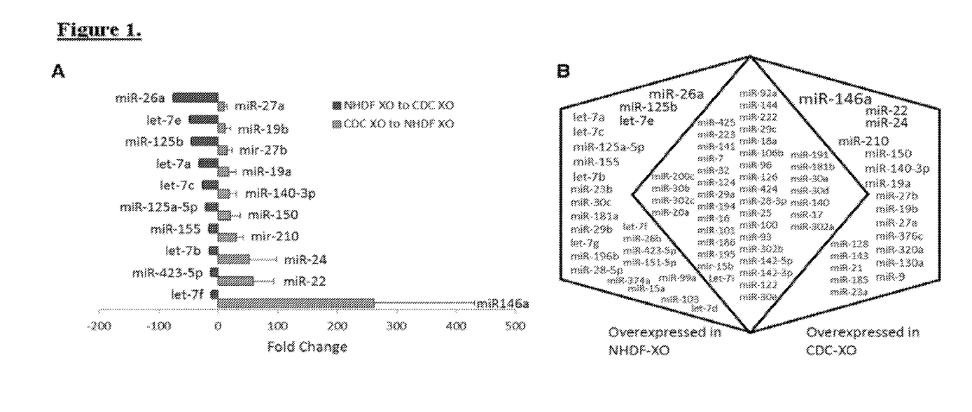

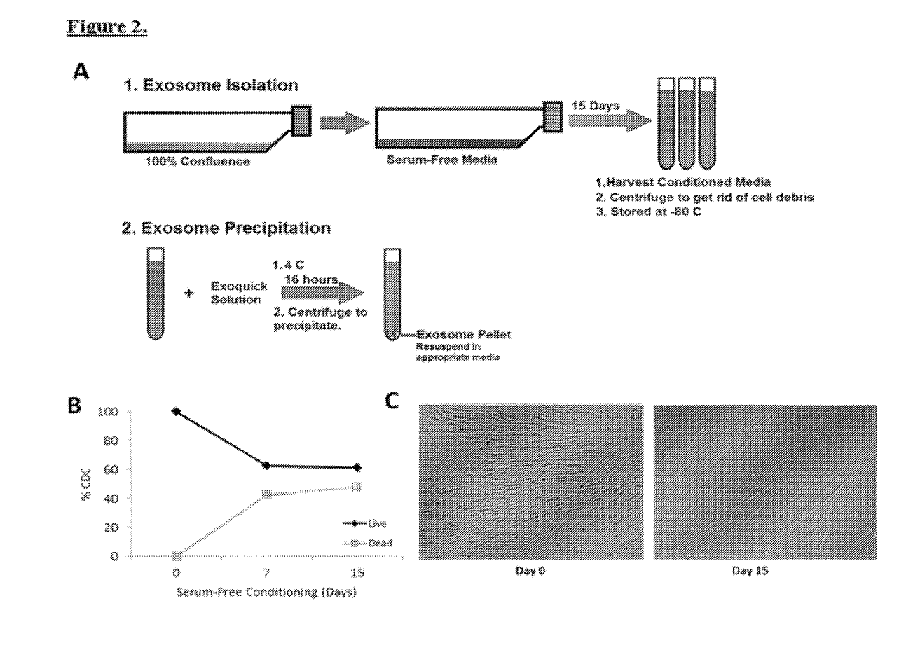

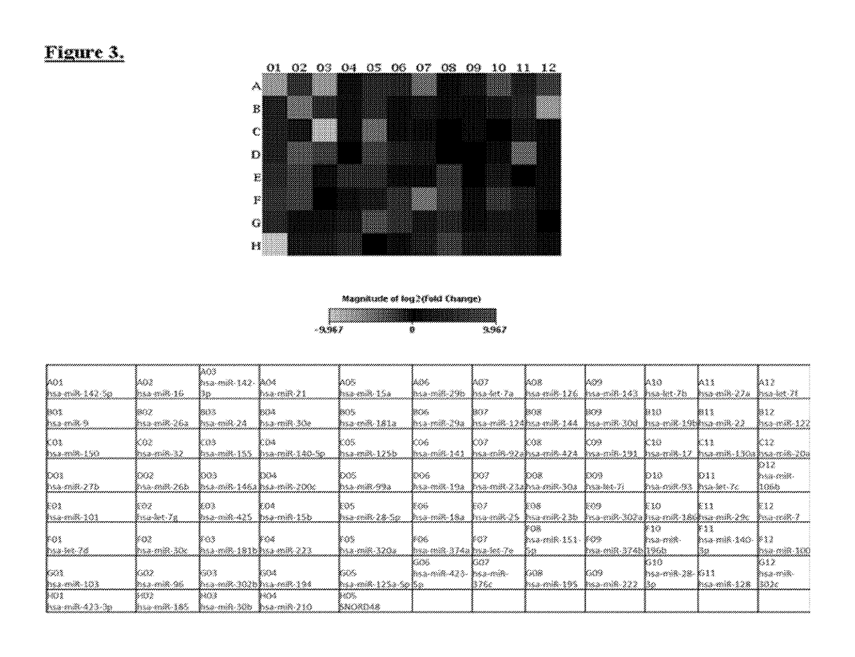

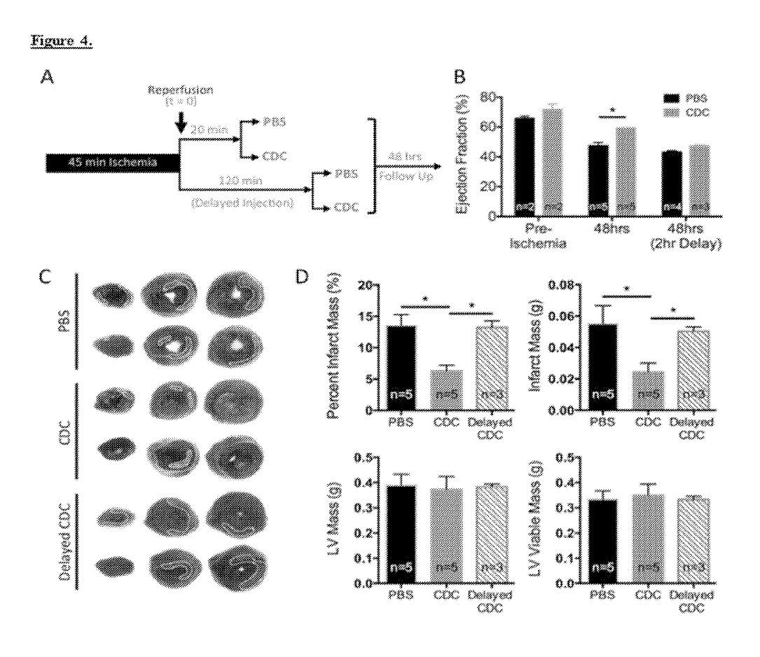

To investigate the basis of the therapeutic benefit of CDC exosomes, the Inventors compared their microRNA repertoire to that of NHDF exosomes using a PCR microarray of the 88 best-defined microRNAs. The microRNA content of the two cell types differed dramatically. Forty-three microRNAs were differentially present in the two groups; among these, miR-146a was the most highly enriched in CDC exosomes (262-fold higher than in NHDF exosomes; FIGS. 1A, 1B, and 3).

Recently, the therapeutic effects of microRNAs such as miR-146a, as derived from CDCs, have been shown as mediate some of the therapeutic benefits of CDC exosomes. For example, miR-146a leads to thicker infarct wall thickness and increased viable tissue in a mouse model of myocardial infarct. Ibrahim, et al., “Exosomes as critical agents of cardiac regeneration triggered by cell therapy.” Stem Cell Reports. 2014 May 8; 2(5):606-19, which is fully incorporated by reference herein.

### Example 5

### Dosage Studies

To examine safety and efficacy of CDC-derived exosomes, the Inventors performed a dose finding study in Wistar-Kyote rats (WKY, aged 8-12 weeks). Briefly, conditioned media was collected from human CDCs in serum-free media for 4 days when exosomes were precipitated using ExoQuick-TC®.

As a starting dose, CDC-derived exosomes were isolated from a equivalent, and previously-validated CDC dose for intracoronary delivery following ischemia/reperfusion (IR). That is, 3 mL/3×10⁵ CDCs, as previously described. CDC-derived exosome protein quantity was determined (˜700 μg/10 mL) and doses were titrated. For in vivo analyses, WKY rats underwent 45 minutes of ischemia followed by 20 minutes of reperfusion.

Animals were then randomly allocated to receiver either PBS or a titrated dose of CDC-derived exosomes (derived from 10 mL, 3 mL, or 1 mL CDC-conditioned media). Two days following injury, all CDC-derived exosomes doses (10 mL, 3 mL, and 1 mL) conferred a significant reduction in percent infarct mass relative to PBS control (PBS 13.56% v. 10 mL 6.36%; 3 mL 6.94%; 1 mL 8.03%; p<0.05). Similarly, ejection fraction was preserved in the lower doses (PBS 43.07% v. 10 mL 57.40%; 3 mL 58.17%; 1 mL 55.49%; p<0.05). Interestingly, these EXOCDC express a unique surface protein signature that includes some generic markers from exosomes (CD63, HSP70, but no CD9 or CD81), as well as CDC-specific markers (CD105).

These results demonstrate that CDC-derived exosomes, delivered via the intracoronary route 20 mins post-IR, are cardioprotective. The evaluation of efficacy at 48 hours rules out regenerative effects, which manifest themselves over weeks. The ability to delay administration after IR is highly suggestive of clinically relevant effects, as CDC-derived exosomes may be useful cardioprotective therapeutic candidates adjunctive to routine therapy for myocardial infarction.

### Example 6

### Intracoronary Infusion of Exosome Therapeutic

An example of the described technique, exosomes are isolated from human CDCs as described using a technique such as ExoQuick® precipitation in order to generate a composition include a population of exosomes ranging in size from 30-100 nm that are enriched in biological agents capable of cardiac repair (e.g., proteins, surface antigens such as CD105, microRNAs such as miR-146a). A single dose, such as 3 mL/3×10⁵ CDCs, can be delivered to a subject in need of treatment for a heart related diseases and/or conditions, which can include both acute and chronic diseases and/or conditions. Importantly, the above results indicate that exosomes provide both cardioprotective and regenerative effects, thereby providing multiple timepoints for administration ranging from immediately after an acute event (e.g., myocardial infarct) or at much later timepoints such as weeks and/or months during the progression of chronic disease (e.g., congestive heart disease).

Such administration may occur as a single dose or a series of repeated doses, and it understood that dosages may be provided by variable routes of administration combined together. Administration may be via intracoronary infusion as delivered through the central lumen of a balloon catheter positioned in the coronary artery, such as via over-the-wire balloon catheter, with a subtended by a patent coronary artery. Subsequent repeat doses can also be via intracoronary infusion, but may rely on other methods of administration (e.g., intravenous infusion).

A variety of techniques may be relied upon to evaluate the therapeutic effects of exosome therapy. This includes echocardiographic assessment, wherein wall thickness, ejection volume or a variety of other parameters may indicate cardiac improvement. Other examples include hemodynamic measurement.

### Example 7

### CDCs Modulate Macrophage Inflammatory Response

Wistar-Kyoto rats (age 8-12 weeks) underwent 45 mins of ischemia followed by 20 mins of reperfusion, then intracoronary (i.c.) infusion of either saline or CDCs (5×10⁵). The use of a 48 hour endpoint allowed the selective study of cardioprotection. CDC-treated animals had preserved ejection fraction (59.2% v. 47.4%, p<0.001) and reduced infarct size (TTC: 6.3% v. 13.6%, p<0.01). The finding that CDC-treated hearts contained fewer CD68+ Mφ (p<0.05) suggested a mechanistic role for Mφ. When isolated from CDC-treated heart, Mφ secreted lower amounts of proinflammatory cytokines (Nos2, Tnf, IL1b, p<0.05). Systemic depletion of Mφ with clodronate liposomes attenuated the benefits of CDC therapy post-MI (p<0.05).

### Example 8

### Polarization of Mφ

In vitro, Mφ conditioned by transwell exposure to CDCs (MCDC) exhibited distinct gene profiles relatively to proinflammatory M1 or healing M2 polarization states (M1: NOS2, M2: Arg, Pparg, MCDC: IL10). Adoptive transfer of selective Mφ populations into the heart (i.c: 20 min post-reflow) revealed that MCDCs, but not M1 or M2 Mφ could recapitulate the reduction in infarct size (MCDC 4.5%, M1: 14.0%, M2 10.8%, p<0.05). In vitro co-culture shows that MCDC selectively reduced cardiomyocyte apoptosis following oxidant stress (MCDC 9.9%, M1 39.4%, M2 37.4%, p<0.01). These results confirm that CDCs are cardioprotective when administered 20 mins after reflow. The shorter timeframe distinguishes this form of cardioprotection from precondition or ischemic post-conditioning. Various lines of evidence indicate that CDCs work by polarizing Mφ toward a cardioprotective phenotype. Such results suggest adjunctive use of CDCs post-MI to limit infarct size.

### Example 9

### MiR-146a Effect on Immune Infiltration

Attenuating the inflammatory immune response is not necessarily abrogating it altogether. Innate immune cells including macrophages have been shown to play pro regenerative roles. The above results indicate that macrophage trafficking is not affected by CDC treatment, but rather, macrophages treated with CDCs switch away from an M1 (proinflammatory) toward an M2-like anti-inflammatory and pro-healing phenotype.

### Example 10

### Study Design

As described, cardiosphere-derived cells (CDCs) confer both cardioprotection and regeneration in acute myocardial infarction (MI). While the regenerative effects of CDCs in chronic settings have been studied extensively, little is known about how CDCs confer cardioprotection.

To investigate the underlying mechanisms of CDC-mediated cardioprotection, the Inventors established an in vivo rat model of MI induced by ischemia-reperfusion (IR) injury and in vitro co-culture assays to establish how CDCs protect stressed cardiomyocytes. In addition to observing cardiomyocyte apoptosis and potential oxidative stress protection of ventricular myocytes, the Inventors attempted to identify mechanisms by which CDCs possibly modify myocardial leukocyte populations after ischemic injury. Finally, both in vitro co-culture assays and an in vivo adoptive transfer rat model post-IR, were developed to establish whether CDC-conditioned Mφ are capable of conferring cardioprotection by attenuating cardiomyocyte apoptosis and protecting from oxidative stress.

### Example 11

### Experimental Protocol, Animals, & Surgical Procedures

For animal studies, 7-10 week old female Wistar-Kyoto (WKY) rats (Charles River Labs, Wilmington, Mass.) were utilized for all in vivo experimental protocols. To induce ischemia-reperfusion (IR) injury, rats were provided general anesthesia and then a thoracotomy was performed at the 4^{th} intercostal space to expose the heart and left anterior descending (LAD) coronary artery. A 7-0 silk suture was then used to ligate the LAD, which was subsequently removed after 45 minutes to allow for reperfusion. Twenty minutes (or 2 hours) later, cells (or PBS control) were injected into the left ventricular cavity with an aortic crossclamp, over a period of 20 seconds. To induce myocardial infarction (MI), the LAD was permanently ligated and cells (or PBS control) were injected into 4 regions within ischemic border zone.

For the Inventors' Mφ depletion studies, WKY rats were intravenously injected with 1 mL (5 mg/mL) clodronate (Cl₂MDP: dichloromethylene diphosphonate) liposomes (Clodrosome, Encapsula NanoSciences) one day prior to, and one day following, IR injury.

### Example 12

### Rat Cardiosphere-Derived Cell (CDC) Isolation

Allogeneic CDCs were derived as previously described. Briefly, heart tissue from Sprague-Dawley (SD) rats (Charles River Labs, Wilmington, Mass.) was isolated, minced, enzymatically digested, then plated to allow cardiac explant cell growth. After 7-10 days, cells were harvested and plated into a non-adherent cell culture dish to support cardiosphere formation. After 2 days, cardiospheres were isolated then plated on an adherent dish to allow CDC growth. Cells were subsequently expanded to passage 4-6 and utilized for all experimental work. Based on previously established in vivo dosing studies, the Inventors utilized 5×10⁵ CDCs resuspended in 100 μL PBS (5% Heparin, 1% Nitroglycerin) for treatment post-IR and 2×10⁶ CDCs resuspended in 120 μL PBS post-MI.

### Example 13

### Macrophage Cell Isolation and Differentiation from Origin Sites

Cardiac.

WKY rats underwent MI and then were randomly allocated to receive either PBS or CDCs, as described above. After 48 hours, hearts were harvested following perfusion with PBS. The infarct and infarct border zones were isolated, minced, enzymatically digested (Liberase enzyme, Roche), and then filtered through a 70 μm mesh. Mononuclear cells were isolated using a density gradient (Histopaque 1083, Sigma-Aldrich), washed, resuspended in RPMI (supplemented with 1% FBS), and then plated. Following a two hour incubation at 37° C., 5% CO₂, the attached cardiac Mφ (cMφ) cells were washed with PBS and then incubated with RPMI for downstream analyses.

Peritoneal.

Brewer's Thioglycollate solution (3% in PBS; Sigma-Aldrich) was injected into the peritoneal cavity of WKY rats. Three days later, Mφ cells were harvested following intraperitoneal lavage with PBS. Cells were filtered through a 70 μm mesh, lysed with ACK buffer (Invitrogen), then resuspended and plated using RPMI. Following a 2 hour incubation at 37° C., 5% CO₂, the attached peritoneal Mφ (pMφ) cells were washed with PBS and then incubated with RPMI for further analyses.

Bone Marrow (BM)-Derived Mφ.

Femurs were isolated from 7-10 week old WKY rats. BM were isolated, flushed with PBS (containing 1% FBS, 2 mM EDTA; FACS Buffer), and filtered through a 70 μm mesh. Red blood cells were lysed with ACK buffer (Invitrogen), and resuspended in IMDM (Gibco) containing 10 ng/mL M-CSF (eBioscience) for plating. After 3 days the media was exchanged. On day 7-8 BMDMs were incubated overnight (˜18 hours) to polarize toward M₁ (100 ng/mL LPS and 50 ng/mL IFNγ; Sigma-Aldrich and R&D Systems, respectively), M₂ (10 ng/mL IL-4 and IL13; R&D Systems), or M_{CDC} (CDC transwell co-culture). For in vivo infusion, 1×10⁶ BMDMs were labeled with DiI (Vybrant Cell-Labeling Solutions, Invitrogen) according to the manufacturer's protocol then infused following IR, as described above.

### Example 14

### Total Leukocyte Isolation

WKY rats underwent IR and then were randomly allocated to receive either PBS or CDC, as described above. After 48 hours, blood was collected from the right atrium in heparinized tubes and hearts were collected following perfusion with PBS.

Peripheral Blood.

Blood was separated by centrifugation at 1850×g for 15 minutes. The buffy coat layer was isolated and resuspended in FACS buffer. Following centrifugation, red blood cells were lysed from the pellet using ACK buffer (Invitrogen). The resulting white blood cells were used for flow cytometric analyses.

Cardiac.

The infarct and infarct border zones were isolated, minced, digested with Liberase enzyme TM (Roche), and then filtered through a 70 μm mesh. The resulting cell suspension was used for flow cytometric analyses.

### Example 15

### Physiological, Molecular and Immuno-Characterization

All antibodies used for this study are included in Table 1. Analyses were performed using a CyAn ADP (Beckman Coulter) flow cytometer. Freshly-isolated samples were resuspended in FACS buffer (PBS containing 1% FBS and 2 mM EDTA) and stained with conjugated antibody for 20-30 minutes at 4° C. Cells were washed and resuspended with FACS buffer for flow cytometric analyses where inflammatory cell populations were designated following gating/stratification of their marker profile.

Cardiac Functional Measurements.

Transthoracic echocardiography (Vevo 770, Visual Sonics, Toronto, ON) was performed prior to, and following, IR injury at the designated time points (pre-ischemia, 48 hours, 2 weeks). Two-dimensional short- and long-axes were visualized. Three representative cycles were captured for each animal/time point and measurements for left-ventricular end-systolic dimension (LVESD), left-ventricular end-diastolic dimension (LVEDD), and ejection fraction (EF) were obtained and averaged.

Tissue Harvest and Cryostat Sectioning.

Hearts were arrested in diastole following intraventricular injection of 10% potassium chloride (KCl) then excised and washed in PBS. The atria and base above the infarct were removed. The tissue was fixed in 4% PFA (4% paraformaldehyde in PBS), processed through a sequential sucrose gradient (10%, 20%, 30% in PBS), embedded in OCT compound (Tissue-Tek OCT, Torrance, Calif.), and then kept at −80° C. until sectioning. Tissue samples were cut at 5 um thickness.

Histology, Immunohistochemistry, and Immunocytochemistry and 2,3,5-Triphenyl-2H-Tetrazolium Chloride (TTC).

Two days following IR injury, hearts were arrested in diastole following intraventricular injection of 10% KCl. Hearts were then excised, washed in PBS, and cut into serial sections of ˜1 mm in thickness (from apex to basal edge of infarction). Sections were incubated with TTC (1% solution in PBS) for 20 minutes in the dark, washed with PBS, then imaged and weighed. Infarcts were delineated from viable tissue (white versus red, respectively) and analyzed using ImageJ software. Infarct mass, viable mass, and LV mass were calculated by extrapolating for infarct and non-infarct volumes (based on the areas calculated from both sides of a tissue section) and weight of the tissue. Percentage infarct mass was calculated using (Infarct Mass/Viable Mass)×100%.

Masson's Trichrome.

OCT-cut tissue were stained according to the manufacturer's protocol (Sigma-Aldrich), then mounted and imaged. Morphometric analyses of the infarcted tissue were performed using ImageJ software. Infarct thickness and size measurements were obtained from the mid-papillary level of the infarcted heart.

Immunohistochemistry.

For analyses of cardiomyocyte size and inflammatory cell distribution, OCT-embedded tissue sections were fixed with 4% PFA and stained with the following primary antibodies for confocal microscopy: mouse anti-rat α-actinin (Sigma), mouse anti-rat CD68 (AbD Serotec), mouse anti-rat CD45 (BD Pharmingen). The appropriate fluorescently-conjugated secondary antibodies (Invitrogen) were applied prior to mounting using Fluoroshield with DAPI (Sigma). To detect apoptotic cardiomyocytes, the Inventors performed a TdT dUDP Nick-End Labeling assay (TUNEL, Roche) according to the manufacturer's protocol and stained with α-actinin and DAPI. To determine cardiomyocyte size, the Inventors utilized an Alexa Fluor 488-conjugated wheat-germ agglutinin (WGA, Invitrogen Life Technologies) stain in conjunction with α-actinin and DAPI. Only cardiomyocytes with centrally-located nuclei were utilized for cell size determination.

Immunocytochemistry.

Peritoneal and cardiac macrophage cells were cultured on fibronectin coated slides, fixed with 4% PFA, and stained with mouse anti-rat CD68 (AbD Serotec). The appropriate fluorescently-conjugated antibody was added and then cells were counterstained with DAPI. For live, cultured BMDM cells, Hoechst 33342 (Sigma 14533) was utilized to distinguish nucleated/multinucleated cells.

TABLE 1
Antibodies used for flow cytometry.

| Antibody | Fluorophore | Clone | Supplier |
|---|---|---|---|
| CD45 | FITC | OX-1 | BD Biosciences |
| CD45 | PE-Cy7 | OX-1 | BD Biosciences |
| CD11b | APC | WT.5 | BD Biosciences |
| CD11c | FITC | 8A2 | AbD Serotec |
| CD3 | APC | 1F4 | BD Biosciences |
| CD4 | FITC | OX-35 | BD Biosciences |
| CD8a | PE | OX-8 | BD Biosciences |
| CD68 | PE | ED1 | AbD Serotec |
| Granulocyte | FITC | HIS48 | BD Biosciences |
| CD161a | PE | 10/78 | BD Biosciences |
| CD80 | PE | 3H5 | BD Biosciences |
| CD86 | FITC | 24F | BD Biosciences |

### Example 16

### Protein and RNA Isolation and Analysis

Protein.

At the appropriate time point following surgery, the heart was harvested and rinsed in PBS. The border, infarct, and normal zones were dissected, placed in Allprotect tissue reagent (QIAGEN), and stored at −80° C. until use. Tissues were minced, suspended in T-PER (with HALT protease and phosphatase inhibitors, Thermo Scientific) and homogenized with a bead ruptor. For cell culture experiments, cells were lysed with RIPA (with HALT protease and phosphatase inhibitors, Thermo Scientific), scraped off culture plates, and sonicated for 3 cycles of 10 second bursts (Active Motif) on ice. The resulting suspensions were centrifuged at 10,000×g for 15 minutes at 4° C. and the protein supernant collected. Protein concentrations were measured using a BCA assay (Thermo Scientific).

RNA.

At the appropriate time points, cells were washed and collected for RNA isolation using an RNeasy Mini Kit (QIAGEN) according to the manufacturer's protocol. RNA concentration and purity were determined using a NanoDrop spectrophotometer (Thermo Scientific).

Quantitative RT-PCR.

To compare the gene expression level between cells at rest, following co-culture, or after stimulation, the Inventors utilized both SYBR green and Taqman technologies (Applied Biosystems, Foster City, Calif.).

SYBR Green.

To assess gene expression, cDNA was synthesized from mRNA using an RT² First Strand synthesis kit (QIAGEN) according to the manufacturer's protocol. The resulting cDNA was standardized across samples and loaded into the pre-designed RT² Profiler PCR array (QIAGEN) plates. Gene expression was then amplified over the course of 40 cycles and analyzed by ddCt.

TaqMan.

To assess gene expression, cDNA was synthesized from mRNA using the High Capacity cDNA Reverse Transcription Kit (Applied Biosystems) according to the manufacturer's protocol. The resulting cDNA was standardized across samples, and then mixed with master mix and designated primer sets (Life Technologies, Invitrogen). The following predesigned TaqMan primer sets were purchased from Life Technologies: Arg1, Tnf, Nos2, Tgfb1, Il1a, Il1b, Il6, Il10, Il4ra, Ccl3, Ccl5, Pparg, NJkb1, Vegfa, Nod2, Tlr9.

Western Blot Analysis and Protein Array.

Protein samples were prepared for gel electrophoresis (NuPAGE 4-12% Bis-Tris, Invitrogen) according to the manufacturer's protocol. For all experiments, a normalized final loading concentration between 10-30 μg/well was used prior to separation. Proteins were then transferred to a polyvinylidene fluoride (PVDF) membrane (BioRad) for immunoblotting with designated antibodies. Bands were visualized following activation with ECL (Thermo Scientific) and exposure on film (Kodak Carestream Biomax, Sigma).

Rat cytokines were analyzed on a protein array (Raybiotech) according to the manufacturer's protocol. Briefly, tissue lysates were incubated with the antibody array, membranes washed, and then a secondary biotinylated antibody was introduced. Incubation with streptavidin and subsequent exposure with a detection buffer allowed for visualization of dots on film (Kodak Carestream Biomax, Sigma).

### Example 17

### Cytokine Bead Array for Serum Cytokine Analysis

Serum levels of cytokines were analyzed with a FlowCytomix Multiplex bead array (eBioscience) according to the manufacturer's protocol. Briefly, blood was collected from rats after 48 hours post-IR. Serum was then separated by centrifugation and incubated with antibody-coated beads (CCL2, IFNγ, IL-1a, IL-4, TNFα). After the appropriate labeling, beads were resuspended with buffer then analyzed using a CyAn ADP (Beckman Coulter) flow cytometer.

### Example 18

### Neonatal Rat Ventricular Myocyte Isolation and In Vitro Assay

Neonatal rat ventricular myocyte (NRVM) were cultured as previously described. Briefly, hearts were harvested from 2 day old SD rats. Ventricles were isolated, minced, and then enzymatically digested in a solution of Trypsin and Collagenase overnight. Cells were then resuspended in m199 media (10% FBS, glucose, penicillin, vitamin B₁₂, HEPES, and MEM non-essential amino acids (Gibco)) and pre-plated to allow non-cardiomyocyte cells to attach. The resulting NRVM suspension was collected and counted prior to plating for experimental use.

To induce oxidative stress in NRVMs, 10M H₂O₂(Sigma) was diluted in IMDM (Gibco) to a final concentration of 50 μM. Cells were then incubated for 15 minutes at 37° C. prior to media exchange. For in vitro NRVM-Mφ coculture, Mφ were dyed with DiO (Vybrant Cell-Labeling Solutions, Invitrogen) for 3 minutes at 37° C., washed with FACS buffer, resuspended in IMDM, and then added to the NRVM culture dish.

### Example 19

### Allogeneic CDCs Confer Cardioprotection within 20 Minutes of Infusion Post-IR

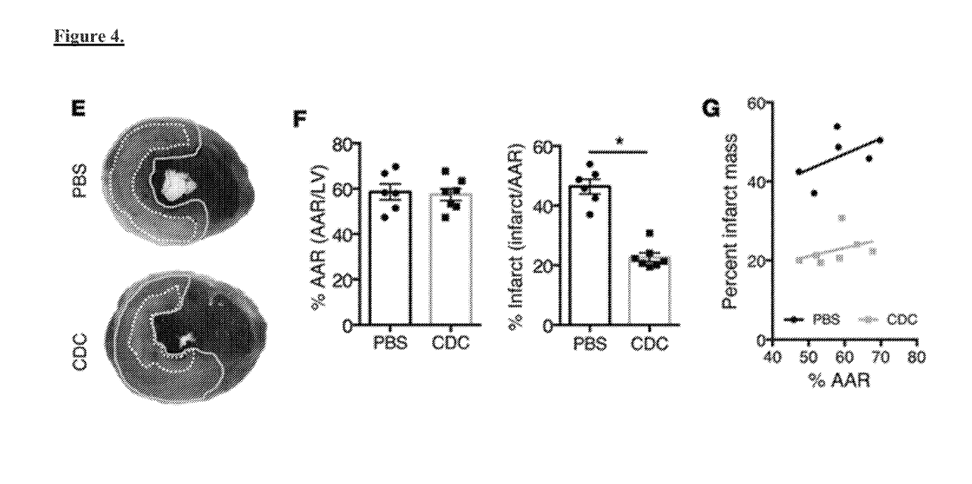

To investigate the role of CDCs in acute cardioprotection, the Inventors designed a protocol that would simulate clinical IR injury. As described, patients with MI undergo prompt angioplasty to reopen the occluded coronary artery. After flow has been re-established, the use of adjunctive therapy can be considered. Adjunctive cell therapy would require thawing of an allogeneic, off-the-shelf product and preparation for administration, which could introduce a delay of up to 20 minutes. Therefore, in the Inventors' rat model the Inventors used 45 minutes of ischemia followed by 20 minutes of reperfusion. Cells were then delivered to the coronary circulation (FIG. 4A). To examine whether cell administration could be delayed further, the Inventors compared the results to those from a ‘delayed infusion’ group in which CDCs were infused 2 hours post-IR (FIG. 4A). In general the Inventors quantified endpoints at 48 hours, to enable study of the cardioprotective effect in isolation, well before the regenerative mechanisms of cardiomyocyte proliferation and activation of endogenous cardioblasts come into play (on a time scale of weeks.

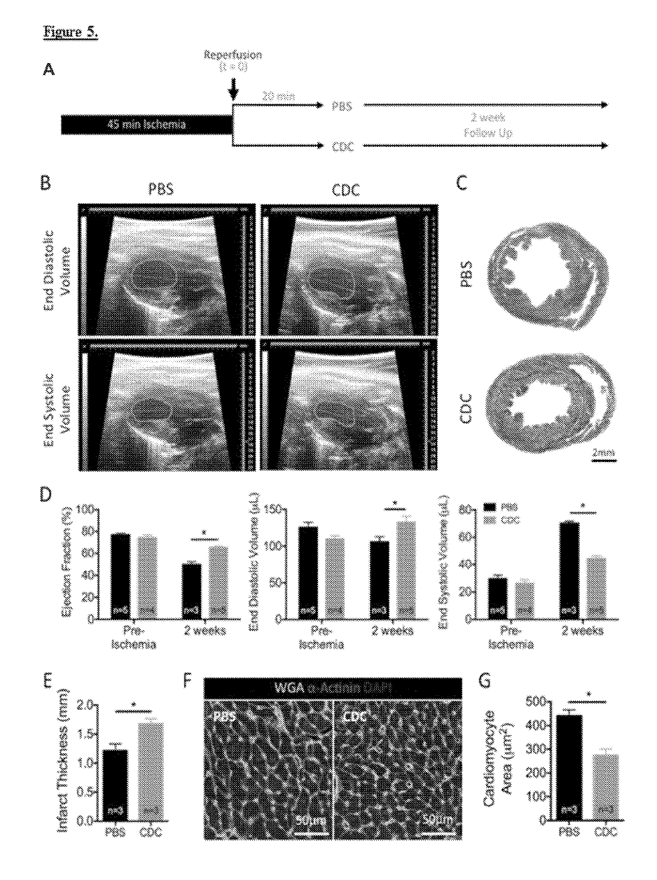

CDC-treated animals exhibited preserved cardiac function (FIG. 4B) and reduced infarct size (FIGS. 4C & 4D), relative to vehicle (PBS) control or delayed infusion rats. While these beneficial effects were observed during the acute reparative phase, the functional and structural benefits of CDC treatment persisted for at least 2 weeks (FIG. 5). During this chronic repair phase, cardiac function did not deteriorate as it did in controls, leading to preservation in LV systolic and diastolic dimensions (FIGS. 5B & 5D), less thinning of the LV anterior wall (FIGS. 5C & 5E) and reduced hypertrophy of surviving cardiomyocytes (FIGS. 5F & 5G). Thus, CDCs acutely-administered post-MI reduce lethal injury at 48 hrs, leading to sustained functional and structural benefits.

### Example 20

### Infusion of CDCs Reduces Cardiac Stress, Attenuating Cardiomyocyte Death and Proinflammatory Cytokine Expression

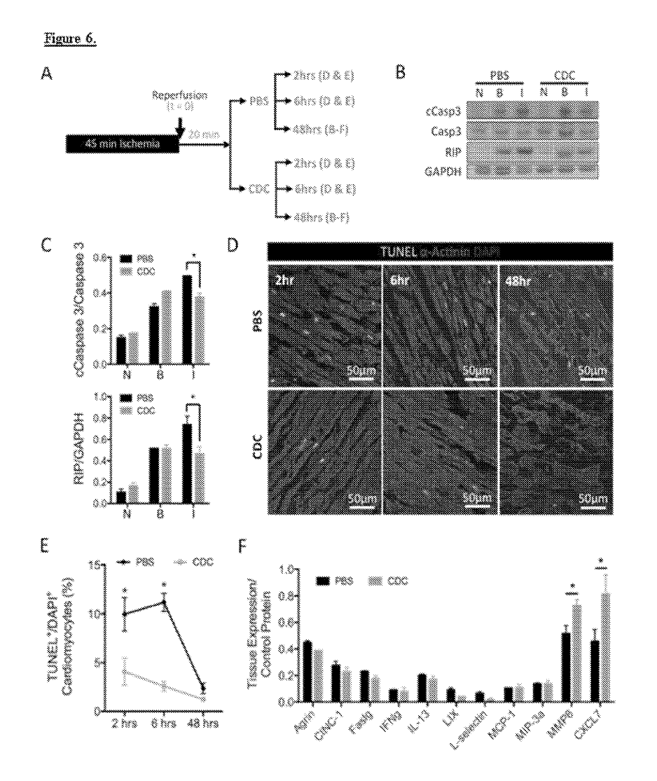

The observed reduction in infarct mass may reflect, at least partially, a reduction in programmed cardiomyocyte death. To test this hypothesis, the Inventors probed cell death in the infarct (I), border (B), and normal (N) zones at various times (FIG. 6A) and observed a reduction in cleaved caspase 3 and RIP proteins within the infarct tissue (FIG. 6A-C). CDC-treated hearts showed reduced TUNEL-positive cardiomyocytes within the infarct region (FIG. 6D), most dramatically at 2 and 6 hours post-IR (FIGS. 6D & 6E). Cytokine protein arrays revealed elevated protein expression of MMP8, which has been associated with wound healing and Mφ inactivation, and CXCL7, which is inducibly expressed in monocytes in response to stromal stimulation (FIG. 6F). These were the first hints that Mφ might be involved in the cardioprotective effect of CDCs.

### Example 21

### CDCs Reduce the Number of CD68⁺ Macrophages within the Ischemic Heart

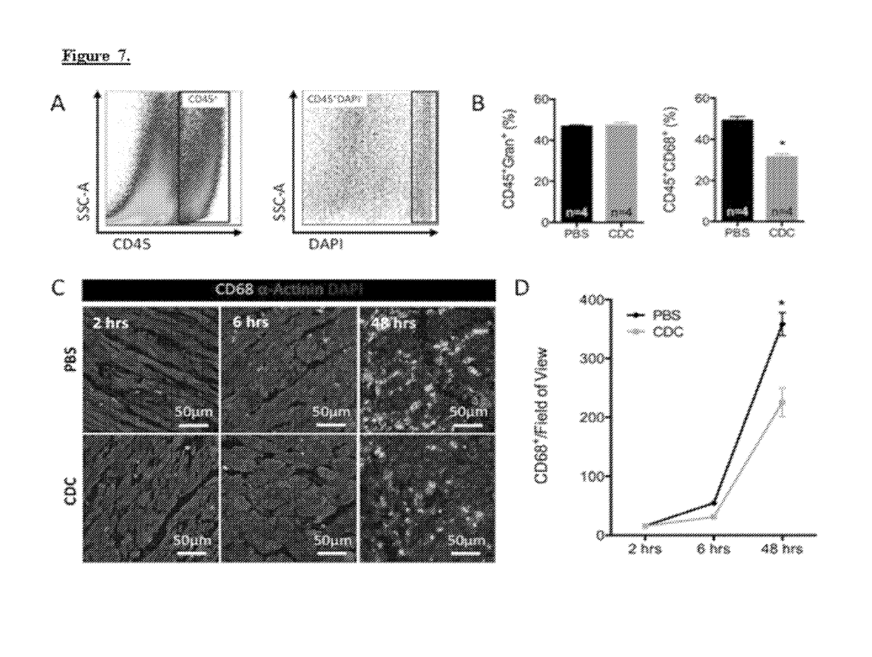

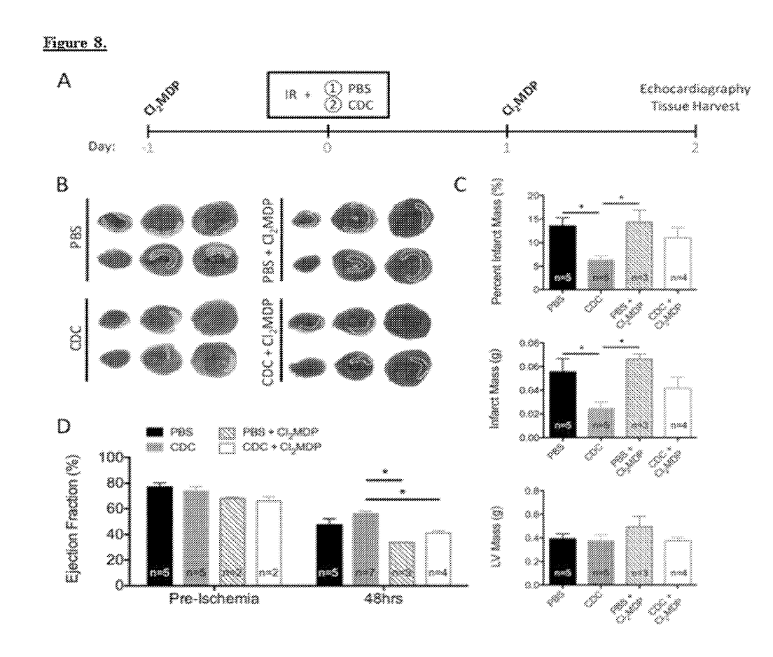

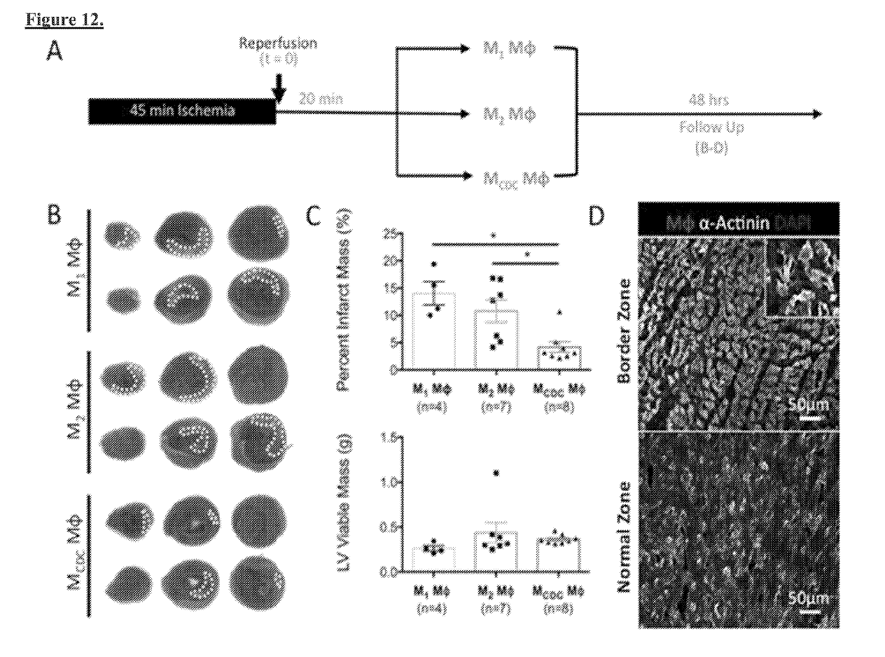

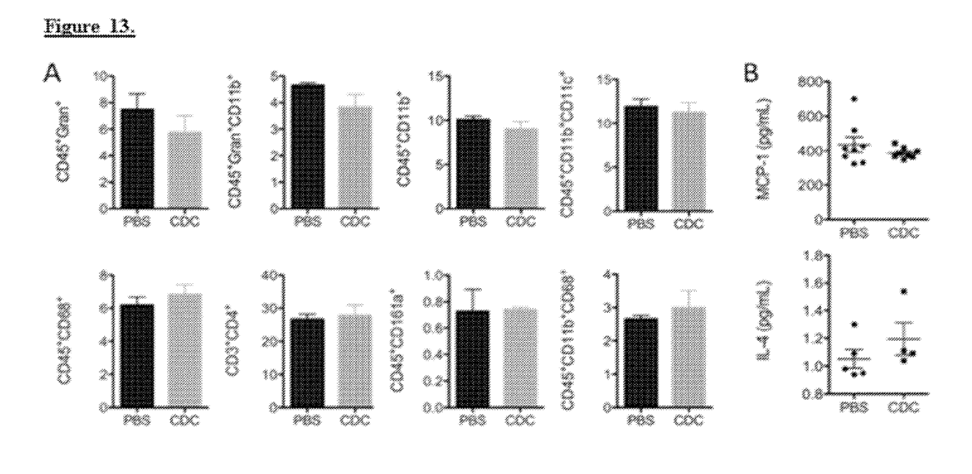

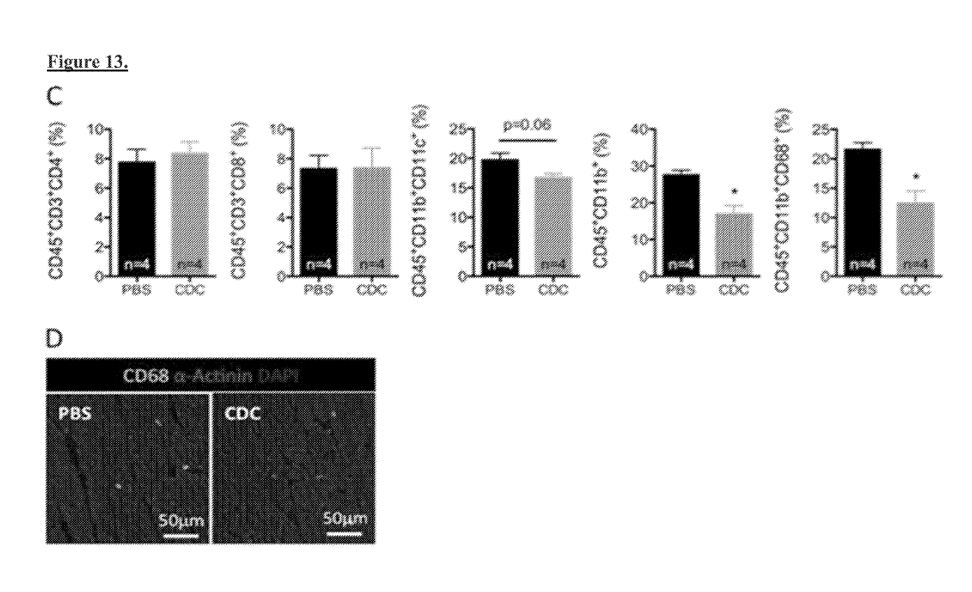

To test the hypothesis that CDCs modulate inflammation following IR injury, the Inventors examined the leukocyte profile from peripheral blood and cardiac tissue (FIG. 7A). Delivery of CDCs to the heart altered neither circulating leukocytes (FIG. 13A) nor serum expression of proinflammatory MCP-1 or IL-4 (FIG. 13B). It did, however, reduce specific leukocyte populations within the heart, notably CD45+CD68⁺ M (FIG. 7B) and CD45⁺CD11b⁺CD11c⁺ dendritic cells (FIG. 13C); both are members of the mononuclear phagocyte (MNP) system. Interestingly, the granulocyte population (CD45⁺Gran⁺), which is another significant acute infiltrating inflammatory cell type, was unaltered (FIG. 7B). These data were validated using immunohistochemistry to detect CD68 within the infarct region of hearts isolated at 2, 6, and 48 hours following IR (FIGS. 7C & 7D). While the Inventors observed similar levels of CD68 expression in sham (FIG. 13D) and hearts treated with CDC or PBS at 2 and 6 hours post-IR, at 48 hrs there was a significant reduction in the number of CD68⁺ cells (Mφ) in the hearts of CDC-treated animals (FIGS. 7C & 7D).

### Example 22

### Systemic Depletion of Mφ Using Clodronate (Cl₂MDP) Reduces the Efficacy of CDC Therapy

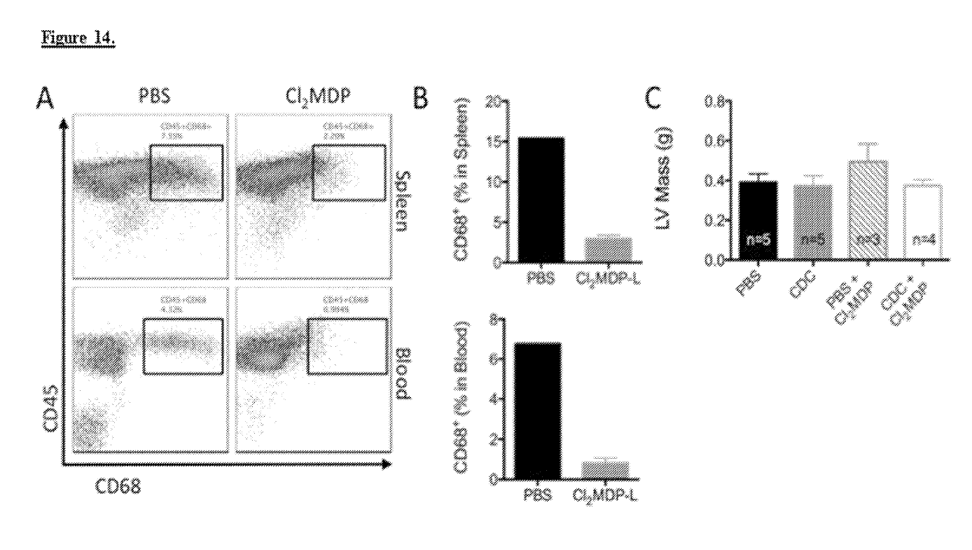

Since the Inventors observed a decrease in Mφ counts within the infarcted myocardium, the Inventors tested whether systemic depletion of Mφ would recapitulate the benefits of CDC therapy. To do so, the Inventors administered clodronate (Cl₂MDP—dichloromethylene diphosphonate) liposomes 24 hours prior to and following IR injury (FIG. 8A). As expected, clodronate reduced systemic Mφ populations (FIGS. 14A & 14B). The Inventors first assessed infarct size as an indicator of bioactivity. Clodronate itself did not aggravate IR injury, as the Inventors observed no significant difference in infarct mass between PBS and PBS+Cl₂MDP groups. However, clodronate attenuated the benefits of CDCs: infarct mass was greater in CDC+Cl₂MDP relative to the CDC-treated group (FIG. 8B & FIG. 8C). The functional changes mirrored those observed pathologically, in that no significant difference in ejection fraction was observed between PBS and CDC+Cl₂MDP 2 days post-IR (FIG. 8D). Thus, the Inventors rejected the hypothesis that Mφ depletion would recapitulate the benefits of CDC therapy. Instead, the data indicate that Mφ are required for the cardioprotective effects of CDC therapy. The Inventors thus went on to investigate, in detail, potential effects of CDCs on Mφ phenotype and function. As the CD45⁺CD68⁺ Mφ population showed the greatest changes in post-MI tissue (FIG. 7B), the Inventors decided to focus on the role of this subpopulation in cardioprotection.

### Example 23

### CDCs Shift the Cardiac CD68⁺ Mφ Population Away from an M₁ Phenotype In Vivo

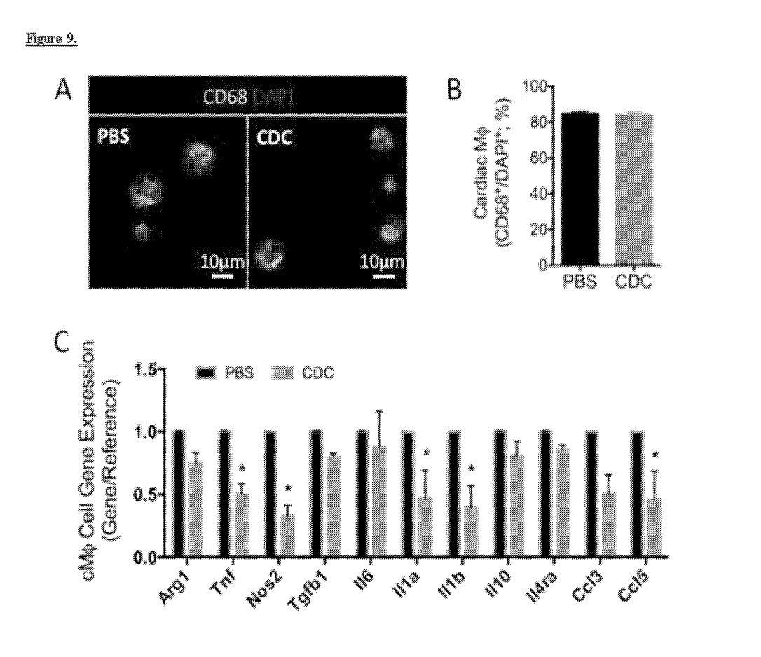

Macrophages are well-recognized to exhibit the capacity to polarize between M₁ and M₂ phenotypes. The M₁ population is generally defined by its early infiltration into the myocardium and proinflammatory cytokine expression (e.g. Nos2, Tnf Il1b, and Il6), while the M₂ population is associated with resolution of late-phase inflammation and promotion of tissue repair (e.g. Arg1, Il10, and Pparg). The Inventors therefore asked whether CDCs polarize Mφ toward the M₁ or M₂ phenotype. To do so, the Inventors created MI by permanently ligating the left anterior coronary artery and randomly allocated rats to receive 2×10⁶ CDCs or an equivalent volume of vehicle (PBS) through 4 direct injection sites in the infarct border zone. Two days later, hearts were harvested and the infarct and surrounding border tissue were digested. The resulting cell suspension was separated using a density gradient to isolate the mononuclear cell fraction and then cardiac Mφ (cMφ) were purified by attachment on cell culture dishes (FIGS. 9A & 9B). The >85% pure CD68⁺ populations were then analyzed by qRT-PCR for M₁ and M₂ gene expression markers (FIG. 9C). Interestingly, M₁ markers Nos2, Tnf, and Il1b were significantly reduced, but there was no concomitant increase in M₂ markers such as Arg1, Il10, or Il4Ra. These data indicate that CDCs reduce the number of CD68⁺ macrophages in the infarcted myocardium and polarize macrophages away from the M₁ phenotype, but not towards a classical M₂ state.

### Example 24

### CDCs Polarize Thioglycollate Activated Mφ Away from an M₁ Phenotype In Vitro

To test whether CDCs have the capacity to modulate Mφ polarity indirectly, the Inventors devised an in vitro transwell co-culture protocol (FIG. 15A). With limited Mφ yield from cardiac tissue, the Inventors utilized Mφ derived from the peritoneal cavity following thioglycollate-stimulation, which are readily available and highly pure. Although these peritoneal Mφ (pMφ) are partially activated, the Inventors sought to examine whether CDCs could shift their activation profile away from a proinflammatory state.

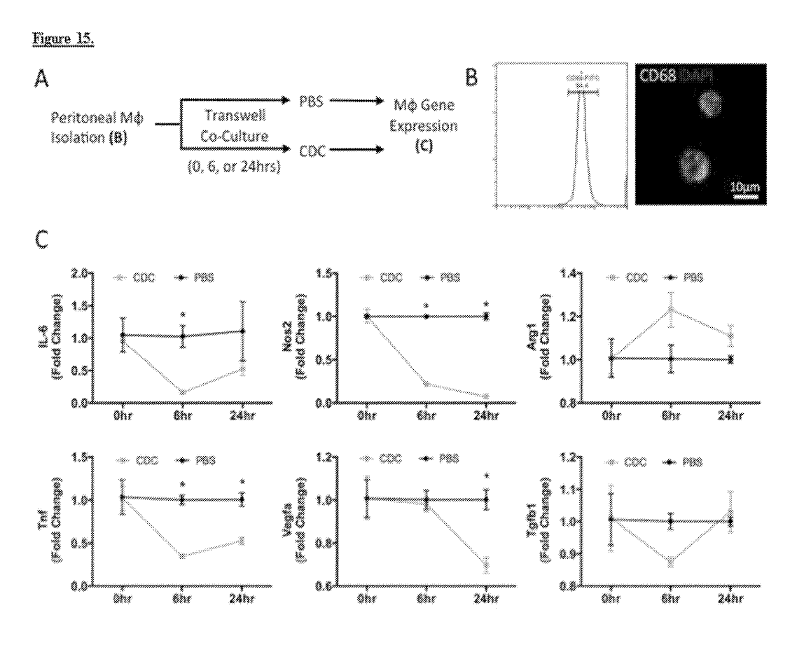

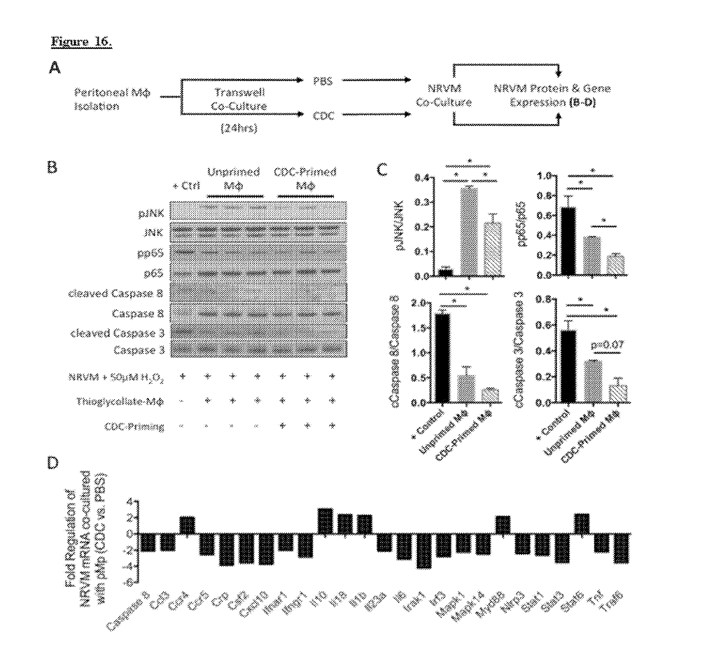

A process of peritoneal lavage, RBC lysis, and attachment to cell culture plates yielded a highly pure (>90%) CD68⁺ mononuclear cell population (FIG. 15B). Peritoneal Mφ were then pre-incubated with CDCs, in a transwell co-culture system, or PBS. After 6 hours of incubation, CDC-primed pMφ exhibited reduced M₁ gene expression (Il16, Nos2, and Tnf), without any significant changes in Arg1, Vegfa, or Tgfb1 (FIG. 15C). Although the gene expression profile was consistent with a reduction in cytotoxic, proinflammatory cytokines, the Inventors wanted to test the hypothesis that CDC-primed pMφ confer cytoprotection toward distressed ventricular cardiomyocytes without direct cell contact. To do so, the Inventors exposed neonatal rat ventricular myocytes (NRVMs) to 50 μM H₂O₂ then co-cultured them in a transwell system with CDC- or PBS-primed pMφ to simulate an in vivo oxidative stress IR-relevant environment (FIG. 16A). After 6 hours, the Inventors observed, in NRVMs that had been transwell co-cultured with CDC-primed pMφ, reductions in phospho-JNK, phospho-p65 activity, cleaved caspase 3 and caspase 8 activity (FIGS. 16B & 16C). Gene expression profiling generally corroborated the protein data. Although not all NRVM genes were concordant, the directional change of a large proportion suggested a protective phenotype, including reduced expression of TLR signaling mediators (Traf6, Irak1, Irf3) and proinflammatory cytokines (Crp, Il23a, Il6, Nlrp3, Tnf) (FIG. 16D).

### Example 25

### CDCs Polarize Unstimulated Bone Marrow (BM)-Derived Mφ Toward a Phenotype (M_{CDC}) Distinct from Either M₁ or M₂

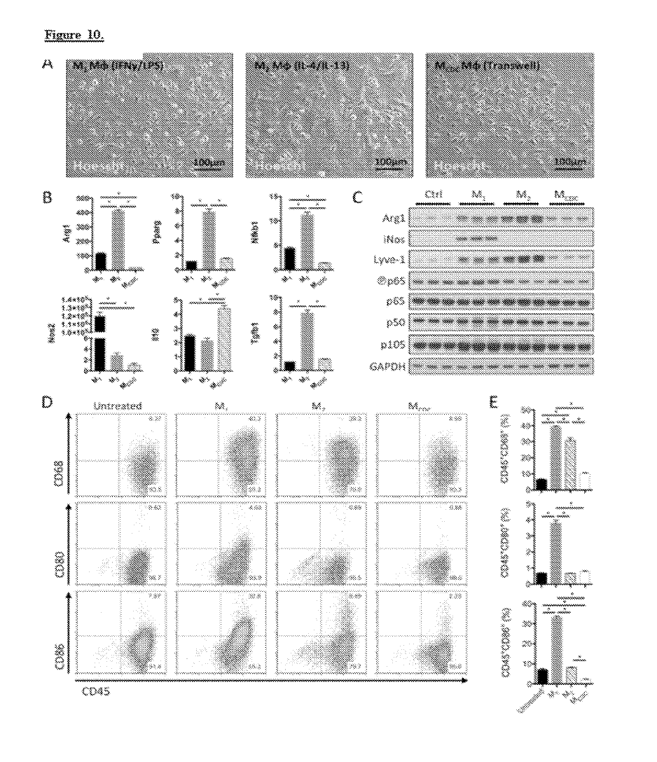

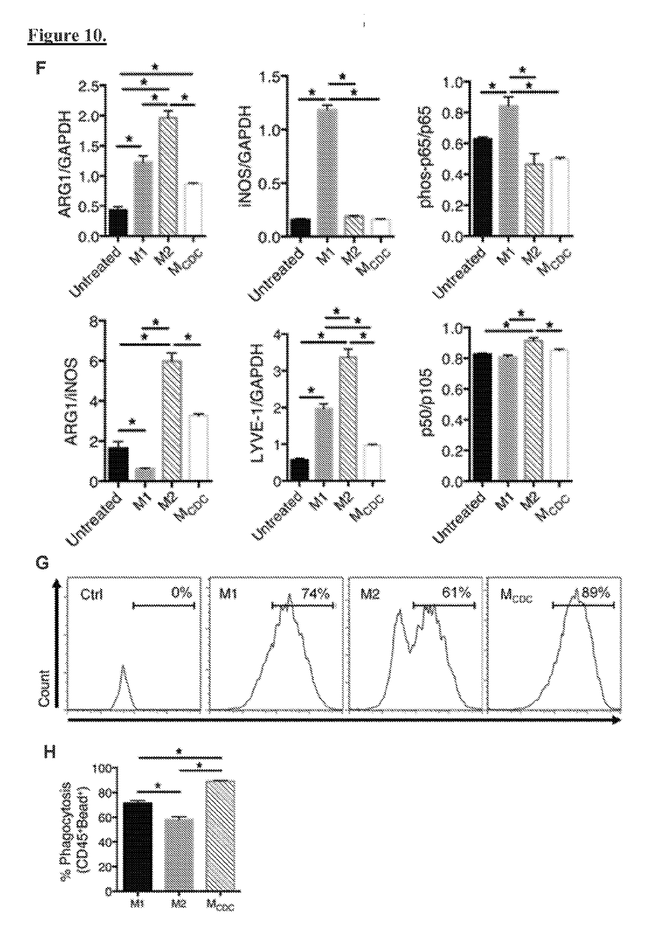

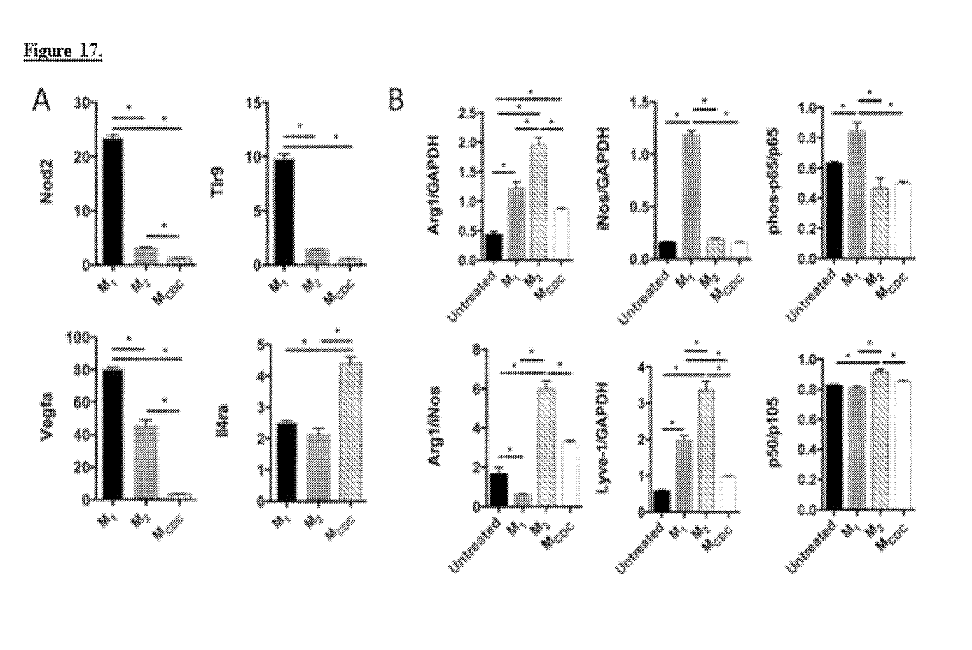

Following ischemic insult, monocytes are actively recruited from both splenic and BM reserves and subsequently differentiate into Mφ at the site of injury. To recapitulate the in vivo recruitment of naïve/unprimed monocytes to the site of injury in vitro, the Inventors isolated BM cells from femurs, cultured the cells with M-CSF, then differentiated them into M₁ (IFNg & LPS), M₂ (IL-4 & IL-13), or M_{CDC} (CDC transwell) Mφ (FIG. 10A). To examine whether M_{CDC} Mφ were similar or distinct from M₁ or M₂ Mφ the Inventors compared the gene expression profiles for known M₁ and M₂ genes (FIG. 10B) and general Mφ markers (FIG. 17A). As expected, M₁ Mφ had elevated Nos2, while M₂ Mφ had higher Arg1 and Pparg, expression relative to untreated Mφ. M_{CDC} Mφ, on the other hand, had reduced Nos2 and Arg1 relative to both M₁ and M₂, indicating that they were polarized to neither a true M₁ nor an M₂ state. Of all genes examined, M_{CDC} Mφ expressed the highest level of Il10.

Two well-established markers for M₁ and M₂ Mφ polarity are Nos2 and Arg1, respectively. The divergent phenotypes involve a common metabolic pathway that converts L-arginine to either L-citrulline and nitric oxide (Nos2 catalysis) or L-ornithine and urea (Arg1 catalysis). Therefore the Inventors examined the relative ratio of protein expression of Nos2 and Arg1. As expected, M₂ Mφ exhibit the largest Arg1/Nos2 ratio, as well as Lyve-1, and p50 expression, whereas M₁ Mφ have the lowest Arg1/Nos2 ratio, as well as elevated phospho-p65 expression (FIG. 10C & FIG. 17B). Interestingly, M_{CDC} Mφ have several intermediate protein expression patterns, exhibiting an Arg1/Nos2 ratio between M₁ and M₂, slightly elevated Lyve-1 relative to untreated, low phospho-p65 (similar to M₂), and low p50 expression (similar to M₁) (FIG. 10C & FIG. 17B). Flow cytometric analyses of M_{CDC} Mφ reveal a reduction in cell size relative to M₁, M₂, or unstimulated BMDMs, as well as distinct expression of surface markers CD68, CD80, CD86, CD11b, CD45, and FSC (FIG. 10D, 10E, & FIG. 18).

### Example 26

### M_{CDC} Mφ Reduce Apoptosis in Oxidatively-Stressed Cardiomyocytes In Vitro

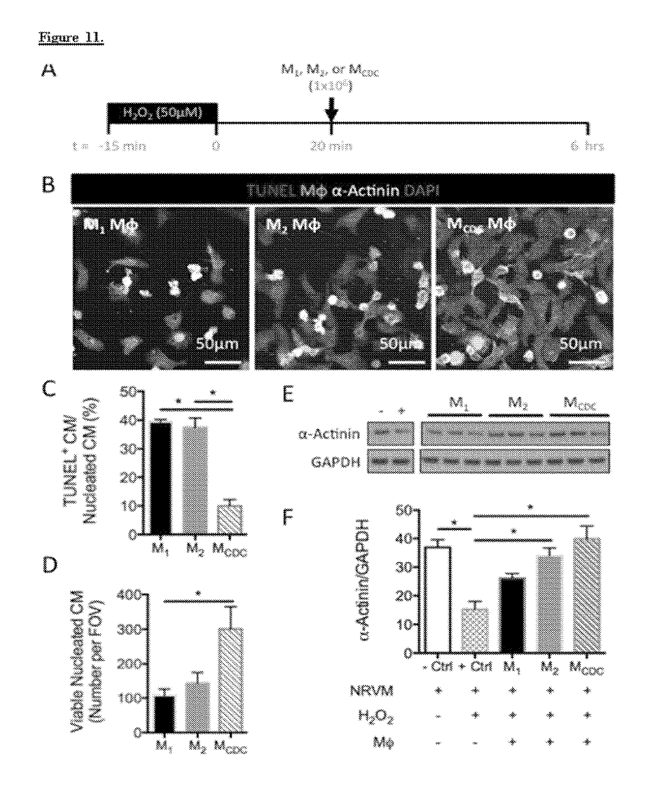

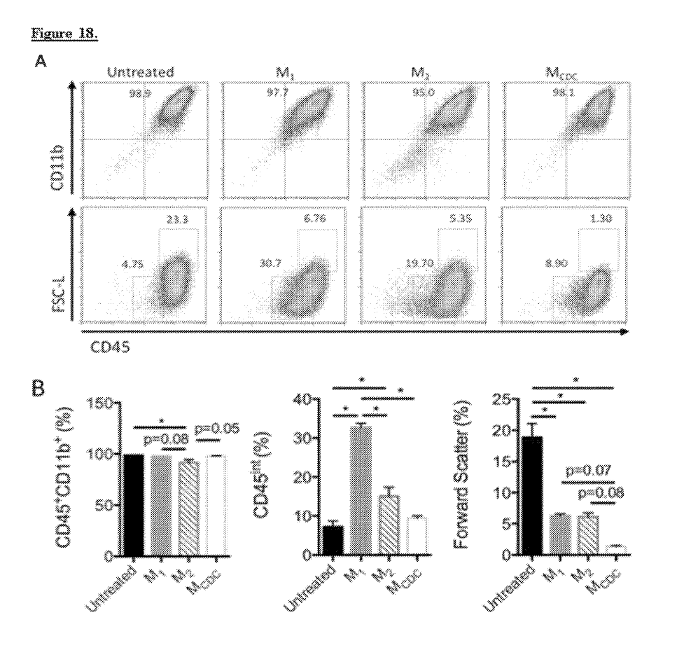

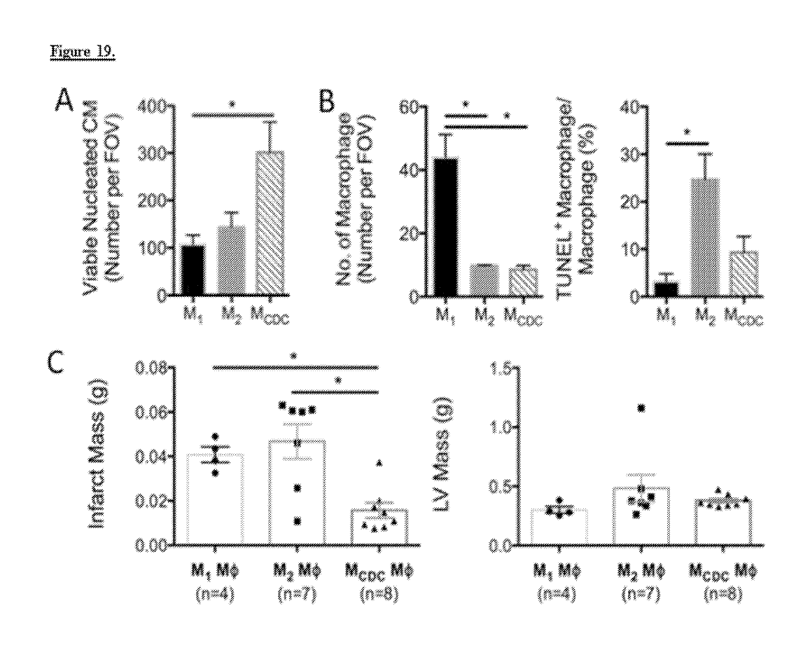

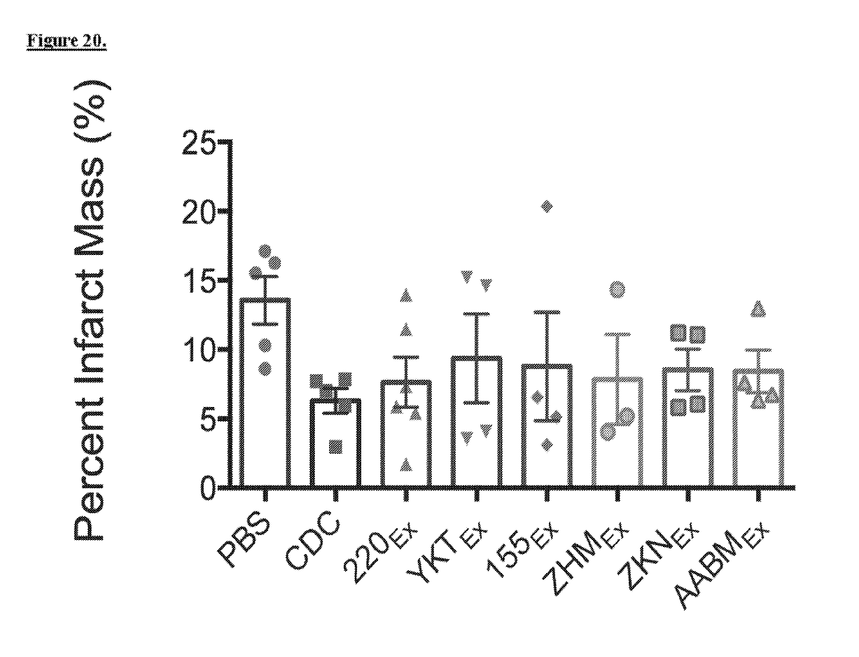

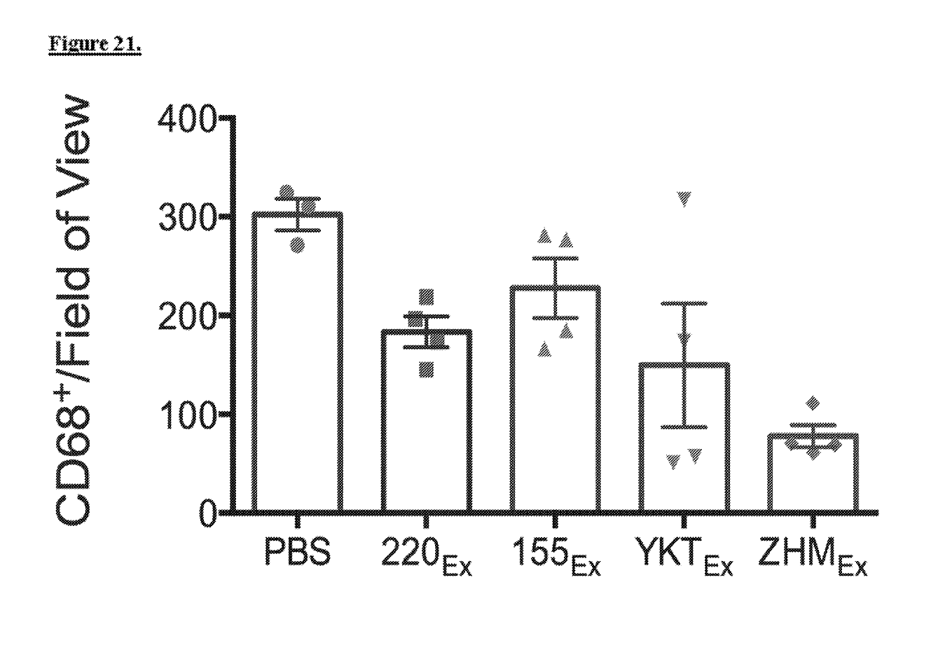

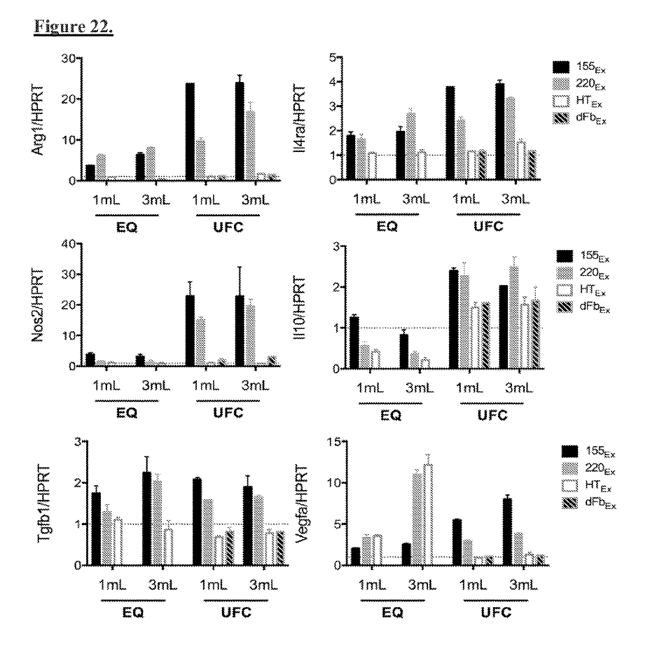

The recruitment of Mφ to a site of injury results in the phagocytosis of cellular debris and expression of an array of cytokines. As the Inventors' in vitro gene expression analyses suggested that M_{CDC} Mφ secrete a unique cytokine profile, the Inventors sought to examine if M_{CDC} Mφ are protective to stressed cardiomyocytes. In in vitro co-culture, NRVMs were stressed with 50 μM H₂O₂ prior to the addition of DiO-labeled M₁, M₂, or M_{CDC} Mφ (FIG. 11A). Cells were examined for viability and number following 6 hours of co-incubation. Interestingly, the addition of M_{CDC} Mφ to H₂O₂-treated NRVMs significantly reduced TUNEL⁺ cardiomyocytes and preserved viable cardiomyocytes relative to M₁ or M₂ Mφ (FIG. 11B-11F & FIG. 19A). Interestingly, at the end of the co-culture period the Inventors observed differences in the remaining number of Mφ. The Inventors found a greater number of M₁, relative to M₂ or M_{CDC}, Mφ and increased Mφ TUNEL positivity in M₂, relative to M₁ or M_{CDC}, Mφ (FIG. 19B). Thus, in an oxidative stress model, M_{CDC} Mφ do not themselves undergo significant apoptosis, but rather limit bystander cardiomyocyte apoptosis. The Inventors therefore tested whether M_{CDC} Mφ confer cardioprotection in vivo.

### Example 27

### Adoptive Transfer of M_{CDC}, but not M₁ or M₂, Mφ Simulate CDC Therapy Post-IR

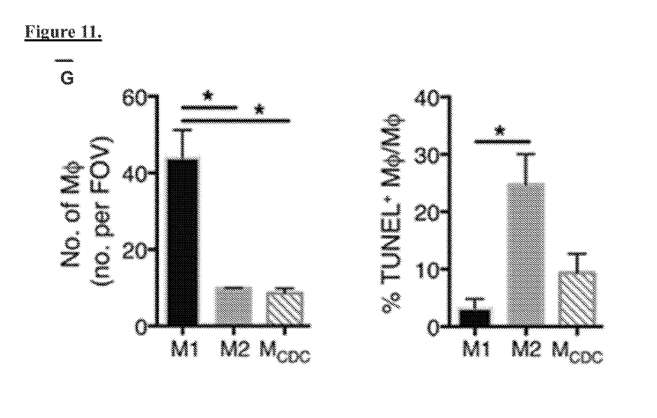

The Inventors used adoptive transfer to examine whether M_{CDC}s could recapitulate the benefits of CDCs in vivo. To minimize confounding effects of endogenously-recruited Mφ, the Inventors focused on the time window where low levels of Mφ were present in the infarcted myocardium (up until 6 hours post IR) (FIG. 7C). Therefore, the Inventors devised a delivery protocol similar to that described earlier in the study, but infusing polarized Mφ (M₁, M₂, or M_{CDC}) rather than CDCs 20 minutes post-IR (FIG. 12A). All Mφ were labeled with DiI to trace the cells following delivery. After 48 hours of reperfusion, M_{CDC}-treated animals had preserved cardiac function, as well as reduced infarct mass relative to M₁ and M₂ Mφ-treated animals (FIG. 12B-C & FIG. 19C). These Mφ were localized to the border zone and observed in high frequency (FIG. 12D).

### Example 28

### Cardioprotective Effects of CDCs as Mediated Via Mφ

Prolonged myocardial ischemia leads to a progressive wave-front of cell death beginning within the subendocardium and extending toward the epicardium. The gold standard of therapy for acute MI is percutaneous intervention with the aim of opening the occluded vessel as soon as possible to reduce cell death. Nevertheless, reperfusion itself confers some injury to the myocardium. Several strategies have been employed in efforts to reduce the detrimental effects of IR, including ischemic pre-conditioning, but pretreatment is required, limiting realistic utility in MI patients. A more clinically-tractable strategy includes ischemic post-conditioning, whereby brief cycles of ischemia imposed during early reperfusion can reduce infarct size, but, without immediate manipulation of flow at the time of reperfusion, benefit is lost. The discovery that CDCs work in MI despite having been administered with some delay after reperfusion is notable: no other cardioprotective modality successfully reduces IS without pretreatment and/or immediate intervention upon reopening the affected artery. The idea that cell therapy may mitigate ischemic injury by modulating Mφ is also conceptually novel, but consistent with the immunomodulatory properties recently described for CDCs.

Cell therapy is under development as an adjunctive strategy to treat ischemic heart disease. The focus, however, has been on subacute or chronic stages of myocardial injury, at which time the opportunity for myocardial preservation has long passed. Here, the Inventors have demonstrated that infusion of CDCs into the coronary circulation 20 minutes post-IR confers profound cardioprotection to the damaged myocardium. This effect is lost, however, if CDC infusion is delayed to 2 hours post-IR. This finding is consistent with the observation that reperfusion injury increases over time and in parallel with microvascular obstruction, which may physically restrict efficient transport of CDCs through the coronary circulation. Inflammation is a critical, but poorly understood facet of IR injury. With evidence supporting an immunomodulatory role for CDCs in vitro and in a chronic model of MI, the Inventors hypothesized that CDCs would modify the local, innate immune response to confer cardioprotection following IR.

Macrophages are a populous and highly plastic immune cell source. During the acute phase of inflammation, these cells are found either endogenously within tissues as resident Mφ (e.g. skin, brain, liver, and heart), or peripherally recruited from BM or splenic reserves, as inflammatory Mφ. Within the heart, at least 4 populations exist at steady state. During an inflammatory reaction, such as ischemia, Ly6^{hi} monocytes are rapidly recruited to the site of injury within the heart and support the replenishment of resident cell subpopulations. Here, the Inventors demonstrate in Mφ from three distinct sources (cardiac, peritoneal, and BM-derived) that CDCs specifically shift Mφ away from a proinflammatory (M₁-like) phenotype. With a low retention rate following coronary infusion, the Inventors propose that CDCs secrete factors that foster a cardioprotective microenvironment with extensive crosstalk between resident and infiltrating cell types necessary for repair.

Tissue microenvironments have been well described and studied over the past several decades, most notably within the BM and tumor microenvironments. These distinct niches support not only normal stem cell function and therapeutic activation, but also malignancy. Recent data suggest that inflammatory cells, and most specifically Mφ which exist in close proximity to stromal cells and resident stem cells, are essential in maintaining the hematopoietic stem cell (HSC) niche. It is likely that Mφ and stromal cells bi-directionally communicate to support repair in several different tissue microenvironments. For instance, Mφ are necessary to form the niches required for limb regeneration in salamanders, for skeletal muscle regeneration following toxin-mediated injury, and for cardiac regeneration in neonatal mice post-MI. With a growing appreciation for Mφ heterogeneity, including M₁ and M₂ subpopulations, it will be important to delineate the factors governing the polarization (denoted by transcriptional regulation and expression of surface markers) of resident tissue as well as recruited inflammatory Mφ during ischemic injury. Since resident and inflammatory Mφ derive from distinct progenitor sources (yolk sac- versus BM-derived), it is likely that each population is intrinsically distinct but endowed with a capacity to serve functionally redundant roles, as demonstrated through the repopulation of resident cardiac Mφ with Ly6C^{hi} monocytes following ischemic injury.

The Inventors' results provide novel insight into the mechanism of cellular therapy following ischemic injury. As a result of CDC-induced Mφ priming, the Inventors confer the ability to drive Mφ toward a cytoprotective state. Specifically, the Inventors demonstrate the capacity to not only reverse the preactivated, thioglycollate-stimulated pMφ away from a cytotoxic phenotype (simulating activated Mφ within the myocardium), but also the directed transition from a more naïve state, as observed in the Inventors' BM-derived Mφ experiment (simulating recruited Mφ within the myocardium), toward a cytoprotective phenotype. Mφ primed by CDCs exhibit a distinctive polarization state characterized by the expression of specific genes, cytokines, and membrane markers, which together confer cytoprotective properties. M_{CDC}s infused post-IR have the endogenous capacity to home to the ischemic border zone, where they reduce infarct size.

The finding of a novel, Mφ-mediated mechanism of cardioprotection highlights the protean effects of heterogeneous Mφ populations. By favoring one particular polarized Mφ state, CDCs confer therapeutic efficacy when administered after reperfusion, a setting previously believed to be refractory to medical intervention. The present work defines a promising intervention, targeted at the inflammatory cascade, which can limit myocardial injury. The Inventors not only pinpoint Mφ as the key effectors of CDC-induced cardioprotection, but also find that Mφ themselves, when appropriately primed, can have therapeutic utility. The fact that functional and structural benefits can be recruited 20 min after reperfusion, a time when previous work would suggest that the cascade of death is set in stone, is noteworthy.

### Example 29

### CDC-Derived Exosomes and Mφ Polarization

As described, the above results suggested CDCs secrete factors that foster a cardioprotective microenvironment with extensive crosstalk between resident and infiltrating cell types necessary for repair. Extending these observations, CDC-derived exosomes reproduce CDC-induced therapeutic regeneration, and that inhibition of exosome production undermines the benefits of CDCs. Exosomes contain microRNAs, which have the ability to alter cell behavior through paracrine mechanisms.

MicroRNAs, such as miR-146a appear to play an important part in mediating the effects of CDC exosomes, but alone may not suffice to confer comprehensive therapeutic benefit. Other microRNAs in the repertoire may exert synonymous or perhaps synergistic effects with miR-146a. For instance, miR-22 (another microRNA highly enriched in CDC exosomes) has been shown to be critical for adaptive responses to cardiac stress. Likewise, miR-24 (also identified in CDC exosomes) modulates cardiac fibrosis by targeting furin, a member of the profibrotic TGF-b signaling pathway; overexpression of miR-24 in a model of MI decreased myocardial scar formation. The possible roles of these microRNAs as mediators of CDC exosome benefits, alone or in combination with miR-146a, remain to be studied. Whereas dissection of the active principles within CDC exosomes is worthwhile, deconstruction of the nanovesicles may be counterproductive from a therapeutic perspective. CDC exosomes are naturally cell permeant, and their lipid bilayer coat protects their payloads from degradation as particles shuttle from cell to cell, so that the intact particles themselves may be well suited for disease applications.

As reported by Ibrahim et al., injection of CDC-derived exosomes into the injured heart can mimics the structural and functional benefits of CDC transplantation; conversely, inhibition of exosome secretion by CDC s abrogates the therapeutic benefits of transplanted CDC s. Not all exosomes are salutary: Injection of exosomes from dermal fibroblasts, control cells which are therapeutically inert, had no benefit. DC-exosomes decreased acute cardiomyocyte death and inflammatory cytokine release, while attenuating left ventricular (LV) remodeling and fibrosis in the injured heart. MicroRNA arrays reveal several “signature microRNAs” that are highly up-regulated in CDC-exosomes. In contrast, mass spectrometry indicates that the protein composition of CDC-exosomes is conventional and comparable to that of fibroblast exosomes.

Stem cell derived exosomes, and the microRNAs they contain, as crucial mediators of regeneration. CDCs exert diverse but coordinated effects: they recruit endogenous progenitor cells and coax surviving heart cells to proliferate; on the other hand, injected CDCs suppress maladaptive LV remodeling, apoptosis, inflammation, and tissue fibrosis after MI. In the context of PAH, similar benefits are likely to exist in the repair and remodeling of microvasculature.

While it is possible that CDCs secrete a medley of individual growth factors and cytokines that collectively produce diverse benefits, the involvement of master-regulator microRNAs within exosomes would help tie together the various effects without postulating complex mixtures of numerous secreted protein factors. Moreover, microRNAs are known to confer long-lasting benefits and fundamental alterations of the injured microenvironment helping to rationalize the sustained benefits of CDCs despite their evanescent survival in the tissue. CDC exosomes contain rich signaling information conferred by a cell type that is the first shown to be capable of producing regeneration in a setting of “permanent injury”, and confer the same benefits as CDCs without transplantation of living cells.

Here, described study further establishes that exosomes possess significant potency in modulating regeneration and repair mechanisms, as capable of transferring the salutary benefits to cells that are otherwise therapeutically inert. Of great interest would be pinpointing the cargo contents responsible indispensable for imparting such therapeutic benefits, whether growth factors, cytokines, “shuttle RNA” such as microRNAs, or other factors. Identification of such factors would eventually lead to opportunities for generating wholly synthetic exosomes, containing the same or substantially similar set of factors enriched in therapeutically effective cells such as CDCs.

Based on the results described herein, CDC-exosomes are demonstrated as capable of treating pulmonary and heart-related conditions. Exosomes secreted by cells possess the cargo contents capable of reproducing therapeutic benefits of their parental cells. Importantly, these results have further identified that within their rich biological cargo of various proteins and RNA, microRNAs play a central role in activating regenerative processes, suggesting compelling applications in clinical therapeutics. Exosomes have significant advantages over traditional cell-based therapies including manufacturing advantages, relative ease of definition and characterization, lack of tumorigenicity and immunogenicity, and possibility of administration in therapeutic scenarios for which cell, tissue, organ or mechanical transplant is not available. Thus, CDC-exosomes represent a significant advance biologic therapy.

### Example 30

### Paracrine Effects of Exosomes

Multicellular self-assembling cardiospheres (CSps) exert regenerative and antifibrotic effects via paracrine mechanisms. There is increasing evidence that cardiosphere-derived cells (CDCs) mediate most or all of the beneficial therapeutic effects via secreted exosomes. Of great interest is deciphering the target recipient cells of secreted exosomes, the genotypic and phenotypic alterations occur upon receipt and transfer of cargo contents, and the scope of such alterations in the processes of regeneration and repair. In establishing an animal model evaluation exosome alteration capacity, the Inventors established a rat model of chronic myocardial infarction measuring the effects CSp-secreted exosomes. The Inventors also sought to determine if CSp-exosomes could convert the phenotype of therapeutically inert cells, a finding which can begin to decipher the complex array of cellular actors ultimately involved in regeneration and repair processes.

Wistar Kyoto rats with permanent LAD ligation were subject to repeat thoracotomy one month post-myocardial infarct (MI) and intramyocardial injection of (a) human dermal fibroblasts (DFs), (b) CSp exosomes (c) DFs primed with CSp-exosomes (d) CSps only or (e) vehicle. Functional and histological analyses were performed 4 weeks after therapy. Mechanisms were also probed in vitro. Exosomes were readily isolated from CSp-conditioned media by adding a precipitation solution followed by centrifugation. Confocal imaging revealed internalization of fluorescently labeled CSp-exosomes in rat DFs that had been incubated with CSp-exosome for 24 hours in culture. In vitro, exosome primed DFs increased tube formation by human umbilical vein endothelial (HUVEC) cells and cardiomyocyte survival as compared to unprimed DFs.

### Example 31

### Therapeutic Benefits as Mediated by Exosome Alteration of Non-Therapeutic Fibroblasts

In vivo, one month post therapy, CSp-exosomes alone and CSPs-only equally increased cardiac function and reduced scar mass compared to the vehicle and DFs injected groups (EF=45±1.1% in the CSp-exosome, 44±1.6% in the CSPs, 32.7±1% in the placebo and 34.8±1.7% in the DF group, p<0.01 by one way Anova; scar intramyocardial engraftment 1 hour post injections. Interestingly, the exosome primed DFs revealed enhanced regenerative capacity compared to the unprimed group (EF=41±1 in the primed-DF group, p=0.05 compared to unprimed DFs and car mass=49.5 mg in the primed-DF group, p<001 vs. the unprimed DFs). Immunocytochemistry showed increased vessel density in animals injected either with CSp or CPS-exosome or exosome primed-DFs compared to the other two groups.

These findings demonstrate that administration of CSp-exosomes recapitulates the regenerative potential and functional benefits of CSPs themselves. More importantly, these cell-free lipid bilayer nanovesicles conferred therapeutic efficacy on inert DFs, a finding that hints at an unanticipated amplification mechanism for exosome-mediated therapeutic benefits, wherein salutary benefits of exosome administration can be conferred upon cells that are otherwise therapeutically inert. This finding demonstrates exosomes as not only capable of effectuate regeneration and repair mechanisms, but exhibit significant potency in modulating these effects.

### Example 32

### Methods

Female Wistar Kyoto rats (n=54) 5 to 6 weeks of age were used for in vivo experiments. Human dermal fibroblasts (hDFs), human cardiospheres (hCSps), and human cardiosphere-derived extracellular vesicles (hCSp-EMVs) were used for in vitro assays. To avoid any possible confounding effects of xenotransplantation, the Inventors used rat DFs (rDFs) and rat CSp-EMV (rCSp-EMVs) for in vivo experiments.

To create MIs, animals underwent permanent left anterior descending artery ligation. Four weeks later they underwent a second survival thoracotomy with animals randomly assigned to intramyocardial border zone injection using 1 of 4 treatments: (1) rCSp-EMV-derived from 2 mol/l cells (n=16); (2) 2 mol/l rDFs (n=12); (3) 2 mol/l rDFs incubated overnight with rCSp-EMVs, then washed (rCSp-EMV DF, n=16); or (4) vehicle (phosphate-buffered saline [PBS]; n=10). The animals were monitored for an additional 4 weeks followed by endpoint functional and histological studies.

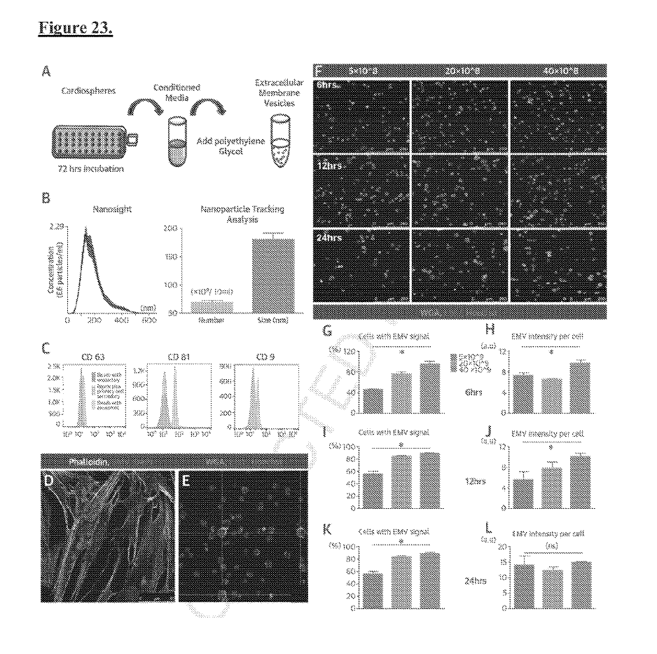

Baseline transthoracic echocardiography was performed 28 days post-MI (2 days before the second thoracotomy). Briefly, long-axis images were used to measure left ventricular end-systolic and end diastolic volumes and ejection fraction. Short-axis M-mode images at the level of the papillary muscle were used to measure end-systolic diameter. Follow-up echocardiographic analysis was performed 4 weeks post-injections followed by euthanasia. CDCs were isolated from male Sprague Dawley and Brown Norway rats and cultured in Iscove's Modified Dulbecco's Medium (IMDM; Life Technologies, Carlsbad, Calif.) supplemented with 20% fetal bovine serum and antibiotics. To form cardiospheres, 15 mol/l CDCs were incubated with IMDM supplemented with penicillin/streptomycin and 0% fetal bovine serum in ultra-low attachment dishes. Three days later, the conditioned medium was collected and processed for EMV isolation (FIG. 23A).

For statistical analysis, pooled data are expressed as means±SE. Statistical analysis was performed using factorial analysis of variance followed by a Tukey post-hoc analysis of mean differences or with paired Student t test, indicated in figures by lines connecting compared values. A value of p≦0.05 was accepted as significant.

### Example 33

### EMV Characterization and Internalization

EMVs were isolated from serum-free medium conditioned by hCSps over a period of 3 days. The final pellet contained 12×10⁹/ml of 175±12-nm diameter vesicles by nanoparticle tracking analysis (NTA; NanoSight Ltd., Amesbury, Wiltshire, United Kingdom) (FIG. 23B). Flow cytometry revealed that these vesicles expressed tetraspanins characteristic of exosomes such as CD63, CD9, and CD81 (FIG. 23C).

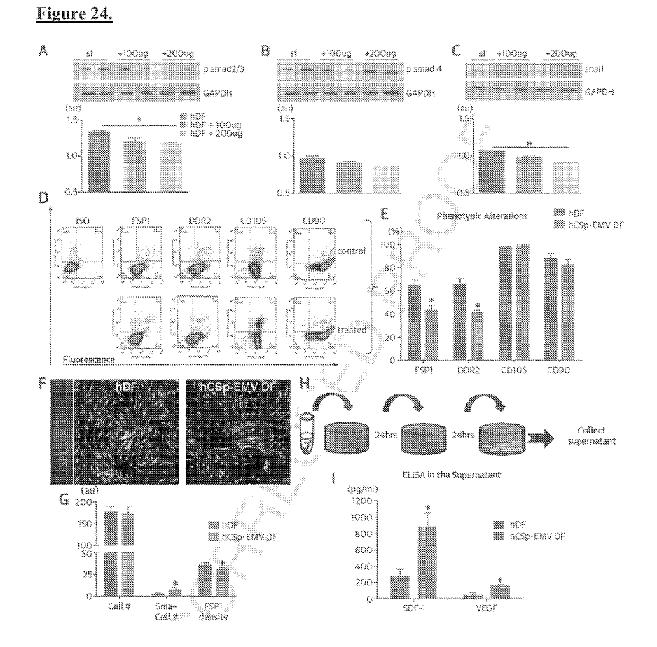

Adding the final pellet to hDFs resulted in vesicle internalization as observed by confocal microscopy (FIGS. 23D and 23E). For quantification of dose dependent vesicle internalization, images were obtained 6 (FIGS. 23F, 23G and 23H), 12 (FIGS. 23I and 23J), and 24 h (FIGS. 23K and 23L) after the addition of hCSp-EMVs. Higher concentrations of added particles (20 to 40×10⁹) resulted in significantly higher numbers of vesicle-laden cells, with >90% of cells positive as early as 6 h (FIGS. 23G, 23I, and 23K). Individual cells accumulate particles more rapidly at higher concentrations (FIGS. 23H, 23J, and 23L). The fluorescence per cell reached a plateau with all groups equal in intensity after 24 h of incubation (FIG. 23L) despite persistent differences in percent uptake at steady state (FIG. 23K). Minimal background due to free-dye internalization was observed in the cells incubated with serum containing the lipophilic dye only. Thus, at low vesicle concentrations, cells either take up vesicles or they do not, with comparable capacities among transduced cells. This finding provides indirect evidence against a stochastic process such as membrane fusion, but is consistent with more active mechanisms of EMV uptake (endocytosis or receptor-mediated uptake).

### Example 34

### Validation of In Vitro Biological Activity

OF Experiments performed in vitro to assay the bioactivity of hCSp-EMVs on hDFs revealed dose dependent suppression of phosphorylated small mothers against decapentaplegic homolog (smad)2/3, EMVs. smad4, and snai1, a zinc finger transcription factor and master regulator of epithelial-mesenchymal transition (FIGS. 24A through 24C). These antifibrotic signaling changes mirror those described for CSp-conditioned media. To look for potential conversion of fibroblast phenotype, the Inventors evaluated the expression of fibroblast-specific protein 1 (FSP1), discoidin domain receptor 2 (DDR2), CD105, and CD90, after 24 h of hCSp-EMV incubation. Representative flow cytometry plots (FIG. 24D) and pooled data (FIG. 24E) reveal significant attenuation of both FSP1 and DDR2, but no effects on CD105 or CD90 expression after single exposures to hCSp-EMVs. Immunohistochemistry confirmed the reduced expression of FSP1, but also showed enhanced expression of smooth muscle actin (SMA) (FIGS. 24F and 24G). The secretome of hDFs also changed after exposure to hCSp-EMVs: primed hDFs secreted much higher levels of stromal-cell-derived factor 1 (SDF-1) and vascular endothelial growth factor (VEGF) than unprimed hDFs (FIGS. 24H and 24I). Similar changes were seen in hDFs treated with CSp-exosomes isolated by ultracentrifugation, a complementary isolation method to the default precipitation approach. Ultracentrifugation enriches EMVs and particularly exosomes while excluding protein complexes and other debris. The congruence of the findings with the 2 isolation methods supports the conjecture that EMVs are primarily responsible for the biological effects investigated here.

### Example 35

### Cardioprotective and Angiogenic Effects of hCSp-EMV Primed DFs

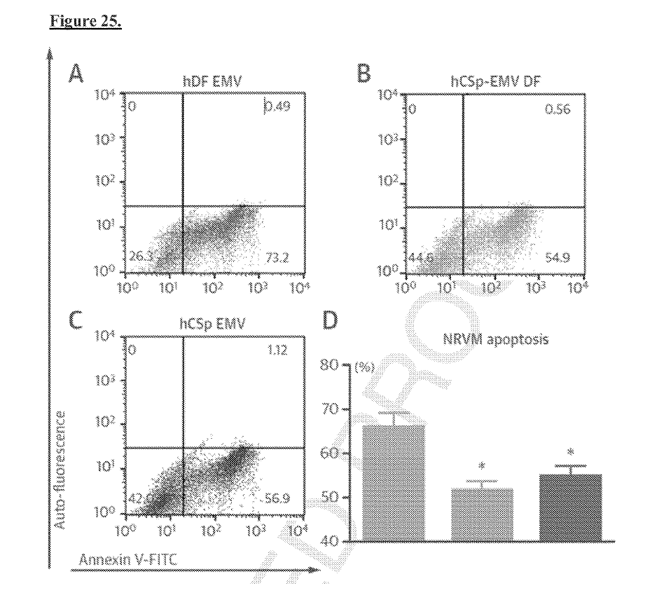

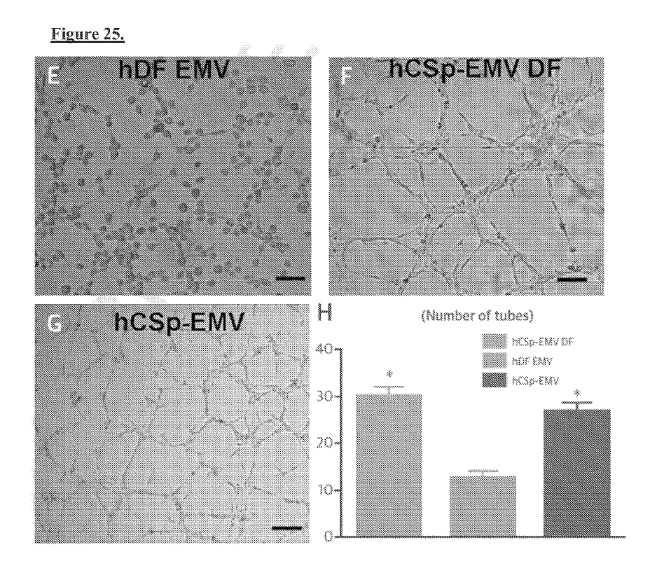

Conditioned media from hCSp-EMV primed hDFs and hCSp-EMVs per se reduced cardiomyocyte apoptosis after oxidative stress, unlike hDF-EMVs (representative fluorescence activated cell sorting [FACS] plots in FIGS. 25A through 25C and pooled data in FIG. 25D). Additionally, in an in vitro matrigel angiogenesis assay, conditioned media from hCSp-EMV_{┐} primed DFs, exerted an angiogenic effect, which was as strong as that of hCSp-EMV alone; both treatments induced significantly more tube formation than hDF-EMVs (representative microscope images in FIGS. 25E through 25G and pooled data in FIG. 25H). Thus, hCSp-EMV priming confers on hDFs the ability to stimulate angiogenesis and to protect cardiomyocytes against stress-induced apoptosis. Enhanced angiogenesis was also observed using hCSp-exosomes isolated by ultracentrifugation, once again indicating that a preparation enriched in exosomes can recapitulate the beneficial effects seen with EMVs isolated by precipitation.

### Example 36

### Distinctive miRNA Profiles of hCSp-EMV Primed DFs

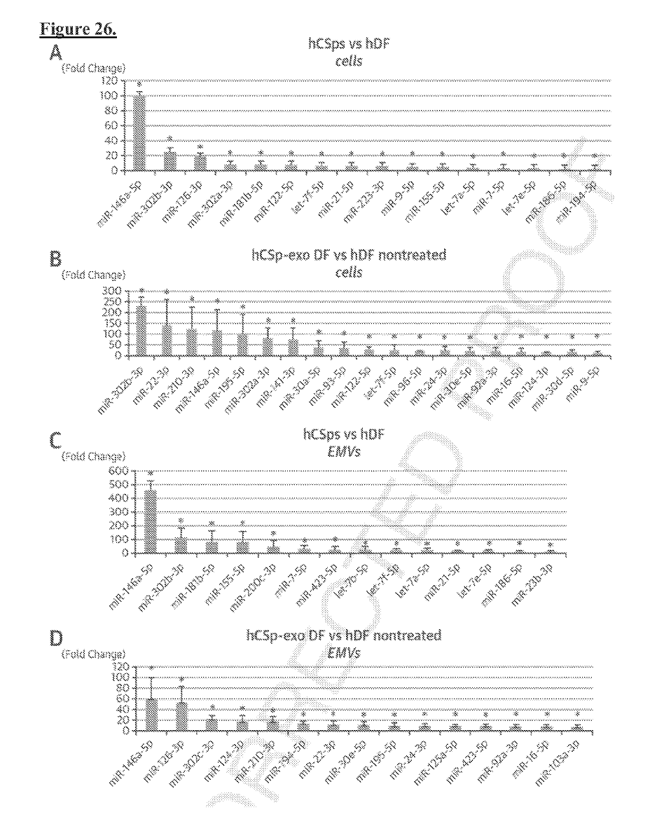

The Inventors previously reported that hCDC derived EMVs, identified as exosomes, express a unique miRNA payload that at least partially accounts for the in vivo regenerative capacity of CDCs. Indeed, this and other reports have led to the conjecture that vesicles affect gene expression of recipient cells by miRNA transfer. To see if this mechanism might be operating here, the Inventors first investigated the global miRNA content of hCSps and compared it to that of hDFs. A number of miRNAs were enriched in hCSps relative to hDFs: FIG. 26A highlights those that are most abundantly overexpressed in hCSps. The Inventors then compared the miRNA profiles of CSp-EMV-primed and unprimed hDFs. FIG. 26B reveals that hCSp-EMV-primed hDFs express very different miRNAs than unprimed hDFs (FIG. 26B). The pattern only partially resembles that of the cells of origin (hCSps; compare to FIG. 26A) or of the vesicles secreted by hCSps (FIG. 26C), hinting that simple passive transfer of vesicular miRNAs cannot fully account for the distinctive miRNA profile of hCSp-EMV-primed hDFs.

Finally, the Inventors compared the miRNA profiles of vesicles secreted by primed and unprimed hDFs by collecting media produced 24 h after priming by hCSp-EMVs or vehicle. The miRNA profiles of primed and unprimed hDFs differed enormously (FIG. 26D). The miRNAs secreted by hCSp-EMV-primed hDFs include several that are enriched in hCSp-EMVs themselves (notably miRNA-146a, which was highlighted by Ibrahim et al. and is elevated in all therapeutically active groups here), but the patterns are otherwise quite individual. Thus, priming with hCSp-EMVs leads to fundamental changes in hDF miRNA expression profiles and hDF secreted vesicles. The distinctive miRNA profiles in hCSp-EMV-primed hDFs and their membrane vesicles argue against the possibility that the changes merely reflect accumulation and subsequent “regurgitation” of miRNAs transferred in hCSp-EMVs.

### Example 37

### rCSp-EMV-Primed Fibroblasts Reverse Remodeling

The Inventors have presented evidence that hCSp-EMV-primed hDFs secrete SDF-1 and VEGF, exert anti-apoptotic and angiogenic effects in vitro, and express distinctive miRNAs. The Inventors therefore questioned whether rCSp-EMVs themselves, as well as rCSp-EMV-primed DFs, might confer therapeutic benefits in vivo in a rat model of chronic MI. One month after permanent left anterior descending ligation, animals underwent intramyocardial injection.

To assess particle biodistribution qualitatively, animals (n=3) injected with dye-labeled rCSp-EMVs were euthanized 1 h post-injection and selected organs were imaged. Approximately 20% of the injected rCSp-EMVs were found in the heart; the lungs also exhibited obvious uptake, with less in other organs. This percentage of retention in the heart at 1 h compares favorably with that seen with intramyocardially injected cells. Minimal intensity was detected by the free dye control injections only.

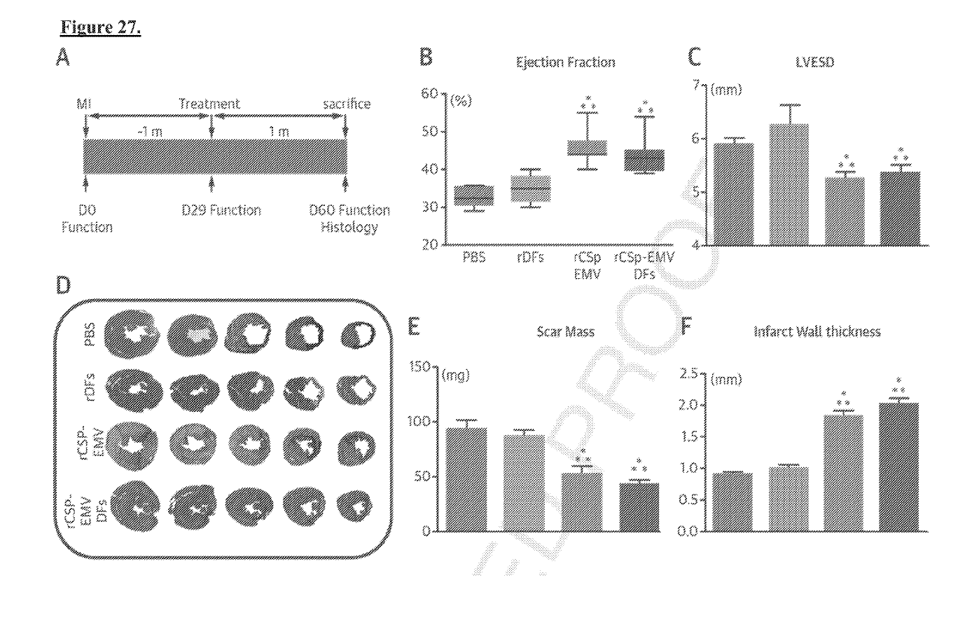

For long-term physiological experiments, animals were randomly allocated to 1 of the following 4 groups: vehicle (PBS), unprimed rDFs, rCSp-EMVs, or rCSp-EMV-primed rDFs with treatment at 1 month post-MI (FIG. 27A). One month later, echocardiography revealed improved ejection fraction in the rCSp-EMVs and the rCSp-EMV_{┐} primed rDFs groups compared to either vehicle or unprimed rDFs (FIG. 27B). This finding primarily reflected differences in left ventricular end-systolic diameter (FIG. 27C). Histological analysis (of samples exemplified in FIG. 27D) showed significant reductions in scar mass (FIG. 5E) and enhanced infarct wall thickness (FIG. 27F) in the rCSp-EMVs and rCSp-EMV-primed rDF groups. The structural and functional improvements seen with rCSp-EMVs and rCSp-EMV-primed rDFs were comparable to those reported with rCSp injection in this model.

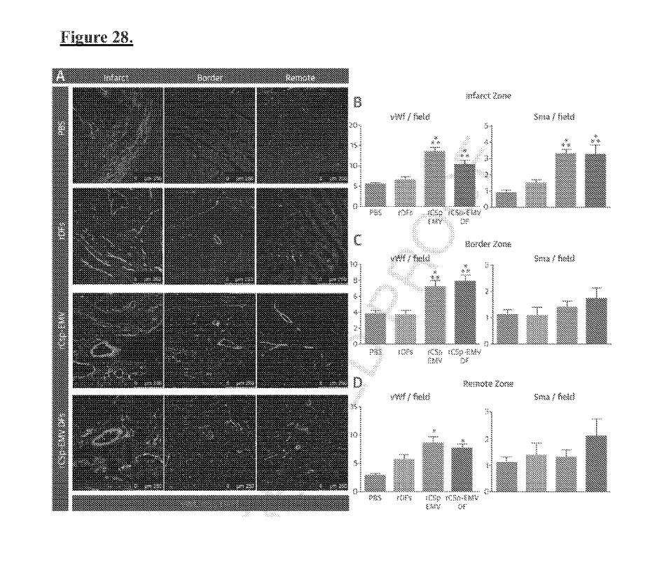

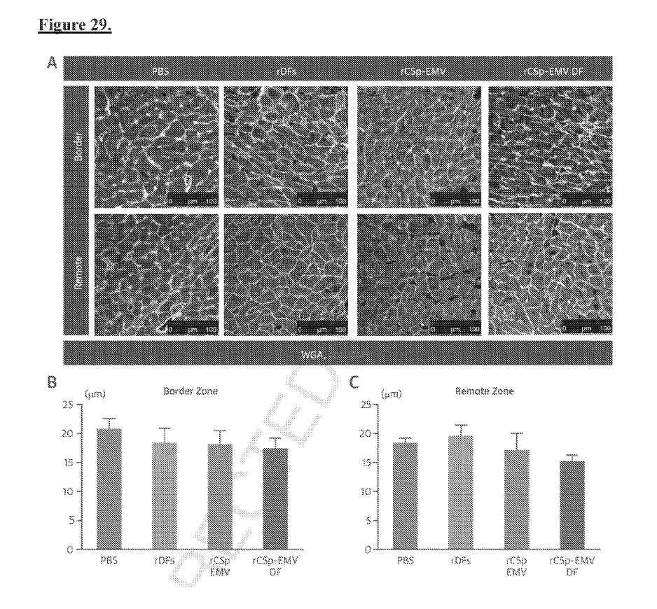

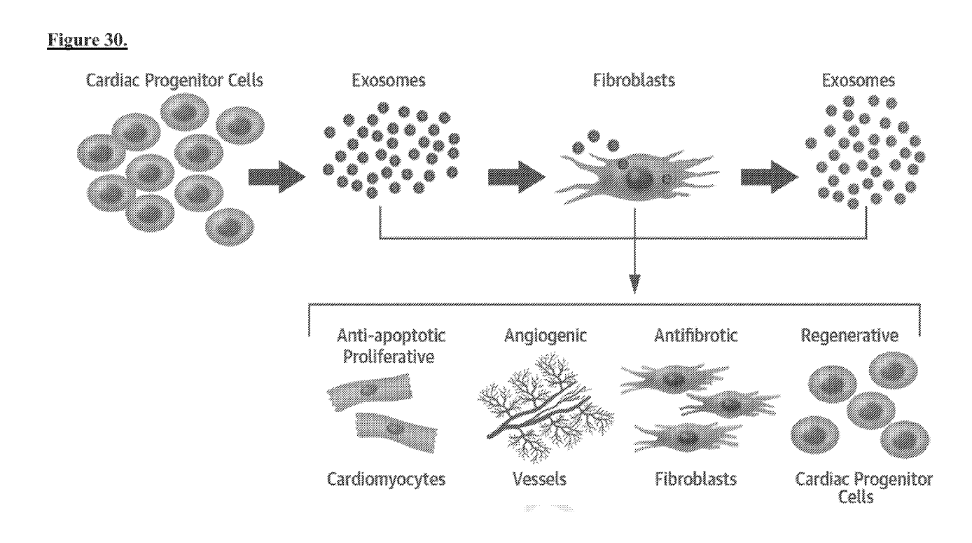

Finally, to test the in vivo angiogenic capacity of CSp-EMVs and CSp-EMV-primed cells, the Inventors quantified capillaries (bounded by von Willebrand factor positive cells) and microvessels (bounded by SMA positive cells; FIG. 28A). Analysis of serial images from the apex to the base revealed greater capillary density in the rCSp-EMVs and rCSp-EMV-primed groups compared to both controls in all 3 zones evaluated (infarct, border, and remote) (FIGS. 28B through 28D, left panel). Microvessel density was likewise increased, but only in the infarct zone (FIGS. 28B through 28D, right panel). Another mechanism underlying CSp regenerative efficacy is cardiomyocyte proliferation. Bromodeoxyuridine incorporation revealed enhanced deoxyribonucleic acid synthesis after exposure to rCSp-EMVs and rCSp-EMV-rDFs compared to PBS and unprimed rDFs, validating previous reports. Interestingly, this effect tended to be more prominent after rCSp-EMV-only injections compared to rCSp-EMVrDFs. Finally, no changes in cardiomyocyte diameter were observed (FIGS. 29A through 29C).

### Example 38

### Systemic Administration with Splenic Macrophage Polarization

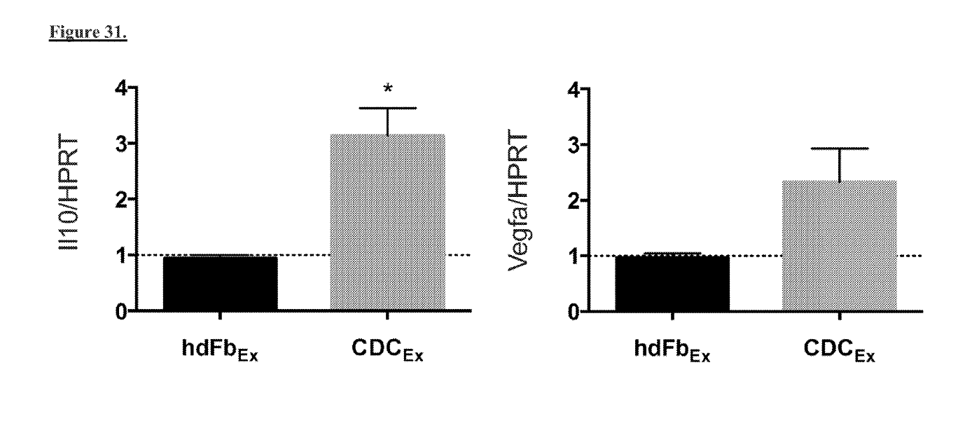

Here the Inventors show data in mice that splenic mononuclear cells (which include macrophages) are uniquely polarized following treatment with human CDC exosomes (CDCexo). To do so, the Inventors pretreated mice with an intraperitoneal injection of lipopolysaccharide (LPS), an acute inflammatory stimulus, then infused CDCexo, or human dermal fibroblasts (hdFbexo) into the carotid artery. Eighteen hours later, mice were sacrificed and spleens collected. Spleens were digested to obtain a mixed cellular suspension. Mononuclear cells were isolated by density gradient centrifugation and plating onto cell culture dishes. Following attachment, cells were collected for RNA isolation and cDNA synthesis. Quantitative RT-PCR was then performed to assess the gene expression levels of Il10 and Vegfa, both of which were found upregulated in CDCexo-treated, but not Fbexo-treated, animals (FIG. 31).

### Example 39

### Discussion

The Inventors have observed remarkable effects of EMVs from hCSps on fibroblasts. DFs are venerable controls for cardiac cell therapy; their injection neither improves nor aggravates adverse remodeling after MI. DF produced exosomes are likewise inert. In contrast, cardiospheres and their progeny trigger functional recovery and structural improvements in various ischemic and nonischemic models of heart failure. This beneficial effect was recently attributed to secreted exosomes. Although interaction of EMVs with endothelial cells and cardiomyocytes has been reported, the Inventors' data support a strong, previously unappreciated bioactivity of CSp-EMVs on fibroblasts and other cardiac cell types (Central Illustration). More specifically, the Inventors report that, in the restricted environment of in vitro priming with hCSp-EMVs, hDFs exert a dose-dependent downregulation of the transforming growth factor-beta cascade and increased secretion of SDF-1 and VEGF. Remarkably, these primed fibroblasts promote angiogenesis and inhibit cardiomyocyte apoptosis in vitro, whereas in vivo they can attenuate remodeling and improve function to levels equivalent to those reported with rCSps.

Vesicular transfer of miRNAs mediates cell-cell communication in different biological systems. However, the miRNA cargo of EMVs does not necessarily reflect passive loading with RNAs in the parent cell; selective enrichment mechanisms appear to be at play. This selective miRNA payload may be a crucial determinant of bioactivity on the recipient population. Indeed, the Inventors found a distinct miRNA signature in primed versus unprimed hDFs that does not reflect passive release of internalized hCSp-EMVs.

Therefore, internalization of hCSp-EMVs leads to downstream, biologically significant changes in miRNA vesicular cargo released by the recipient hDFs. Additionally, because fibroblast-derived EMVs enriched in miRNAs do not improve recovery in vivo, the cargo transition described here may provide promising clues to pathways involved in reverse remodeling.

Many studies report the innate plasticity of fibroblasts that allows them to acquire a cardiomyocyte or endothelial phenotype after exposure to either transcription factors or small molecules. These direct reprogramming approaches may constitute a promising therapeutic strategy. The Inventors' data indicate that single-dose priming with CSp-EMVs converted DFs to a less fibrotic phenotype with functional properties equal to those of CSp-EMVs. Interestingly, the transforming growth factor-beta pathway, which provides key signals in cellular conversion, was significantly downregulated (FIGS. 24A through 24C). The Inventors do not yet know if EMVs or an EMV subgroup (e.g., exosomes) suffice to durably reprogram DFs to a fully-distinct cell type, but the Inventors' data do indicate that inert fibroblasts can be functionally converted both in vitro and in vivo for a sufficient duration to shape therapeutic activity.

Crosstalk between endothelial cell-derived vesicles containing miRNAs and the surrounding myocardium has been reported. Here the Inventors showed that, beyond strictly paired cell-cell communication, divergent transportation of biologically significant signals takes place between hCSp-EMV-primed DFs and recipient human umbilical vein endothelial cells or cardiomyocytes. These findings in culture may help to rationalize the efficacy of a relatively low number of cells injected in vivo: transplanted cells secrete EMVs, which interact with the surrounding tissue and convert it into a more salutary milieu, in a positive feedback loop. Xenogeneic transplantation of CSps post-MI elicits detrimental immunological sequalae, although allogeneic CSps are effective and immunologically innocuous. Here the Inventors used allogeneic rCSp-EMVs to evaluate potency in vivo and allogeneic hCSp-EMVs for in vitro experiments. Evaluation of the immunological sequalae of allogeneic and xenogeneic EMVs is beyond the scope of the present study, but it seems likely that CSp-EMVs may be even less immunogenic than CSps, as they express far fewer surface antigens and, unlike cells, cannot react dynamically to immunological cues.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are sources of cardiosphere derived cells, the use of alternative sources such as cells derived directly from heart biopsies (explant-derived cells), or from self-assembling clusters of heart-derived cells (cardiospheres), exosomes produced by such cells, method of isolating, characterizing or altering exosomes produced by such cells, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1-33. (canceled)

34. A method of modulating inflammation, comprising:
administering a composition comprising a plurality of exosomes to a subject afflicted with an inflammatory related disease or condition, wherein administration of the composition modulates inflammation in the subject by polarizing a population of macrophages in the subject.

35. The method of claim 34, wherein the inflammatory related disease or condition is acute or chronic.

36. The method of claim 34, wherein the inflammatory related disease or condition is a heart related disease or condition.

37. The method of claim 36, wherein the heart related disease or condition is myocardial infarct.

38. The method of claim 36, wherein the heart related disease or condition is atherosclerosis or heart failure.

39. The method of claim 34, wherein polarizing a population of macrophages comprises appearance of one or more of: M_{CDC} macrophage phenotype, decreased M1 macrophage phenotype and increased M2 macrophage phenotype.

40. The method of claim 39, wherein the M_{CDC} macrophage phenotype comprises expression of one or more of: interleukin-10 (Il10) and interleukin-4ra (Il4ra), M1 macrophage phenotype comprises expression of one or more of: nitric oxidate synthase (Nos2), tumor necrosis factor (Tnf), interleukin-1 (Il1), and interleukin6 (Il6), and M2 macrophage phenotype comprises expression of one or more of: arginase 1 (Arg1), interleukin-10 (Il10), and peroxisome proliferator-activated receptor gamma (Pparg).

41. The method of claim 39, wherein decreased M1 macrophage phenotype or increased M2 macrophage phenotype comprises an increase in Arg1/Nos2 ratio in a population of macrophages.

42. The method of claim 39, wherein decreased M1 macrophage phenotype or increased M2 macrophage phenotype comprises a decrease in Ly6C expression in a population of macrophages.

43. The method of claim 39, wherein the macrophages are from cardiac tissue, peritoneum, spleen or bone marrow.

44. The method of claim 34 wherein administering a composition comprises 1×10⁸ or more exosomes in a single dose.

45. The method of claim 44, wherein a single dose is administered multiple times to the subject.

46. The method of claim 34, wherein administering a composition consists of one or more of: intra-arterial infusion, intravenous infusion, percutaneous injection, and injection directly into heart tissue.

47. A method of conferring cardioprotection, comprising:
administering a composition comprising a plurality of exosomes to a subject afflicted with myocardial infarct (MI), ischemia/reperfusion (IR), or both, wherein administration of the composition confers cardioprotection by polarizing a population of macrophages in the subject.

48. The method of claim 47, wherein the macrophages are from cardiac tissue, peritoneum, spleen or bone marrow.

49. The method of claim 47, wherein administering a composition comprises 1×10⁸ or more exosomes in a single dose.

50. The method of claim 49, wherein a single dose is administered multiple times to the subject.

51. The method of claim 47, wherein administering a composition consists of one or more of: intra-arterial infusion, intravenous infusion, percutaneous injection, and injection directly into heart tissue.

52. The method of claim 47, wherein administering a composition comprising a plurality of exosomes to the subject is adjunctive to standard therapy.

53. The method of claim 47, wherein administering a composition is less than 1 hour after reperfusion.

54. The method of claim 47, wherein conferring cardioprotection reduces infarct size.

55. An in vitro method of altering a cell, comprising:
providing a plurality of exosomes; and
adding to a starting cell type, the plurality of exosomes, wherein adhesion between exosomes in the plurality of exosomes and the starting cell type is capable of altering one or more properties of the starting cell type, and generating a converted cell type.

56. The method of claim 55, wherein the plurality of exosomes are derived from stem cells, progenitors, or precursor cells.

57. The method of claim 56, wherein the stem cells, progenitors, or precursor cells comprise cardiosphere-derived cells (CDCs).

58. The method of claim 56, wherein the stem cells, progenitors, or precursor cells comprise endothelial precursor cells (EPCs) or mesenchymal stem cells (MSCs).

59. The method of claim 55, wherein the starting cell type is a fibroblast.
